(19) United States
(12) Patent Application Publication
(10) Pub. No.: US 2022/0145291 A1
(43) Pub. Date: May 12, 2022

(54) Extracellular Vesicle Functionalization Using Oligonucleotide Tethers

(71) Applicant: Carnegie Mellon University, Pittsburgh, PA (US)

(72) Inventors: Subha Ranjan Das, Pittsburgh, PA (US); Phil G. Campbell, Pittsburgh, PA (US); Krzysztof Matyjaszewski, Pittsburgh, PA (US); Sushil Lathwal, Pittsburgh, PA (US); Saigopalakrishna Saileelaprasad Yernei, Pittsburgh, PA (US)

(21) Appl. No.: 17/430,837

(22) PCT Filed: Feb. 14, 2020
(86) PCT No.: PCT/US20/18303

§ 371 Date: Aug. 13, 2021

Related U.S. Application Data
(60) Provisional application No. 62/918,817, filed on Feb. 14, 2019.

(51) Int. Cl. C12N 15/11; A61K 47/54; A61K 47/60; A61K 47/69

(52) U.S. Cl. CPC C12N 15/111; A61K 47/549; C08L 33/26; A61K 47/6901; A61K 47/6903; A61K 47/60

(57) ABSTRACT

Provided herein are tethered extracellular vesicles and methods of making tethered extracellular vesicles.

### CROSS REFERENCE TO RELATED APPLICATIONS

This application is the United States national phase of International Application No. PCT/US2020/018303 filed Feb. 14, 2020, and claims the benefit of U.S. Provisional Patent Application No. 62/918,817, filed Feb. 14, 2019, the disclosures of which are hereby incorporated by reference in their entirety.

### REFERENCE TO A SEQUENCE LISTING SUBMITTED VIA EFS-WEB

The content of the ASCII text file of the sequence listing named 2103522_ST25.txt which is 3,259 bytes in size was created on Aug. 13, 2021 and electronically submitted via EFS-Web herewith the application is incorporated herein by reference in its entirety.

A rapid and versatile method for functionalization of extracellular vesicles using oligonucleotide tethers allowing incorporation of targeting ligands, or any indeed specific protein or functional entity of interest, has been developed. The disclosed exemplary DNA tether-based exosome functionalization strategy was also employed to engineer exosomes with linked functional polymers thereby preparing exosome-polymer hybrids (EPHs). EPHs showed improved stability and provided tunable surface properties, compared to their native vesicle counterparts. Since exosomes have native tissue-targeting properties it was essential that the procedures developed were also able to conserve their intrinsic biological properties post derivatization. Cellular studies of these functionalized exosomes highlighted their robustness and confirmed positive results for a range of applications. This work has laid the groundwork for a formation of a novel class of biohybrids with potential to overcome the limitations of current drug delivery systems, including delivery of drugs across the blood brain barrier.

Extracellular vesicles (EVs), including exomeres, exosomes, micro-vesicles or apoptotic bodies can be functionalized in the manner exemplified herein by the functionalization of exosomes (30-150 nm in size) and micro-vesicles (200 nm-1 μm). EVs are bilayer lipid membrane-bound vesicles containing proteins and nucleic acids such as microRNA (miRNAs), mRNA, and DNA. EVs are released from many, if not all, cell types in the body. They play a key role in intercellular communication in autocrine, paracrine and telecrine pathways. The ability of EVs to selectively transport proteins, lipids and nucleic acids to cells has created interest in the field of drug delivery, where efficient and targeted delivery of bio-active molecules is desired, and otherwise difficult to achieve with synthetic systems such as liposomes. Multiple studies have shown that the use of EVs for therapeutic purposes is feasible, and EVs have even already been applied in phase 1 clinical trials (Gyorgy et al., “Therapeutic applications of extracellular vesicles: clinical promise and open questions”, Annu Rev Pharmacol Toxicol, 2015, 55: 439-64; Lener et al., “Applying extracellular vesicles based therapeutics in clinical trials—an ISEV position paper”, J Extracell Vesicles, 2015, 4, 30087).

Exosomes are not only some of the smallest EVs but are of particular interest due to their unique characteristics, such as their ability to cross the blood brain barrier. Moreover, the biogenesis of exosomes is unique: they originate from the endocytic compartment of cells and their molecular content reflects, at least in part, that of the parental cell. However, native exosomes may also possess undesirable properties that could limit their application as drug delivery systems. For example, their natural bioactive payloads may counteract the desired therapeutic effects, and a lack of targeting specificity may result in uptake by non-targeted, healthy cells. The presence of functional extracellular entities disclosed herein can overcome such issues.

Multiple reports have shown that exosomes can be engineered to include specific cargo within the membrane, or express targeting ligands, to improve their drug delivery potential. However, previously described targeting strategies have been mainly based on the fusion of targeting ligands with exosome membrane proteins, such as Lamp2b. Such strategies have several drawbacks; e.g., the function of exosome membrane proteins, such as fusion with cellular membranes or immune regulation, may be compromised upon fusion with targeting ligands. For example, some Lamp2b-fused targeting ligands have been describes as undergoing premature degradation instead of functional display of exosomes (Hung et al. “Stabilization of exosome-targeting peptides via engineered glycosylation”, J Biol Chem, 2015, 290: 8166-72). Moreover, scalability can be an issue when dealing with engineering exosome producing cells or bioengineering through cells.

To avoid such issues, multiple groups have explored strategies to functionalize exosome surfaces post cellular secretion, circumventing the need to modify EV producing cells (Kooijmans et al., J Control Release, 2016; Smyth et al. “Surface functionalization of exosomes using click chemistry”, Bioconjug Chem, 2014, 25: 1777-84; O'Loughlin et al. “Functional delivery of lipid-conjugated siRNA by extracellular vesicles” Molecular Therapy, 2017, 25(7): 1580-7). For example, Smyth and co-workers grafted alkyne moieties onto isolated exosomes to link these vesicles to fluorescent probes using click chemistry. On the other hand, O'Loughlin and co-workers used hydrophobically modified siRNA for exosome loading.

Development of hybrid systems utilizing membrane fusion of cells with synthetic liposomes have also been reported. Unfortunately, such modifications, including the need for dual lipid based anchoring strands, may also compromise the functionality of crucial exosome components for exosome-cell interactions and cargo delivery. There was one report of using a cholesterol modified DNA (chol-DNA) to anchor vesicles onto a solid surface (Pfeiffer, I, et al. Bivalent Cholesterol-Based Coupling of Oligonucletides to Lipid Membrane Assemblies J. Am. Chem. Soc. 2004, 126, 33, 10224-10225), and another the utilized chol-siRNA directly as a drug (Jeong, J H, et al., siRNA Conjugate Delivery Systems Bioconjugate Chem, 2009, 20:5-14), neither indicating any direct interaction with the lipid bilayer of vesicles.

Despite encouraging results, therapeutic potential of exosomes is largely restricted by their short stability, limited tools for modification, and by the rapid clearance of exogenously administered exosomes from blood post-injection.

### SUMMARY OF THE INVENTION

According to one aspect of the invention, provided herein is a tethered extracellular vesicle comprising: an extracellular vesicle sourced from any of the domains of life; a hydrophobically-modified first oligonucleotide anchored to the extracellular vesicle; and a second oligonucleotide hybridized to the first oligonucleotide linked to a member of a binding pair, a therapeutic agent, a surface, or a polymer.

In another aspect, provided herein is a tethered extracellular vesicle comprising: an extracellular vesicle; and a hydrophobically-modified oligonucleotide anchored to the extracellular vesicle and linked to a polymer.

A hydrogel comprising two or more of the tethered extracellular vesicles described in the previous paragraphs also is provided, wherein the polymer of the two or more tethered extracellular vesicles is cross-linked with a cross-linker. The tethered extracellular vesicles and/or hydrogel may be associated with a therapeutic agent.

In another aspect, a method of making a tethered extracellular vesicle is provided, comprising: anchoring a hydrophobically-modified oligonucleotide to an extracellular vesicle; hybridizing to the hydrophobically-modified oligonucleotide a second oligonucleotide complementary to the hydrophobically-modified oligonucleotide and linked to a member of a binding pair, a therapeutic agent, a surface, a polymer initiator group, or a polymer.

In yet another aspect, a method of making a tethered extracellular vesicle is provided, comprising: anchoring a hydrophobically-modified oligonucleotide comprising a polymer initiator group to the extracellular vesicle; and polymerizing a polymer in a polymerization reaction from the polymer initiator group.

Non-limiting aspects or embodiments of the present invention will now be described in the following numbered clauses:

Clause 1. A tethered extracellular vesicle comprising:
an extracellular vesicle sourced from any of the domains of life;
a hydrophobically-modified first oligonucleotide anchored to the extracellular vesicle; and
a second oligonucleotide hybridized to the first oligonucleotide linked to a member of a binding pair, a therapeutic agent, a surface, or a polymer.
   Clause 2. The tethered extracellular vesicle of clause 1, wherein the second oligonucleotide is linked to a polymer.
   Clause 3. The tethered extracellular vesicle of clause 2, wherein the polymer is a polyacrylate, a polymethacrylate, a polyacrylamide, a polymethacrylamide, a polypeptide, a polystyrene, a polyethylene oxide, a poly(organo)phosphazene, a poly-L-lysine, a polyethyleneimine, a poly-d,l-lactide-co-glycolide, or a poly(alkylcyanoacrylate)
   Clause 4. The tethered extracellular vesicle of clause 2, wherein the polymer has a saturated carbon backbone and/or is prepared from one or more ethylenically unsaturated monomers.
   Clause 5. The tethered extracellular vesicle of any one of clauses 2-4, wherein the polymer has a PDI of less than 2.0, less than 1.75, less than 1.5, or less than 1.2.
   Clause 6. The tethered extracellular vesicle any one of clauses 2-5, wherein the polymer is an acrylic polymer.
   Clause 7. The tethered extracellular vesicle of clause 4, wherein the acrylic polymer comprises pendant poly(ethylene oxide) groups having the structure —(O—CH₂—CH₂—)ₙ, where n is 100 or less, 20 or less or 10 or less; zwitterionic groups; or methylsulfinyl terminated alkyl groups.
   Clause 8. The tethered extracellular vesicle of clause 5, wherein the acrylic polymer comprises pendant poly(ethylene oxide) groups.
   Clause 9. The tethered extracellular vesicle of clause 6, wherein the pendant poly(ethylene oxide) groups have an Mₙ of 200 or less.
   Clause 10. The tethered extracellular vesicle of any one of clauses 2-6, wherein the polymer is cross-linked, forming a hydrogel comprising the EV
   Clause 11. The tethered extracellular vesicle of clause 1, wherein the second oligonucleotide is linked to a biologically active agent, such as a therapeutic agent.
   Clause 12. The tethered extracellular vesicle of clause 1, wherein the second oligonucleotide is linked to a binding reagent, such as an antibody, an antibody fragment, or an aptamer.
   Clause 13. The tethered extracellular vesicle of clause 12, wherein the binding reagent is complexed with a biologically active agent, such as a therapeutic agent.
   Clause 14. The tethered extracellular vesicle of clause 1, wherein the extracellular vesicle comprises a therapeutic agent, such as a therapeutic loaded inside the lumen of the extracellular vesicle or on the membrane surface.
   Clause 15. The tethered extracellular vesicle of any one of clauses 1-14, wherein the hydrophobically-modified oligonucleotide is an oligonucleotide linked to a sterol, such as cholesterol, GM1, a lipid, a vitamin, a small molecule, or a peptide, or a combination thereof.
   Clause 16. The tethered extracellular vesicle of any one of clauses 1-15, wherein the extracellular vesicles are exosomes.
   Clause 17. A composition comprising the tethered extracellular vesicle of any one of clauses 1-16, and a pharmaceutically-acceptable excipient.
   Clause 18. A hydrogel comprising two or more of the tethered extracellular vesicles any of clauses 2-13, wherein the polymer of the two or more tethered extracellular vesicles is cross-linked with a cross-linker.
   Clause 19. The hydrogel of clause 18, wherein the polymer comprises a saturated carbon backbone and is functionalized with poly(ethylene oxide)-containing groups, and the cross-linker comprises poly(ethylene oxide).
   Clause 20. The hydrogel of clause 18 or 19, comprising a biologically active agent, such as a therapeutic agent, such as a therapeutic loaded inside the lumen of the extracellular vesicle or on the membrane surface.
   Clause 21. The hydrogel of clause 20, wherein the biologically active agent is tethered to the extracellular vesicle by attachment to, or complexing with the hydrophobically-modified oligonucleotide.
   Clause 22. A tethered extracellular vesicle comprising:
an extracellular vesicle; and
a hydrophobically-modified oligonucleotide anchored to the extracellular vesicle and linked to a polymer.
   Clause 23. The tethered extracellular vesicle of clause 22, wherein the polymer is a polyacrylate, a polymethacrylate, a polyacrylamide, a polymethacrylamide, a polypeptide, a polystyrene, a polyethylene oxide, a poly(organo)phosphazene, a poly-L-lysine, a polyethyleneimine, a poly-d,l-lactide-co-glycolide, or a poly(alkylcyanoacrylate).
   Clause 24. The tethered extracellular vesicle of clause 23, wherein the polymer has a saturated carbon backbone and/or is prepared from one or more ethylenically unsaturated monomers.
   Clause 25. The tethered extracellular vesicle of clause 22 or 23, wherein the polymer has a dispersity (D) of less than 2.0, less than 1.75, less than 1.5, or less than 1.2.
   Clause 26. The tethered extracellular vesicle of any one of clauses 22-25, wherein the polymer is an acrylic polymer.
   Clause 27. The tethered extracellular vesicle of any one of clauses 22-26, wherein the polymer comprises a pendant zwitterionic moiety, such as a carboxybetaine moiety, and/or a pendant methylsulfinylalkyl moiety.
   Clause 28. The tethered extracellular vesicle of any one of clauses 22-26, wherein the acrylic comprises pendant poly(ethylene oxide) groups having the structure —(O—CH₂—CH₂—)ₙ, where n is 100 or less, 20 or less or 10 or less.
   Clause 29. The tethered extracellular vesicle of clause 28, wherein the pendant poly(ethylene oxide) groups have an Mₙ of 200 or less.
   Clause 30. The tethered extracellular vesicle of any one of clauses 22-29, wherein the hydrophobically-modified oligonucleotide is an oligonucleotide linked to a sterol, such as cholesterol, GM1, a lipid, a vitamin, a small molecule, or a peptide, or a combination thereof.
   Clause 31. A composition comprising the tethered extracellular vesicle of any one of clauses 22-30, and a pharmaceutically-acceptable excipient.
   Clause 32. A hydrogel comprising two or more of the tethered extracellular vesicles any of clauses 22-30, wherein the polymer of the two or more tethered extracellular vesicles is cross-linked with a cross-linker.
   Clause 33. The hydrogel of clause 32, wherein the polymer comprises a saturated carbon backbone and is functionalized with poly(ethylene oxide)-containing groups, and the cross-linker comprises poly(ethylene oxide) groups, the poly(ethylene oxide) groups having the structure —(O—CH₂—CH₂—)ₙ, where n optionally is 100 or less, 20 or less or 10 or less.
   Clause 34. The hydrogel of clause 32 or 33, comprising a biologically active agent, such as a therapeutic agent, such as a therapeutic loaded inside the lumen of the extracellular vesicle or on the membrane surface.
   Clause 35. The hydrogel of clause 34, wherein the biologically active agent is tethered to the extracellular vesicle by attachment to, or complexing with the hydrophobically-modified oligonucleotide.
   Clause 36. A method of making a tethered extracellular vesicle, comprising:
anchoring a hydrophobically-modified oligonucleotide to an extracellular vesicle;
hybridizing to the hydrophobically-modified oligonucleotide a second oligonucleotide complementary to the hydrophobically-modified oligonucleotide and linked to a member of a binding pair, a therapeutic agent, a surface, a polymer initiator group, or a polymer.
   Clause 37. The method of clause 36, wherein the hydrophobically-modified oligonucleotide is an oligonucleotide linked to a sterol, such as cholesterol, GM1, a lipid, a vitamin, a small molecule, or a peptide, or a combination thereof.
   Clause 38. The method of clause 36 or 37, wherein the second oligonucleotide is linked to a polymer.
   Clause 39. The method of clause 38, wherein the polymer is a polyacrylate, a polymethacrylate, a polyacrylamide, a polymethacrylamide, a polypeptide, a polystyrene, a polyethylene oxide, a poly(organo)phosphazene, a poly-L-lysine, a polyethyleneimine, a poly-d,l-lactide-co-glycolide, or a poly(alkylcyanoacrylate).
   Clause 40. The method of clause 38, wherein the polymer is prepared from one or more ethylenically unsaturated monomers.
   Clause 41. The method of any one of clauses 38-40, wherein the polymer has a PDI of less than 2.0, less than 1.75, less than 1.5, or less than 1.2.
   Clause 42. The method of any one of clauses 38-41, wherein the polymer is an acrylic polymer.
   Clause 43. The method of any one of clauses 38-42, wherein the polymer comprises pendant poly(ethylene oxide) groups having the structure —(O—CH₂—CH₂—)ₙ, where n is 100 or less, 20 or less or 10 or less; zwitterionic groups; or methylsulfinyl terminated alkyl groups.
   Clause 44. The method of any one of clauses 38-43, wherein the polymer is an acrylic polymer comprising pendant poly(ethylene oxide) groups.
   Clause 45. The method of clauses 44, wherein the pendant poly(ethylene oxide) groups have an Mₙ of 200 or less.
   Clause 46. The method of any one of clauses 38-45, further comprising, after hybridizing the second oligonucleotide to the hydrophobically-modified oligonucleotide, cross-linking the polymer, forming a hydrogel comprising the extracellular vesicles, wherein the polymer optionally comprises a saturated carbon backbone and is functionalized with poly(ethylene oxide)-containing groups, and the cross-linker comprises poly(ethylene oxide) groups, the poly(ethylene oxide) groups having the structure —(O—CH₂—CH₂—)ₙ, where n optionally is 100 or less, 20 or less or 10 or less.
   Clause 47. The method of clause 38, wherein the second oligonucleotide is linked to a polymer initiator, such as an ATRP initiator and further comprising, polymerizing a polymer in a polymerization reaction from the initiator group of the oligonucleotide.
   Clause 48. The method of clause 47, wherein the polymerization reaction is conducted with ethylenically unsaturated monomers.
   Clause 49. The method of clause 47, wherein the polymerization reaction is conducted using controlled radical polymerization.
   Clause 50. The method of clause 49, wherein the polymerization reaction is conducted using atom transfer radical polymerization, such as Activators ReGenerated by Electron Transfer (ARGET) ATRP, Initiators for Continuous Activator Regeneration (ICAR) ATRP, supplemental activator and reducing agent atom transfer radical polymerization (SARA) ATRP, electrochemically-controlled ATRP (e-ATRP), or photoinduced ATRP.
   Clause 51. The method of any one of clauses 47-50, wherein the polymerization reaction is conducted with monomers including poly(ethylene oxide)-substituted acrylate monomers, such as poly(ethylene oxide) groups having the structure —(O—CH₂—CH₂—)ₙ, where n is 100 or less, 20 or less or 10 or less, or poly(ethylene oxide) groups having an Mₙ of 200 or less.
   Clause 52. The method of any one of clauses 47-50, wherein the polymerization reaction is conducted with monomers including zwitterionic-substituted or methylsulfinyl terminated alkyl-substituted acrylate monomers.
   Clause 53. The method of any one of clauses 47-52, further comprising, while the polymer is polymerized or after the polymer is polymerized, cross-linking the polymer, forming a hydrogel comprising the extracellular vesicles, wherein the polymer optionally comprises a saturated carbon backbone and is functionalized with poly(ethylene oxide)-containing groups, and the cross-linker comprises poly(ethylene oxide) groups, the poly(ethylene oxide) groups having the structure —(O—CH₂—CH₂—)ₙ, where n optionally is 100 or less, 20 or less or 10 or less.
   Clause 54. The method of clause 36 or 37, wherein the second oligonucleotide is linked to a biologically active agent, such as a therapeutic agent, such as a therapeutic loaded inside the lumen of the extracellular vesicle or on the membrane surface.
   Clause 55. The method of clause 36 or 37, wherein the second oligonucleotide is linked to a binding reagent, such as an antibody, an antibody fragment, or an aptamer.
   Clause 56. The method of clause 55, further comprising complexing the binding reagent with a biologically active agent, such as a therapeutic agent.
   Clause 57. A method of making a tethered extracellular vesicle, comprising:
anchoring a hydrophobically-modified oligonucleotide comprising a polymer initiator group to the extracellular vesicle; and
polymerizing a polymer in a polymerization reaction from the polymer initiator group.
   Clause 58. The method of clause 57, wherein the polymerization reaction is conducted with ethylenically unsaturated monomers.
   Clause 59. The method of clause 57, wherein the polymer is polymerized using controlled radical polymerization reaction.
   Clause 60. The method of clause 57, wherein the polymer is polymerized using atom transfer radical polymerization, such as Activators ReGenerated by Electron Transfer (ARGET) ATRP, Initiators for Continuous Activator Regeneration (ICAR) ATRP, supplemental activator and reducing agent atom transfer radical polymerization (SARA) ATRP, electrochemically-controlled ATRP (e-ATRP), or photoinduced ATRP.
   Clause 61. The method of any one of clauses 57-60, wherein the polymerization reaction is conducted with monomers including poly(ethylene oxide)-substituted monomers, such as poly(ethylene oxide) groups having the structure —(O—CH₂—CH₂—)ₙ, where n is 100 or less, 20 or less or 10 or less, or poly(ethylene oxide) groups having an Mₙ of 200 or less, wherein the poly(ethylene oxide)-substituted monomers are optionally acrylate monomers.
   Clause 62. The method of any one of clauses 57-61, wherein the polymerization reaction is conducted with monomers including zwitterionic-substituted or methylsulfinyl terminated alkyl-substituted monomers, wherein the zwitterionic-substituted or methylsulfinyl terminated alkyl-substituted monomers are optionally acrylate monomers.
   Clause 63. The method of any one of clauses 57-62, wherein the polymerization reaction is conducted with monomers including acrylate monomers.
   Clause 64. The method of any one of clauses 57-63, further comprising, while the polymer is polymerized or after the polymer is polymerized, cross-linking the polymer, forming a hydrogel comprising the extracellular vesicles, wherein the polymer optionally comprises a saturated carbon backbone and is functionalized with poly(ethylene oxide)-containing groups, and the cross-linker comprises poly(ethylene oxide) groups, the poly(ethylene oxide) groups having the structure —(O—CH₂—CH₂—)ₙ, where n optionally is 100 or less, 20 or less or 10 or less.
   Clause 65. The method of any one of clauses 36-64, wherein the extracellular vesicle comprises a biologically active agent, such as a therapeutic agent, such as a therapeutic loaded inside the lumen of the extracellular vesicle or on the membrane surface.
   Clause 66. The method of any one of clauses 36-65, wherein the hydrophobically-modified oligonucleotide is an oligonucleotide linked to a sterol, such as cholesterol, a GM1, a lipid, a vitamin, a small molecule, or a peptide, or a combination thereof.
   Clause 67. The method of any one of clauses 36-66, wherein the extracellular vesicles are exosomes.

### BRIEF DESCRIPTION OF THE DRAWINGS

FIG. 1: Schematic representation of Exosome binding to Anti-CD63 conjugated streptavidin beads.

FIG. 2: Gating strategy for flow cytometry analysis for experiment shown in FIGS. 14A-14B and FIGS. 15A-15B. Single cells from Spleen were analyzed by flow cytometry. Cells were first gated based on size (FSC-A vs SSCA) followed by doublets exclusion (FSC-H vs FSC-W and SSC-H vs SSC-W). Donor cells were discriminated from recipient on basis of H2Kd expression. Donor cells proliferation was analyzed as CFSE dilution on CD3, CD4 and CD8 population.

FIG. 3: Exosome-antibody Functionalization.

FIG. 4: Antibody tethering to exosomes. 5′-amine-DNA′ was functionalized with rabbit anti-human antibody (RAH) using the Solulink protein-oligo conjugation kit (Catalog S-9011-1, Solulink). Rabbit anti-human antibody-functionalized exosomes were prepared by preannealing approach using Chol-DNA and RAH-DNA′ strands. Figure shows the flow cytometry analysis of CD63 conjugated beads with RAHfunctionalized exosomes, followed by incubation with AF488-labeled Goat anti-rabbit antibody (GAR). Control experiment was performed by directly incubating the CD63 conjugated beads with AF488-labeled goat anti-rabbit antibody. A clear shift of fluorescence intensity in 488 nm channel verified the successful conjugation.

FIGS. 5A-5E: Functionalization of Exosomes using DNA tethers. (FIG. 5A) Schematic showing tethering of cholesterol-modified oligonucleotide to the membrane of an exosome. Cholesterol is present on 3′ chain end that anchors the single strand (SS)-oligonucleotide into the exosome membrane. A complimentary reporter strand can bind with the anchor strand resulting in a duplex oligonucleotide display on the exosome membrane and can incorporate additional functionality onto the modified exosome. A pre-annealed DNA strand with a cholesterol is present on 3′ chain end can be directly anchored to the exosome membrane in a simple vortex step at ambient temperatures. (FIG. 5B) Flow cytometric assessment of anchor DNA tethering on CD63 conjugated magnetic beads. DNA anchor concentration was varied between 0 to 50 μM and incubated with 20 μg of exosome which led to a corresponding increase in fluorescence intensity. (FIG. 5C) Bars indicate relative mean fluorescence intensities±SEM (n=3). (FIGS. 5D and 5E) Stability of single strand DNA (ssDNA) and double strand DNA (dsDNA) on exosome membrane assessed using on-bead flow cytometry at 4° and 37°. There was minimal loss in anchored oligonucleotide at 4° C., whereas up to 32.3(±) % stays bound to exosome membrane up to 7 days in simulated body fluid. Tethered oligonucleotides can be degraded using DNase-I. Bars indicate relative mean fluorescence intensities±SEM (n=3).

FIGS. 6A-6C: Characterization of exosomes. (FIG. 6A) Representative transmission electron micrograph (TEM) of THP1 and J774A.1 exosomes showing vesicles between 30 nm to 200 nm. (FIG. 6B) Tunable resistive pulse sensing analysis of THP1 exosomes showing mean diameter of 100 nm. (FIG. 6C) Western blot analysis for exosomal markers CD9, CD63 and TSG101.

FIG. 7: Representative confocal images of exosomes captured with CD63-conjugated beads. Chol-ssDNA-Cy5 and Chol-dsDNA-Cy5 were tethered to THP1 exosomes and captured with CD63 magnetic beads. Captured exosomes were pipetted onto a standard glass slide, allowed to dry and imaged using ZEISS LSM 880 confocal microscope under constant settings. Increasing concentration of membrane conjugated oligonucleotides resulted in a corresponding increase in fluorescence from the beads. Treating the beads with 2.5 Units of DNase-I for 15 min at 37° C. results in degradation of tethered oligonucleotides, subsequently decreasing the fluorescence from the magnetic beads. Scale bar=20 μm.

FIG. 8: Histogram representation of fluorescence from flow cytometry experiments evaluating ssDNA tethering to THP1 exosomes. Increasing concentration of Chol-DNA-Cy5 in the membrane resulted in corresponding increase in the Cy5 fluorescence intensity from the beads.

FIGS. 9A-9B: Optimization and characterization of dsDNA tethered exosomes. (FIG. 9A) Reporter strand (dsDNA) titration curve as evaluated on CD63 magnetic beads. Different concentrations of complementary DNA′ in the increment of 0.5×, 1×, 2× and 4× the concentration of tethered ssDNA were evaluated to optimize its concentration. 2× concentration of DNA′ resulted in a saturation of fluorescence signal from the magnetic beads and hence was chosen for subsequent experiments. Bars indicate mean±SEM (n=3), ****p<0.001, ns: no significant difference. (FIG. 9B) Evaluation of different annealing conditions for hybridization of Cy5-labeled complementary strand on ssDNA-tethered exosomes. Bars indicate mean±SEM (n=3), ****p<0.001 vs other two conditions.

FIGS. 10A-10B: Assessment of cell internalization of Exosome DNA hybrids. (FIG. 10A) To inhibit cellular uptake, cells were pretreated with a combination of 10 ug/ml heparin and 1 μM methyl-β-cyclodextrin for 1 hour at 37° C. Both native and oligonucleotide tethered exosomes behaved similar to native exosomes. There was a linear increase in exosome uptake from 3 to 6 hours, whereas in presence of inhibitors, the internalization was reduced to 30 (±) %. Bars represent relative mean fluorescence intensities±SEM (n=3). (FIG. 10B) Rescue of internalization inhibition in MIAPaCa2 cells by conjugating AS1411 aptamer onto exosome surface. AS1411 binds to cell surface nucleolin expressed only on MIAPaCa2 cells and is thereby able to overcome the inhibitory effects of heparin and mβCD.

FIG. 11: Quantification of native exosome and Exo-ssDNA-Cy5 internalization from confocal images of three independent experiments. Bars indicate mean±SEM (n=3).

FIGS. 12A-12C: In vivo assessment of SAFasL conjugated exosomes. (FIG. 12A) Schematic showing procedure for binding of surface bearing FasL to FasR on T-cells resulting in their apoptosis. (FIG. 12B) Dosage curve for exosome-FasL showing a dosage depended apoptosis in Jurkat cells as evaluated by flow cytometry. The dosage curve consisted of treatments with varying concentration of exosome containing 0.1 μM dsDNA-biotin with 100 ng of SAFasL. The lowest concentration i.e., 1 μg exosome protein resulted in 17.46 (±0.86) % apoptosis whereas 20 μg exosome protein resulted in 99.3 (±0.03) %. Non-conjugated 100 ng of soluble SAFasL or native exosomes did not result in any significant apoptosis. Bars indicate mean±SEM (n=3). (FIG. 12C) Titration of SAFasL-tethered exosomes on Jurkat cells showed a dose-dependent increase in apoptosis. The dosage consisted of varying concentrations of exosomes with 0.1 μM Chol-dsDNAbiotin with 100 ng of SA-FasL. The lowest concentration, i.e., 1 μg/mL exosome-dsDNA-SA-FasL, resulted in 17.46% (±0.86) apoptosis, whereas 20 μg of exosome-dsDNA-SA-FasL resulted in 99.3% (±0.03), while native exosomes, 100 ng of soluble SA-FasL, or ds-DNA-SAFasL did not result in any significant apoptosis. Bars indicate mean±SEM (n=3 independent experiments), ns: no significant difference, ***p=0.003, ****p<0.0001 vs soluble SA-FasL treatment.

FIG. 13A-13D: Images from bioprinting of Exosome-FasL. (FIG. 13A) Co-localization of Exosome and DNA, Exosome (PKH67): Green and DNA Tether (Cy5): Red. (FIG. 13B) Relative fluorescence intensity across the gradient deposited screen. (FIG. 13C) The normalized on-off pattern. (FIG. 13D) Fluorescence images show live/dead (calcien AM/ethidium bromide) staining of PCI-13 cells post 24 hours on bioprinted patterns of native exosomes, SA-FasL and Chol-ssDNA-SA-FasL tethered exosomes on collagen type-1 coated coverslips. Scale bar=600 μm.

FIGS. 14A-14B: Systemic delivery of SA-FasL-tethered exosomes blocks the proliferation of donor T cells in vivo. The percentages of donor CD3-positive T cells were assessed by gating on donor (H2Kd negative) cells (% CD3) in treatment and control groups in spleen (FIG. 14A) and mesenteric lymph nodes (FIG. 14B). The proliferation of donor CD3+, CD4+, and CD8+ T cells was measured by CFSE dilution using an LSR II and Diva software (BD Biosciences). ***p=0.0001, **p=0.003, and *p<0.05.

FIGS. 15A-15B: SA-FasL tethered exosomes blocked the proliferation of donor T cells in-vivo. Absolute cell number of donor CD3 positive T cells were calculated by gating on donor (H2Kd negative) cells (CD3) in treatment and control groups in Spleen (FIG. 15A) and Mesenteric Lymph node (FIG. 15B). The proliferation of donor CD3+, CD4+ and CD8+ T cells was measured by CFSE dilution using BD LSR II and Diva software (BD Biosciences). ***P=0.0001, **P=0.003 and *P<0.05.

FIGS. 16A-16C: Exosome functionalization using click chemistry. (FIG. 16A) Schematic showing click reaction of azide-functionalized exosomes with either fluorescent dyes or polyethylene glycol (PEG) under copper—catalyzed or Cu-free click conditions. (FIG. 16B) Flow cytometric analysis of click reaction of SF488-DBCO and Cyanine5-alkyne dye under Copper-free and Copper-catalyzed conditions respectively. (FIG. 16C) Dynamic light scattering analysis of exosomes functionalized with PEG₃₀ₖ polymer using Cu-free click reaction.

FIGS. 17A-17C: Grafting-to strategy for the preparation of exosome polymer hybrids. (FIG. 17A) Schematic of polymer functionalization of exosome membrane by grafting-to by annealing and preannealing approaches. In the annealing approach, a well-defined complementary DNA′-polymer can be annealed to Exo-ssDNA to prepare EPHs. Alternatively, in the preannealing approach the Chol-DNA and DNA′-polymer can be annealed before tethering to exosomes. Inset shows the structures of some of the polymer sidechains (FIG. 17B) Plot showing size and surface charge of EPHs prepared by both annealing and preannealing approach with varying loading (0 μM to 20 μM) of DNA′-pOEOMA_{30K}. (FIG. 17C) Graph shows size and surface charge of EPHs prepared with pOEOMA, pCBMA, and pMSEA by preannealing approach at 1 μM loading of polymers.

FIG. 18: Preparation of exosome polymer hybrids using DNA tethers. Schematic for the preparation of exosome polymer hybrids (EPHs). Cholesterol-modified DNA (Chol-DNA) tethers on the exosome membrane lead to Exo-ssDNA to which a complementary DNA block copolymer (DNA′-Polymer) can be used to prepare EPHs by ‘grafting-to’ strategy. Alternatively, in a ‘grafting-from’ strategy, a macroinitiator (DNA′-Initiator) can be hybridized to the DNA of Exo-ssDNA, followed by surface-initiated controlled radical polymerization.

FIGS. 19A-19C: Grafting-from strategy for the preparation of exosome polymer hybrids. (FIG. 19A) Schematic for the grafting of polymers directly from exosome surfaces by blue light-mediated photoATRP. An ATRP initiator directly on a DNA tether on the exosome lipid membrane initiates polymer chains to prepare homopolymers, which may even be subsequently chain extended to prepare block copolymers. (FIG. 19B) Plot showing size distribution of native exosomes and EPHs after synthesis of two polymer blocks of pOEOMA. (FIG. 19C) Plot showing size distribution of native exosomes and EPHs after grafting polymer block of pOEOMA and chain extension using pDMAEMA.

FIGS. 20A-20D: Analysis of surface accessibility of exosome polymer hybrids. (FIG. 20A) Schematic of the binding of the exosome surface protein CD63 on Cy5-labeled Exo-pOEOMA (Exo-pOEOMA-Cy5) to Anti-CD63 beads. The binding was evaluated by flow cytometry using varying polymer lengths (10K, 20K, 30K) and surface loadings (0-5 μM) of Exo-pOEOMA-Cy5 hybrids. Inset shows the influence of different polymer loadings (by varying the concentration of DNA′-pOEOMA) on the accessibility of the CD63 protein on the Exo-pOEOMA-Cy5 surface. (FIGS. 20B, 20C, 20D) Graphs of the mean fluorescence intensity (MFI) of anti-CD63 beads-bound Exo-pOEOMA-Cy5 with different lengths of pOEOMA—10K (FIG. 20B), 20K (FIG. 20C), 30K (FIG. 20D) and varying DNA′-polymer loadings. To assess the accessibility of DNA tethers, beads were also incubated with nuclease DNase I for 60 min at 37° C. The drop in the MFI post-nuclease treatment highlights the degradation of DNA-polymer strands off the exosomes. Bars indicate MFI±SEM (n=3).

FIGS. 21A-21C: Effect of polymer functionalization on the stability of exosomes. (FIG. 21A) EPHs can be reversibly functionalized with polymers using a DNA tether with a photocleavable (pc) p-nitrophenyl spacer incorporated (FIG. 21B) Plots showing the stability of exosomal surface proteins against trypsin by size exclusion chromatography. EPHs with pOEOMA and pCBMA, prepared using a pc DNA tether showed no degradation of surface proteins after incubation with trypsin at 37° C. for 1 h. After irradiation of EPHs with UV light (365 nm) for 2 min, removal of polymer from exosome surface showed protein degradation after 60 min. (FIG. 21C) Plot showing the change in the average diameter of native exosomes and Exo-pOEOMA (1 μM loading) after one month incubation at 4° C. and 37° C. in 1×PBS buffer. The study was repeated four times with each sample in triplicates.

FIGS. 22A-22H: Effect of polymer functionalization on the bioactivity of native and engineered exosomes in vitro. (FIG. 22A) Schematic diagram showing the in vitro assessment of bioactivity of native exosomes and engineered exosomes after polymer functionalization. (FIG. 22B) Plot comparing the cell internalization efficiency of native exosomes and EPHs with different length of pOEOMA polymer (1 μM loading) in HEK293 cells after 6 hours. (FIG. 22C) Plot showing the internalization efficiency of EPHs with different polymers in HEK293 cells after 6 hours. To inhibit two major pathways of exosome internalization, cells were treated with heparin and methyl-β-cyclodextrin. A drop in the cellular uptake of both native exosomes and EPHs highlights similar internalization mechanism. (FIG. 22D) Angiogenesis study using stem cell-derived exosomes and corresponding EPHs. (FIG. 22E) Osteogenesis studies using BMP2-loaded exosomes and corresponding Exo-pOEOMA hybrids. (FIG. 22F) Plot showing the angiogenesis property of stem cell-derived exosomes and Exo-pOEOMA (1 μM loading). (FIG. 33G) Plot showing the osteogenesis property of BMP2-loaded exosomes and Exo-pOEOMA (1 μM loading). (FIG. 22H) Plot showing the anti-inflammatory effects of curcumin-loaded exosomes and Exo-pOEOMA (1 μM loading). Similar activity was observed for native exosomes and EPHS.

FIGS. 23A-23B: Effect of polymer functionalization on the bioactivity of native and engineered exosomes in vivo. (FIG. 23A) Plot showing the fluorescent signal from native exosomes and exosome polymer hybrids in the blood at different time points. (FIG. 23B) Plot showing the percentage accumulation of exosome and exosome polymer hybrids in different organs of mice after 24 hrs.

FIG. 24: Schematic showing the formation of exosome-tethered and exosome-trapped gels by Atom Transfer Radical Polymerization. In addition to the PEO-based monomer, crosslinker and initiator, exosome macroinitiators are added to prepare exosome-tethered gels using oxygen tolerant blue light mediated photoATRP. Polymer chains growing from the exosome-tethered initiators crosslinks in the gel network and held by non-covalent cholesterol mediated interactions. Alternatively, preparation of gels in presence of non-functionalized native exosomes physically traps them in the gels.

FIG. 25: Plot showing the release kinetics of exosomes and BMP2 growth factor from the gel network. Trapped BMP2 and exosomes (native and BMP2-loaded) were cleared from the gel network in 1 day and 10 days respectively. On the contrary, exosome tethered to the gel network were retained even after 30 days. Exosomes with photocleavable tethers were irradiatiated with UV light for 2 mins, facilitating their complete removal from the gel network within next 24 hours.

FIG. 26: Figure showing the exosome gel-mediated osteogenic differentiation studies. Two bone formation markers—alkaline phosphatase (early marker) and mineral deposits (late marker) are assessed to highlight the superior bioactivity of BMP2-Exosome-tethered gel (BMP2-Exo-Gel). Similar to controls with liquid phase BMP2 and BMP2-loaded exosomes, ALP expression was upregulated by both BMP2-gel and BMP2-Exo-Gel in 72 hours. However, mineralization assay over a period of 28 days showed mineral deposits with BMP2-Exo-Gels while BMP2-Gel did not result in formation of mineral deposits. Controls were performed by supplementing 100 ng/ml BMP2 every 72 hours.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, a “patient” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). As used herein, the terms “treating”, or “treatment” refer to a beneficial or desired result, such as improving one of more functions, or symptoms of a disease.

“Therapeutically effective amount,” as used herein, is intended to include the amount of a recognition reagent as described herein that, when administered to a subject having a disease, is sufficient to effect treatment of the disease (e.g., by diminishing, ameliorating or maintaining the existing disease or one or more symptoms of disease). The “therapeutically effective amount” may vary depending on compound or composition, how it is administered, the disease and its severity and the history, age, weight, family history, genetic makeup, the types of preceding or concomitant treatments, if any, and other individual characteristics of the subject to be treated.

A “therapeutically-effective amount” also includes an amount of an agent that produces some desired local or systemic effect at a reasonable benefit/risk ratio applicable to any treatment. Compounds and compositions described herein may be administered in a sufficient amount to produce a reasonable benefit/risk ratio applicable to such treatment.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As used herein, the term “nucleic acid” refers to deoxyribonucleic acids (DNA) and ribonucleic acids (RNA). Nucleic acid analogs include, for example and without limitation: 2′-O-methyl-substituted RNA, locked nucleic acids, unlocked nucleic acids, triazole-linked DNA, peptide nucleic acids, morpholino oligomers, dideoxynucleotide oligomers, glycol nucleic acids, threose nucleic acids and combinations thereof including, optionally ribonucleotide or deoxyribonucleotide residue(s). Herein, “oligonucleotide” is a short, single-stranded structure made of up nucleotides, includes nucleic acids, nucleic acid analogs, or a chimera thereof, as oligonucleotides may include a combination of both standard nucleotide monomer residues and synthetic nucleotide monomer residues. An oligonucleotide may be referred to by the length (i.e., number of nucleotides) of the strand, through the nomenclature “-mer”. For example, an oligonucleotide of 22 nucleotides would be referred to as a 22-mer. An oligonucleotide comprises a sequence of nucleobases (“has a sequence of bases”, or simply “has a sequence”) that is able to hybridize to a complementary sequence on an oligonucleotide, a nucleic acid, or a nucleic acid analog by cooperative base pairing, e.g., Watson-Crick base pairing or Watson-Crick-like base pairing.

A “nucleic acid analog” is a composition comprising a sequence of nucleobases arranged on a substrate, such as a polymeric backbone, and can bind DNA and/or RNA by hybridization by Watson-Crick, or Watson-Crick-like hydrogen bond base pairing. Non-limiting examples of common nucleic acid analogs include peptide nucleic acids (PNAs), such as γPNA, morpholino nucleic acids, phosphorothioates, locked nucleic acid (2′-O-4′-C-methylene bridge, including oxy, thio or amino versions thereof), unlocked nucleic acid (the C2′-C3′ bond is cleaved), α, β-constrained nucleic acid, 2′-fluoro RNA, phosphorodiamidate morpholino, 2′-O-methyl-substituted RNA, threose nucleic acid, glycol nucleic acid, 2′,4′-constrained ethyl nucleic acid, 2′,4′ bridged nucleic acid NC (N—H), 2′,4′ bridged nucleic acid NC (N-methyl), ((S)-5′-C-methyl DNA (RNA)), and 5′-E-vinylphosphonate nucleic acid, among others. A “peptide nucleic acid” (PNA) refers to a nucleic acid analog, or DNA or RNA mimic, in which the sugar phosphodiester backbone of the DNA or RNA is replaced by an N-(2-aminoethyl)glycine unit. A gamma PNA (yPNA) is an oligomer or polymer of gamma-modified N-(2-aminoethyl)glycine monomers to produce a chiral center.

In the context of the present disclosure, a “nucleotide” refers to a monomer comprising at least one nucleobase and a backbone element (backbone moiety), which in a nucleic acid, such as RNA or DNA, is ribose or deoxyribose. “Nucleotides” also typically comprise reactive groups that permit polymerization under specific conditions. In natural DNA and RNA, those reactive groups are the 5′ phosphate and 3′ hydroxyl groups. For chemical synthesis of nucleic acids and analogs thereof, the bases and backbone monomers may contain modified groups, such as blocked amines, as are known in the art. A “nucleotide residue” refers to a single nucleotide that is incorporated into an oligonucleotide or polynucleotide. The backbone monomer can be any suitable nucleic acid backbone monomer, such as a ribose triphosphate or deoxyribose triphosphate, or a monomer of a nucleic acid analog, such as peptide nucleic acid (PNA), such as a gamma PNA (γPNA). The backbone monomer may be a ribose mono-, di-, or tri-phosphate or a deoxyribose mono-, di-, or tri-phosphate, such as a 5′ monophosphate, diphosphate, or triphosphate of ribose or deoxyribose. The backbone monomer includes both the structural “residue” component, such as the ribose in RNA, and any active groups that are modified in linking monomers together, such as the 5′ triphosphate and 3′ hydroxyl groups of a ribonucleotide, which are modified when polymerized into RNA to leave a phosphodiester linkage. Likewise for PNA, the C-terminal carboxyl and N-terminal amine active groups of the N-(2-aminoethyl)glycine backbone monomer are condensed during polymerization to leave a peptide (amide) bond.

Complementary refers to the ability of polynucleotides (nucleic acids) to hybridize to one another, forming inter-strand base pairs. Base pairs are formed by hydrogen bonding between nucleotide units in polynucleotide or polynucleotide analog strands that are typically in antiparallel orientation. Complementary polynucleotide strands can base pair (hybridize) in the Watson-Crick manner (e.g., A to T, A to U, C to G), or in any other manner that allows for the formation of duplexes. In RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by the nucleobase content of the strands, the presence of mismatches, the length of complementary sequences, salt concentration, temperature, with the melting temperature (Tm) lowering with shorter complementary sequences, increased mismatches, and increased stringency. Perfectly matched sequences are said to be “fully complementary”, though one sequence (e.g., a target sequence in an mRNA) may be longer than the other.

An “extracellular vesicle” (EV) is a double-layer phospholipid membrane vesicle known to be released by most cells. EVs may carry biologically active molecules that can traffic to local or distant targets and execute defined biological functions. EVs typically have a diameter of 10 nm and above. However, EVs may be classified by size, biogenetic pathways, and function. Common classification includes endosomal sorting complexes required for transport (ESCRT) protein-based formation of intraluminal vesicles within multivesicular bodies (MVBs) (“exosomes”), a pathway that is shared by viruses; formation by pinching off from the plasma membrane (“microvesicles”); and membrane disintegration (“apoptotic bodies”). (See, e.g., Margolis L, Sadovsky Y (2019) The biology of extracellular vesicles: The known unknowns. PLOS Biology 17(7): e3000363. https://doi.org/10.1371/journal.pbio.3000363; Li, S., Lin, Z., Jiang, X. et al. Exosomal cargo-loading and synthetic exosome-mimics as potential therapeutic tools. Acta Pharmacol Sin 39, 542-551 (2018). https://doi.org/10.1038/aps.2017.178). Although many compounds, compositions, and methods described herein may be described in the context of exosomes, unless specifically indicated to the contrary, those compounds, compositions, and methods may be considered applicable to other EV types. Extracellular vesicles include but are not limited to exomeres, exosomes, outer-membrane vesicles, matrix vesicles, micro-vesicles or apoptotic bodies. For purposes herein, unless otherwise indicated, to the contrary, extracellular vesicles, e.g., exosomes, may be prepared or obtained from any biological source, such as, without limitation, from any living organism that produces extracellular vesicles, from cells, tissue, or organ cultures, e.g., from mammals of mammalian cell culture. Non-limiting examples of sources for extracellular vesicles include primary cell culture, stem cell culture, progenitor cell culture, recombinant cell culture, dendritic cell culture, among others.

U.S. Pat. No. 10,513,710, incorporated herein by reference for its exemplary technical disclosure, describing exosomes, methods of preparing exosomes, and methods and reagents useful for producing exosomes decorated with hydrophobically modified nucleic acids that are RNA interference reagents. The oligonucleotides are hydrophobically modified by linking a hydrophobic moiety to the oligonucleotide as described therein. The hydrophobic moieties may be a sterol such as cholesterol, GM1, a lipid, a vitamin, a small molecule, or a peptide, or a combination thereof. The hydrophobically modified nucleic acids (hydrophobically modified nucleic acid cargo) are associated with the exosome, for example and without intent to be bound by this theory, by insertion of the hydrophobic moiety of the hydrophobically modified nucleic acid in the lipid bilayer of the exosome. Other publications describe anchoring hydrophobically-modified oligonucleotides in exosomes (Pi et al. “Nanoparticle orientation to control RNA loading and ligand display on extracellular vesicles for cancer regression”, Nature Nanotechnology, 2018, 13:82-89). Cholesterol TEG (triethylene glycol spacer), for linking to oligonucleotides, is commercially available, e.g., as Cholesterol-TEG phosphoramidite (Glen Research, Sterling, Va.)

A “moiety” (pl. “moieties”) is a part of a chemical compound, and includes groups, such as functional groups but can include any portion of a compound. As such, a nucleobase moiety is a nucleobase that is modified by attachment to another compound moiety, such as a polymer monomer, e.g., the nucleic acid or nucleic acid analog monomers described herein, or a polymer, such as a nucleic acid or nucleic acid analog as described herein.

“Alkyl” refers to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. “Substituted alkyl” refers to alkyl substituted at 1 or more, e.g., 1, 2, 3, 4, 5, or even 6 positions, which substituents are attached at any available atom to produce a stable compound, with substitution as described herein. “Optionally substituted alkyl” refers to alkyl or substituted alkyl. “Halogen,” “halide,” and “halo” refers to F, Cl, Br, and/or I. “Alkylene” and “substituted alkylene” refer to divalent alkyl and divalent substituted alkyl, respectively, including, without limitation, ethylene (—CH₂—CH₂—). “Optionally substituted alkylene” refers to alkylene or substituted alkylene.

“Alkene or alkenyl” refers to straight, branched chain, or cyclic hydrocarbyl groups including, e.g., from 2 to about 20 carbon atoms, such as, without limitation C₁₋₃, C₁₋₆, C₁₋₁₀ groups having one or more, e.g., 1, 2, 3, 4, or 5, carbon-to-carbon double bonds. “Substituted alkene” refers to alkene substituted at 1 or more, e.g., 1, 2, 3, 4, or 5 positions, which substituents are attached at any available atom to produce a stable compound, with substitution as described herein. “Optionally substituted alkene” refers to alkene or substituted alkene. Likewise, “alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

“PEG” refers to polyethylene glycol. “PEGylated” refers to a compound comprising a moiety, comprising two or more consecutive ethylene glycol moieties. Non-limiting examples of PEG moieties for PEGylation of a compound include, one or more blocks of a chain of from 2 to 100, or from 2 to 50 ethylene glycol moieties, such as —(O—CH₂—CH₂)ₙ—, —(CH₂—CH₂—O)ₙ—, or —(O—CH₂—CH₂)ₙ—OH, where n ranges from 2 to 50.

Various linking reactions may be utilized to link or conjugate a first molecule to a second molecule, such as in linking a biologically active agent, such as a therapeutic agent, a polymer, or a member of a binding pair to an oligonucleotide. In the context of the present disclosure, conjugates may be prepared by reacting a functional group on an oligonucleotide with a functional group or groups on the item to be conjugated to the oligonucleotide, such as a polymer, a member of a binding pair such as an antibody, or a therapeutic agent. The reaction of the functional groups may be a “click” reaction, such as, for example and without limitation, a Staudinger ligation, an azide-alkyne cycloaddition, a reaction of tetrazine with a trans-cyclooctene, a disulfide linking reaction, a thiol ene reaction, a hydrazine-aldehyde reaction, a hydrazine-ketone reaction, a hydroxyl amine-aldehyde reaction, a hydroxyl amine-ketone reaction or a Diels-Alder reaction.

“Click” reactions, for example, are described in U.S. Pat. No. 7,795,355 and/or Canalle, L., et al., “Polypeptide-polymer Bioconjugates, Chemical Society Reviews 39(1), 329-353 (2010), which are incorporated herein by reference for their technical disclosures. Such click reaction are suitable for reaction of other complexing agents hereof with one or more polymers. In general, “click” reactions are a group of high-yield chemical reactions that were collectively termed “click chemistry” reactions by Sharpless in a review of several small molecule click chemistry reactions. Kolb, H. C.; Finn, M. G.; Sharpless, K. B. Angew. Chemie, Interl. Ed. 40, 2004-2021 (2001), the disclosure of which is incorporated herein by reference. As used herein, a “click reaction” refers to a reliable, high-yield, and selective reaction having a thermodynamic driving force of greater than or equal to 20 kcal/mol. Click chemistry reactions may, for example, be used for synthesis of molecules comprising heteroatom links. One of the most frequently used click chemistry reactions involves cycloaddition between azides and alkynyl/alkynes to form the linkage comprising a substituted or unsubstituted 1,2,3-triazole. Certain click reactions may, for example, be performed in alcohol/water mixtures or in the absence of solvents and the products can be isolated in substantially quantitative yield.

Examples of suitable click reactions for use herein include, but are not limited to, Staudinger ligation, azide-alkyne cycloaddition (either strain promoted or copper(I) catalyzed), reaction of tetrazine with trans-cyclooctenes, disulfide linking reactions, thiolene reactions, hydrazine-aldehyde reactions, hydrazine-ketone reactions, hydroxyl amine-aldehyde reactions, hydroxyl amine-ketone reactions and Diels-Alder reactions. In such click reactions, one of the functional groups of the click reaction is on the complexing agent and the other of the functional groups of the click reaction is on the polymer. In a number of representative studies, p-RNA were prepared with azido groups that may be clicked with an alkyne moiety (which may or may not bear a cleavable linking group spacer with the polymer). Alternatively, p-RNA may be prepared with an alkyne group that may be clicked with an azido moiety of the polymer.

It is noted that click chemistry may or may not yield a cleavable bond by which a therapeutic agent may be releasably-linked to another compound to be complexed with the EVs as described herein. As such, where click chemistry cannot be used to link such biologically active agents, those agents may be linked in a different manner so as to yield a hydrolyzable bond such as an ester bond, or may be complexed with an antibody, oligonucleotide, or other suitable binding partner to the active agent, or the active agent may be associated, e.g., by adsorption or absorption, with the EV. Click chemistry linkages may best be used herein when the intended purpose of the linking is to strongly associate one molecule with another.

A “polymer composition” is a composition comprising one or more polymers. As a class, “polymers” includes, without limitation, homopolymers, heteropolymers, co-polymers, block polymers, block co-polymers and can be both natural and/or synthetic. Homopolymers contain one type of building block, or monomer, whereas copolymers contain more than one type of monomer. The term “(co)polymer” and like terms refer to either homopolymers or copolymers. A polymer may have any shape for the chain making up the backbone of the polymer, including, without limitation: linear, branched, networked, star, brush, comb, or dendritic shapes.

A polymer “comprises” or is “derived from” a stated monomer if that monomer is incorporated into the polymer. Thus, the incorporated monomer (monomer residue) that the polymer comprises is not the same as the monomer prior to incorporation into a polymer, in that at the very least, certain groups/moieties are missing and/or modified when incorporated into the polymer backbone. A polymer is said to comprise a specific type of linkage if that linkage is present in the polymer, such as, without limitation: ester, amide, carbonyl, ether, thioester, thioether, disulfide, sulfonyl, amine, carbonyl, or carbamate bonds. The polymer may be a homopolymer, a copolymer, and/or a polymeric blend.

A polymer may be prepared from and therefore may comprise, without limitation, one or more of the following ethylenically-unsaturated monomer residues: vinyl, styryl, or acrylate monomers. Non-limiting examples of such acrylate monomers include: (meth)acrylic acid (where the (meth) prefix collectively referring to both acrylic acid forms and methacrylic acid forms), laurel acrylate, PEG acrylates, such as methoxy-capped oligo(ethylene oxide) (meth)acrylate, such as methoxy-capped (ethylene oxide)_{8,9} (meth)acrylate, zwitterionic (meth)acrylates, such as betaine moiety-containing (meth)acrylates, DMSO-like (meth)acrylates, such as 2-(methylsulfinyl)C₁₋₆ alkyl acrylate, e.g., 2-(methylsulfinyl)ethyl acrylate fatty acid (meth)acrylates, such as lauryl acrylate or octadecyl methacrylate, dimethylaminoethyl methacrylate, 2-hydroxy C₁₋₆ alkyl (e.g., 2-hydroxyethyl) (meth)acrylates, 3-azidopropyl methacrylate, glycidyl (meth)acrylates, t-butyl acrylates, methyl methacrylate, n-butyl methacrylate, styrene, acrylonitrile, (meth)acrylamides, 4-vinyl pyridine, or dimethyl(1-ethoxycarbonyl)vinyl phosphate among others.

A “saturated carbon backbone” for a polymer refers to a polymer or polymer, polymer block, or polymer segment having an uninterrupted carbon-only backbone, such as are present in polyvinyl polymers or polymer segments. Polymers having saturated carbon backbones may be prepared using one or more ethylenically unsaturated monomers. A saturated carbon backbone may include linear, branched, or cyclic alkane segments. A segment of a polymer composition is a portion of a polymer comprising one or more monomer residues. A block of a block copolymer may be considered to be a segment. Polymer compositions with saturated carbon backbones may be prepared in any suitable manner, and may be formed by radical polymerization, by anionic polymerization, or by other methods as are broadly known.

Monomers useful in preparing polymers described herein, for example by radical polymerization methods such as controlled radical polymerization, ATRP, Reversible Addition—Fragmentation chain Transfer (RAFT) polymerization, Activators ReGenerated by Electron Transfer (ARGET) ATRP, Initiators for Continuous Activator Regeneration (ICAR) ATRP, supplemental activator and reducing agent atom transfer radical polymerization (SARA) ATRP, electrochemically-controlled ATRP (e-ATRP), and photoinduced ATRP, may comprise ethylenic unsaturation, as are broadly-known. Illustrative of such ethylenically unsaturated monomers include the alkenes, such as ethylenes, e.g., propene, butene, octene, or decene, though typically the alkenes have a terminal carbon-carbon unsaturation, such as propene; aryl alkenes, such as styrene or α-methylstyrene; vinyl esters such, as vinyl acetate, vinyl propionate; acrylic monomers, such as acrylic acid, methyl methacrylate, methylacrylate, 2-ethyl-hexyl-acrylate, acrylamide, or acrylonitrile; divinyl phenyls, such as divinyl benzene; vinyl naphthyls; alkadienes, such as 1,3-butadiene; isoprene, chloroprene, and the like; vinyl halides, such as vinyl chloride or vinyl fluoride; vinylidene halides, such as vinylidene bromide, vinylidene fluoride, or vinylidene chloride.

An acrylic polymer is a polymer comprising polymerized acrylate monomers (acrylates, or acrylate residues as integrated into the polymer). Acrylates are prop-2-enoates, and also may be referred to as acrylic acid derivatives or α,β unsaturated carbonyl compounds. Acrylates may be substituted in a variety of ways, such as by adding a methyl group to the a carbon, or by adding a functional group to the carbon of the carbonyl group, for example such as by including an amine or a substituted amine moiety to form an acrylamide, by including a PEG moiety to form poly(ethylene glycol) acrylate, by including a zwitterionic moiety, such as a carboxybetaine moiety, to form zwitterionic acrylate, such as a carboxybetaine acrylate, or by including a methylsulfinylalkyl moiety to form a methylsulfinylalkyl acrylate having dimethyl sulfoxide-like properties.

A polymer also may be prepared from and therefore may comprise, without limitation, one or more of the following monomer residues: glycolide, lactide, caprolactone, dioxanone, and trimethylene carbonate. In general, useful (co)polymers may comprise monomers derived from alpha-hydroxy acids including polylactide, poly(lactide-co-glycolide), poly(l-lactide-co-caprolactone), polyglycolic acid, poly(dl-lactide-co-glycolide), and poly(l-lactide-co-dl-lactide); monomers derived from esters including polyhydroxybutyrate, polyhydroxyvalerate, polydioxanone, and polyglactin; monomers derived from lactones including polycaprolactone; monomers derived from carbonates including polycarbonate, polyglyconate, poly(glycolide-co-trimethylene carbonate), and poly(glycolide-co-trimethylene carbonate-co-dioxanone); monomers joined through urethane linkages, including poly(ester urethane) urea (PEUU), poly(ether ester urethane)urea (PEEUU), poly(ester carbonate)urethane urea (PECUU), or poly(carbonate)urethane urea (PCUU). Examples of suitable polymers may include, but are not limited to, polyacrylate, polymethacrylates, polyacrylamides, polymethacrylamides, polypeptides, polystyrenes, polyethylene oxides (PEO), poly(organo)phosphazenes, poly-1-lysine, polyethyleneimine (PEI), poly-d,l-lactide-co-glycolide (PLGA), and poly(alkylcyanoacrylate).

The polymer or polymers may have a molecular weight (Mₙ) between approximately 1 kDa and 60 kDa or between approximately 1 kDa and 50 kDa. The polymer(s) may have a polydispersity index (PDI) (or dispersity (D)) between 1 and 2, between 1 and 1.5 or between 1 and 1.2, where PDI=M_{w}/Mₙ, where M_{w} is the weight average molecular weight and Mₙ is the number average molecular weight. Of note, PDI for a polymer may increase as the length of the polymer increases. That said, controlled radical polymerization methods, e.g., ATRP, yield low PDI values, in the range of 2.0 or less.

Polymer functionality may, for example, be linear or branched, and may include a poly(ethylene glycol) (PEG), a PEG-like group, an amine-bearing group (including primary, secondary, tertiary amine groups), a cationic group (which may generally be any cationic group—examples include a quaternary ammonium group, a guanidine group (guanidinium group), a phosphonium group or a sulfonium group), a dimethylsulfoxide-like (DMSO-like) group including methylsulfinyl-terminated alkyl groups, such as methylsulfinyl-terminated C₁-C₆ alkyl groups, or a zwitterionic group, such as a betaine, a reactive group for modification of polymer with, for example, small molecules (including, for example, dyes and targeting agents), a polymer, a biomolecule, or a biologically-active agent, such as a therapeutic agent, for example a peptide such as a cell-adhesion peptide, examples of which include IKVAV (SEQ ID NO: 1), RGD, RGDS (SEQ ID NO: 2), AGD, KQAGDV (SEQ ID NO: 3), VAPGVG (SEQ ID NO: 4), APGVGV (SEQ ID NO: 5), PGVGVA (SEQ ID NO: 6), VAP, GVGVA (SEQ ID NO: 7), VAPG (SEQ ID NO: 8), VGVAPG (SEQ ID NO: 9), VGVA (SEQ ID NO: 10), VAPGV (SEQ ID NO: 11) and GVAPGV (SEQ ID NO: 12), cytokine, or a growth factor, a polysaccharide, an oligonucleotide, a biologic active agent, or a small-molecule active agent.

In a number of embodiments, the polymer(s) is/are formed via controlled radical polymerization (CRP). The polymer(s) may, for example, be formed via atom transfer radical polymerization or activators generated by electron transfer atom transfer radical polymerization, such as by Activators ReGenerated by Electron Transfer (ARGET) ATRP, Initiators for Continuous Activator Regeneration (ICAR) ATRP, supplemental activator and reducing agent atom transfer radical polymerization (SARA) ATRP, electrochemically-controlled ATRP (e-ATRP), or photoinduced ATRP.

Polymer functionality may, for example, be linear or branched, and may include polyethylene glycol, a PEG-like group, amine bearing groups (including primary, secondary, tertiary amine groups), cationic groups (which may generally be any cationic group—examples include quaternary ammonium group, phosphonium group or sulfonium group), reactive groups for modification of polymer with, for example, small molecules (including, for example, dyes and targeting agents), polymers and biomolecules. Examples of suitable polymers include, but are not limited to, polyacrylate, polymethacrylates, polyacrylamides, polymethacrylamides, polypeptides, polystyrenes, polyethylene oxides (PEO), poly(organo)phosphazenes, poly-1-lysine, polyethyleneimine (PEI), poly-d,l-lactide-co-glycolide (PLGA), and poly(alkylcyanoacrylate).

Polymers suitable for use herein may, for example, be prepared via anionic polymerization, cationic polymerization, condensation polymerization, free radical polymerization and CRP. Controlled radical polymerization processes have been described by a number of workers (see, for example, Baker, S. L.; Kaupbayeva, B.; Lathwal, S.; Das, S. R.; Russell, A. J.; Matyjaszewski, K., “Atom Transfer Radical Polymerization for Biorelated Hybrid Materials”, Biomacromolecules, 2019, 20 (12):4272-4298 and Matyjaszewski, K., “Advanced Materials by Atom Transfer Radical Polymerization”, Advanced Materials, 2018, 30(23):1706441, among many other publications). The use of a CRP for the preparation of an oligo/polymeric material allows control over the molecular weight, molecular weight distribution of the (co)polymer, topology, composition and functionality of a polymeric material. The topology can be controlled, allowing the preparation of linear, star, graft or brush copolymers, formation of networks or dendritic or hyperbranched materials. Composition can be controlled to allow preparation of homopolymers, periodic copolymers, block copolymers, random copolymers, statistical copolymers, gradient copolymers, and graft copolymers. In a gradient copolymer, the gradient of compositional change of one or more comonomers units along a polymer segment can be controlled by controlling the instantaneous concentration of the monomer units in the copolymerization medium, for example. Molecular weight control is provided by a process having a substantially linear growth in molecular weight of the polymer with monomer conversion accompanied by essentially linear semilogarithmic kinetic plots for chain growth, in spite of any occurring terminations. Polymers from controlled polymerization processes typically have molecular weight distributions, characterized by the polydispersity index of less than or equal to 2. Polymers produced by controlled polymerization processes may also have a PDI of less than 1.5, less than 1.3, or even less than 1.2.

In CRP, further functionality may be readily placed on the oligo/polymer structure including side-functional groups, end-functional groups or can comprise site specific functional groups, or multifunctional groups distributed as desired within the structure. The functionality can be dispersed functionality or can comprise functional segments. The composition of the polymer may comprise a wide range of radically (co)polymerizable monomers, thereby allowing the properties of the polymer to be tailored to the application. Materials prepared by other processes can be incorporated into the final structure.

In general, polymerization processes performed under controlled polymerization conditions achieve the above-described properties by consuming the initiator early in the polymerization process and, in at least one embodiment of controlled polymerization, an exchange between an active growing chain and dormant polymer chain that is equivalent to or faster than the propagation of the polymer. In general, CRP process is a process performed under controlled polymerization conditions with a chain growth process by a radical mechanism, such as, but not limited to; atom transfer radical polymerization (ATRP), stable free radical polymerization (SFRP), specifically, nitroxide mediated polymerization (NMP), reversible addition-fragmentation transfer (RAFT), degenerative transfer (DT), and catalytic chain transfer (CCT) radical systems. A feature of controlled radical polymerizations is the existence of equilibrium between active and dormant species. The exchange between the active and dormant species provides a slow chain growth relative to conventional radical polymerization, all polymer chains grow at the same rate, although overall rate of conversion can be comparable since often many more chains are growing. Typically, the concentration of radicals is maintained low enough to minimize termination reactions. This exchange, under appropriate conditions, also allows the quantitative initiation early in the process necessary for synthesizing polymers with special architecture and functionality. CRP processes may not eliminate the chain-breaking reactions; however, the fraction of chain-breaking reactions is significantly reduced from conventional polymerization processes and may comprise only 1-10% of all chains.

ATRP is one of the most robust CRP and a large number of monomers can be polymerized providing compositionally homogeneous well-defined polymers having predictable molecular weights, narrow polydispersity, and high degree of end-functionalization. Matyjaszewski and coworkers disclosed ATRP, and a number of improvements in the basic ATRP process, in a number of patents and patent applications. See, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,627,314; 6,790,919; 7,019,082; 7,049,373; 7,064,166; 7,157,530 and U.S. patent application Ser. No. 09/534,827; PCT/US04/09905; PCT/US05/007,264; PCT/US05/007,265; PCT/US06/33152 and PCT/US2006/048656, the disclosures of which are herein incorporated by reference.

The ATRP process can be described generally as comprising: polymerizing one or more radically polymerizable monomers in the presence of an initiating system; forming a polymer; and isolating the formed polymer. The initiating system comprises: an initiator having a radically transferable atom or group; a transition metal compound, i.e., a catalyst, which participates in a reversible redox cycle with the initiator; and a ligand, which coordinates with the transition metal compound. The ATRP process is described in further detail in international patent publication WO 97/18247 and U.S. Pat. Nos. 5,763,548 and 5,789,487.

An ATRP initiator may be any initiator suitable for initiating an ATRP polymerization reaction in the context of the methods described herein. A suitable ATRP initiator may be a group comprising an alkyl halide, such as an alkyl bromide or alkyl chloride, such as an α-bromoisobutyrate (iBBr) group, for photoinitiation. Other suitable initiators, such as α-functionalized ATRP initiators, are broadly-known, and initiators can be selected or designed to best balance polymer structure and polymerization kinetics.

A “functional group” or a “reactive group” is a reactive chemical moiety that can be used to covalently link a chemical compound to another chemical compound, such as include, for example and without limitation: hydroxyl, carbonyl, carboxyl, methoxycarbonyl, sulfonyl, thiol, amine, or sulfonamide.

Association of one molecule with another may be covalent or non-covalent. By attaching or linking one moiety to another, it is meant the linkage is covalent, as in, for example, polymerization, cross-linking, click chemistry reactions, or linking reactions using linkers. Complexing two molecules refers to a non-covalent association, such as by Van der Waals forces, hydrogen bonding, pi stacking, or ionic interactions. Hybridization of two complementary oligonucleotides, nucleic acids, and/or nucleic acid analogs, is a form of complexing, as used herein.

In the context of recognition reagents, the term “ligand” refers to a binding moiety for a specific target, its binding partner. Collectively the ligand and its binding partner are termed a binding pair, and in context of a binding pair, the ligand is referred to herein as a binding partner to avoid confusion with ligands for use in polymerization reactions. A binding partner can be a cognate receptor, a protein, a small molecule, a hapten, or any other relevant molecule, such as an affibody or a paratope-containing molecule. One common, and non-limiting example of a binding pair is streptavidin/avidin and biotin. The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen, which can be virtually any molecule. Antibody mimetics are not antibodies, but comprise binding moieties or structures, e.g., paratopes, and include, for example, and without limitation: an affibody, an aptamer, an affilin, an affimer, an affitin, an alphabody, an aticalin, an avimer, a DARPin, a funomer, a Kunitz domain peptide, a monobody, a nanoclamp, or other engineered protein ligands, e.g., comprising a paratope targeting any suitable epitope present in a sample.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, Fv, Fd, dsFv, scFv, diabody, triabody, tetrabody, di-scFv (dimeric single-chain variable fragment), bi-specific T-cell engager (BiTE), single-domain antibody (sdAb), or antibody binding domain fragments. In the context of targeting ligands, the antibody fragment may be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Ligands also include other engineered binding reagents, such as affibodies and designed ankyrin repeat proteins (DARPins), that exploit the modular nature of repeat proteins (Forrer T, Stumpp M T, Binz H K, Plückthun A: A novel strategy to design binding molecules harnessing the modular nature of repeat proteins, FEBS Lett 2003, 539: 2-6; Gebauer A, Skerra A: Engineered protein scaffolds as next-generation antibody therapeutics, Curr Opin Chem Biol 2009, 13:245-255), comprising, often as a single chain, one or more antigen-binding or epitope-binding sequences and at a minimum any other amino acid sequences needed to ensure appropriate specificity, delivery, and stability of the composition (see also, e.g., Nelson, A L, “Antibody Fragments Hope and Hype” (2010) MAbs 2(1):77-83).

As used herein, the terms “cell” and “cells” refer to any types of cells from any animal, such as, without limitation, rat, mice, monkey, and human. For example and without limitation, cells can be progenitor cells, such as stem cells, or differentiated cells, such as endothelial cells, smooth muscle cells. In certain embodiments, cells for medical procedures can be obtained from the patient for autologous procedures or from other donors for allogeneic procedures.

Extracellular vesicles may be loaded with any compatible biologically-active agent, such as a therapeutic agent by any useful method. Non-limiting examples of drug loading include passive or active absorption or adsorption, electroporation, and membrane-association with hydrophobic agents or agents comprising hydrophobic moieties (see, e.g., Olivier G. de Jong, Sander A. A. Kooijmans, Daniel E. Murphy, Linglei Jiang, Martijn J. W. Evers, Joost P. G. Sluijter, Pieter Vader, and Raymond M. Schiffelers, Drug Delivery with Extracellular Vesicles: From Imagination to Innovation. Accounts of Chemical Research 2019 52 (7), 1761-1770 doi:10.1021/acs.accounts.9b00109 and Lamichhane T N, Jay S M. Production of Extracellular Vesicles Loaded with Therapeutic Cargo. Methods Mol Biol. 2018; 1831:37-47. doi:10.1007/978-1-4939-8661-3_4).

The biologically active agent or therapeutic agent may, for example, be a partially or fully complementary strand of RNA, DNA, PNA or chimera. In a number of embodiments, the biologically active agent is a partially or fully complementary strand of guide RNA, siRNA, or any useful reagent for RNA interference or antisense methods. The biologically-active agent may be a recombinant genetic construct for expression of a gene and/or for introduction into, or modification of the genome of the target cell.

One or more therapeutic agents that may be complexed with the tethered EVs, linked to the described oligonucleotides, otherwise incorporated into the compositions described herein include, without limitation, anti-inflammatories, such as, without limitation, NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, anti-inflammatory cytokines, and anti-inflammatory proteins or steroidal anti-inflammatory agents); antibiotics; anticlotting factors such as heparin, Pebac, enoxaprin, aspirin, hirudin, plavix, bivalirudin, prasugrel, idraparinux, warfarin, coumadin, clopidogrel, PPACK, GGACK, tissue plasminogen activator, urokinase, and streptokinase; growth factors. Therapeutic agents include, without limitation: (1) immunosuppressants; glucocorticoids such as hydrocortisone, betamethisone, dexamethasone, flumethasone, isoflupredone, methylpred-nisolone, prednisone, prednisolone, and triamcinolone acetonide; (2) antiangiogenics such as fluorouracil, paclitaxel, doxorubicin, cisplatin, methotrexate, cyclophosphamide, etoposide, pegaptanib, lucentis, tryptophanyl-tRNA synthetase, retaane, CA4P, AdPEDF, VEGF-TRAP-EYE, AG-103958, Avastin, JSM6427, TG100801, ATG3, OT-551, endostatin, thalidomide, becacizumab, neovastat; (3) antiproliferatives such as sirolimus, paclitaxel, perillyl alcohol, farnesyl transferase inhibitors, FPTIII, L744, antiproliferative factor, Van 10/4, 5-FU, Daunomycin, Mitomycin, dexamethasone, azathioprine, chlorambucil, methotrexate, mofetil, vasoactive intestinal polypeptide, and PACAP; (4) antibodies; (5) drugs acting on immunophilins, such as cyclosporine, zotarolimus, everolimus, tacrolimus and sirolimus (rapamycin), interferons, TNF binding proteins; (6) taxanes, such as docetaxel; statins, such as atorvastatin, lovastatin, simvastatin, pravastatin, fluvastatin and rosuvastatin; (7) nitric oxide donors or precursors, such as, without limitation, Angeli's Salt, L-Arginine, Free Base, Diethylamine NONOate, Diethylamine NONOate/AM, Glyco-SNAP-1, Glyco-SNAP-2, S-Nitroso-N-acetylpenicillamine, S-Nitrosoglutathione, NOC-5, NOC-7, NOC-9, NOC-12, NOC-18, NOR-1, NOR-3, SIN-1, Sodium Nitroprusside, Dihydrate, Spermine NONOate, Streptozotocin; and (8) antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscarnet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulphate, Zn-pyrithione, ciprofloxacin, norfloxacin, afloxacin, levofloxacin, gentamicin, tobramycin, neomycin, erythromycin, trimethoprim sulphate, polymixin B and silver salts such as chloride, bromide, iodide and periodate.

Any useful cytokine or chemoattractant can be associated with any composition as described herein. For example and without limitation, useful components include growth factors, interferons, interleukins, chemokines, monokines, hormones, and angiogenic factors. In certain non-limiting aspects, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc., Mountain View, Calif.; ProSpec-Tany TechnoGene Ltd., Rehovot, Israel; and Cell Sciences®, Canton, Mass.

The therapeutic agent may be an angiogenic therapeutic agent, such as: erythropoietin (EPO), basic fibroblast growth factor (bFGF), acidic fibroblast growth factor (aFGF), fibroblast growth factor-2 (FGF-2), granulocyte colony stimulating factor (G-CSF), granulocyte macrophage colony stimulating factor (GM-CSF), hepatocyte growth factor (HGF), insulin-like growth factors 1 and 2 (IGF-1 and IGF-2), placental growth factor (PIGF), platelet derived growth factor (PDGF), stromal derived factor 1 alpha (SDF-1 alpha), vascular endothelial growth factor (VEGF), angiopoietins (Ang 1 and Ang 2), matrix metalloproteinase (MMP), delta-like ligand 4 (DII4), and class 3 semaphorins (SEMA3s), all of which are broadly-known, and are available from commercial sources.

The therapeutic agent may be an antimicrobial agent, such as, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

The therapeutic agent may be an anti-inflammatory agent, such as, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin. Other drugs that may promote wound healing and/or tissue regeneration may also be included.

The therapeutic agent may be an, such as: Macugen (pegaptanib sodium); Lucentis; Tryptophanyl-tRNA synthetase (TrpRS); AdPEDF; VEGF TRAP-EYE; AG-013958; Avastin (bevacizumab); JSM6427; TG100801; ATG3; Perceiva (originally sirolimus or rapamycin); E10030, ARC1905 and colociximab (Ophthotech) and Endostatin. Ranibizumab is currently the standard in the United States for treatment of neovascular AMD. It binds and inhibits all isoforms of VEGF. Although effective in many cases, treatment with ranibizumab requires sustained treatment regimens and frequent intravitreal injections. VEGF Trap is a receptor decoy that targets VEGF with higher affinity than ranibizumab and other currently available anti-VEGF agents. Blocking of VEGF effects by inhibition of the tyrosine kinase cascade downstream from the VEGF receptor also shows promise, and includes such therapies as vatalanib, TG100801, pazopanib, AG013958 and AL39324. Small interfering RNA technology-based therapies have been designed to downregulate the production of VEGF (bevasiranib) or VEGF receptors (AGN211745). Other potential therapies include pigment epithelium-derived factor-based therapies, nicotinic acetylcholine receptor antagonists, integrin antagonists and sirolimus. (See, e.g., Chappelow, A V, et al. Neovascular age-related macular degeneration: potential therapies, Drugs. 2008; 68(8):1029-36 and Barakat M R, et al. VEGF inhibitors for the treatment of neovascular age-related macular degeneration, Expert Opin Investig Drugs. 2009 May; 18(5):637-46.

Extracellular vesicles may be loaded with an appropriate, e.g., and effective amount of a therapeutic agent in any manner, such as by absorption or absorbance. Protein or nucleic acid therapeutic agents, such as biological drugs, therapeutic RNAs, or genetic constructs containing a gene for expression in a target cell or organism, may be produced in cell culture, e.g., in recombinant cells expressing a gene or producing an mRNA, or a recombinant viral genome, and extracellular vesicles produced by the cells including the therapeutic agent may be used in the methods and tethered extracellular vesicles as described herein.

As described above, according to one aspect of the invention, a tethered extracellular vesicle is provided, comprising an extracellular vesicle; a hydrophobically-modified first oligonucleotide anchored to the extracellular vesicle; and a second oligonucleotide hybridized to the first oligonucleotide linked to a member of a binding pair, a therapeutic agent, a surface, or a polymer. According to another aspect, a tethered extracellular vesicle is provided comprising: an extracellular vesicle; a hydrophobically-modified oligonucleotide anchored to the extracellular vesicle and linked to a polymer. The tethered EV compositions may be associated with a therapeutic agent that can be incorporated into or onto the EV, tethered to the EV by an oligonucleotide, or bound to a binding partner tethered to the surface of the EV an oligonucleotide or in any suitable manner. A polymer, as described herein, may be linked to a hydrophobically-modified oligonucleotide, or linked to the second oligonucleotide which, in turn, is hybridized to a hydrophobically-modified oligonucleotide anchored in the EV. The complexed polymer may be cross-linked with polymer chains of other tethered EVs to produce a hydrogel in which the EVs are tethered. Where a therapeutic agent is associated with the tethered EVs or the cross-linked polymer, the hydrogel composition will release the therapeutic agent in a sustained or delayed manner, depending on the physical and chemical features of the therapeutic agent, the composition of the cross-linked hydrogel, and the manner of which the therapeutic agent is associated with the EVs in the hydrogel. In one example, as below, a PEGylated acrylic polymer is tethered to EVs and is cross-linked with oligo(ethylene glycol) linkers, to form a hydrogel.

The tethered EV compositions described herein may be tethered to a surface, by conjugating the second oligonucleotide, or cross-linking the tethered polymer to a surface. The second oligonucleotide may be conjugated to a surface and then hybridized to the hydrophobically-modified oligonucleotide anchored in an EV. A surface complex, e.g., bead, may be used to purify or enrich vesicles, optionally followed by elution of the EV's from the second oligonucleotide, and subsequent complexing of the eluted EVs with another second oligonucleotide, e.g., for associated with a therapeutic agent, and/or addition or, or grafting of a polymer and incorporation into a hydrogel as described herein.

Examples of suitable surfaces include, without limitation, plastics or polymeric surfaces, silicon wafers or chips, glass, ceramics, metals, beads, and porous matrices. Two or more different EVs may be localized at different, discretely addressable locations on a surface to produce an array or pattern on the surface. An EV may be complexed with a magnetic bead for magnetic sorting or purification. An EV may be complexed with a fluorescently-labeled bead for flow sorting, as with flow cytometry, and different EVs may be complexed with differently-labeled beads for sorting or analytical purposes. An EV may be complexed with a bead, such as an agarose bead or onto a porous matrix for affinity purification or for analytical methods. As would be recognized by those of ordinary skill, EVs may be complexed with members of binding pairs, such as antibodies, for use in analytical methods, such as sandwich-type assays, competition assays, or other analytical methods that might require an EV.

EVs may be complexed with a surface, such as a tissue culture plate or vessel, to produce a layer of EVs that produce any desired biological effect in cells cultured on or with the surface-bound EVs. This may be used for analytical purposes, for example as shown in the ExoFasL example below. EVs complexed with a surface also may be used as a coating for cell growth surfaces in cell culture vessels, such as bioreactors, to modify cell growth, cell differentiation, cell activity, or any other activity of the cells. For example, the surface-bound (e.g., bioprinted) ExoFasL EVs may be used to prevent cell growth on certain parts of, or areas of a bioreactor.

A bead may be complexed with an extracellular vesicle. Beads may be magnetic beads, agarose beads, polymeric beads, fluorescently-labeled beads, beads labeled with quantum dots, or any suitable beads, as are broadly-known in the arts. Beads may be complexed with an EV in any manner. As a non-limiting example, a bead having surface-bound streptavidin, as are broadly-available, may be complexed with a biotinylated oligonucleotide, which, in turn is hybridized to a hydrophobically-modified complementary oligonucleotide associated with an EV.

Also provided herein are methods of making tethered EV compositions. The method may comprise anchoring a hydrophobically-modified oligonucleotide to an extracellular vesicle; hybridizing to the hydrophobically-modified oligonucleotide a second oligonucleotide complementary to the hydrophobically-modified oligonucleotide and linked to a member of a binding pair, a therapeutic agent, a surface, a polymer initiator group, or a polymer. The method may comprise, anchoring a hydrophobically-modified oligonucleotide comprising a polymer initiator group to the extracellular vesicle; and polymerizing a polymer in a polymerization reaction from the polymer initiator group.

### Example 1: Oligonucleotide Tethering

The development of an alternative novel strategy to modify the external properties of exosomes with bioactive proteins is disclosed. The procedure provides a rapid and highly versatile method for exosome functionalization through a controlled membrane engineering approach. The hydrophobic interior of the lipid bilayer of an exosome selectively anchors an oligonucleotide through interaction with molecules incorporating small hydrophobic groups, like cholesterol, tocopherol, or sterol, on one of the chain ends predominately to the exterior of the membrane. This oligonucleotide-based anchoring strategy provides easy functionalization, tailored modification, and reversibility. In one exemplary example, a membrane anchored single-stranded DNA oligonucleotide acts as a “handle” and DNA complementarity can be exploited to attach small molecules, dyes, proteins or incorporate chemical functionality for further functionalization and modification of the anchored extracellular oligonucleotide. The disclosed procedure can be applied to engineer the surface of all natural and synthetic exosomes, liposomes, extracellular membranes, in addition to prokaryotic cells and eukaryotic cells.

This approach preserves the native cell-exosome binding interactions as assessed by on-bead flow cytometry and confocal microscopy based internalization studies. Moreover, the exemplified process demonstrates that exosomes can be engineered to carry native bioactive cargo capable of altering the physiology of recipient cells.

### Materials and Methods

Cell Culture: THP1 cells (ATTC TIB202) and J774A.1 cells (ATTC TIB-67) were cultured in heat-inactivated fetal bovine serum (HI-FBS; ThermoFisher Scientific, Waltham, Mass.) that had been depleted of exosomes. HI-FBS was centrifuged at 100,000×g for 3 hours and the exosome depleted supernatant was collected (ED-HI-FBS). The final media for THP1 cells consisted of RPMI-1640 (ThermoFisher Scientific, Waltham, Mass.) supplemented with 10% ED-HI-FBS and 1% Penicillin-Streptomycin (PS; ThermoFisher Scientific, Waltham, Mass.). Jurkat cells (ATTC TIb-152) were grown in RPMI-1640 supplemented with 10% ED-HI-FBS and 1% PS. HEK293, MIAPaCa2 and PCI13 cells were cultured and maintained in Delbecco's modified eagle media (DMEM; ThermoFisher, Waltham, Mass.) supplemented with 10% ED-HI-FBS and 1% PS. All the cell lines were regularly tested for mycoplasma contamination and were negative. Additionally, J774A.1 cells used for in vivo studies were certified by IDEXX BioResearch (Columbia, Mo.) to be free of bacteria, virus, and mycoplasma.

Exosome Isolation and Characterization: Exosomes were isolated from THP1 cells using the mini-SEC method as previously described (Hong et al. “Circulating exosomes carrying an immunosuppressive cargo interfere with cellular immunotherapy in acute myeloid leukemia”, Scientific reports, 2017, 7(1):14684). Briefly, conditioned media (minimum of 48 hours in cell culture) were differentially centrifuged (2500×g for 10 min at 4° C. and 10,000×g for 30 min at 4° C.), followed by ultrafiltration (0.22 μm filter; Millipore-Sigma, Billicera, Mass.) and then size-exclusion chromatography on an A50 cm column (Bio-Rad Laboratories, Hercules, Calif.) packed with Sepharose 2B (Sigma-Aldrich, St. Louis, Mo.). Protein concentrations of exosome fractions were determined using a BCA Protein Assay kit as recommended by the manufacturer (Pierce, ThermoFisher Scientific, Waltham, Mass.). Further characterization of exosomes was done with dynamic light scattering (DLS), tunable resistive pulse sending (TRPS), western blotting, Nanoparticle tracking analysis (NTA) and transmission electron microscopy (TEM).

Dynamic Light Scattering (DLS) and Zeta potential studies: Size measurements of exosomes were carried out using a Zetasizer (Malvern Instruments Ltd, England, UK). Exosomes diluted in phosphate buffered saline (PBS) (1:100) were analyzed in equilibration time of 120 seconds (sec) at the constant temperature of 25 degrees Celsius (° C.). Zeta potential was recorded using folded capillary zeta cells (Malvern Instruments Ltd, England, UK) in PBS buffer at room temperature.

Nanoparticle Tracking Analysis (NTA): Exosomes were diluted to an appropriate level with particle-free PBS and continuously fed into the Nanoparticle Tracking Analysis system (NTA; Nanosight, Amesbury, UK) LM-10 system with a syringe pump. The Brownian motion of each individual exosomes within the field of view was visualized with a laser illumination unit and a high-definition CCD camera. Each measurement was recorded for 1 minute (min) and repeated for three times. The size distribution of exosomes was then analyzed and extracted from the motion of exosomes using the software that came with the NTA system.

Tunable Resistive Pulse Sensing: TRPS system by qNano (Izon, Cambridge, Mass., USA) was used to measure the size distribution and concentration of particles in isolated exosome fractions as previously described (Yernani et al.). 40 microliters (μl) exosome suspension or calibration particles included in the reagent kit (2:1, 114 nanometers (nm), Izon) were placed in the Nanopore (NP100 #A28126, Izon). All samples were measured at 45.06 millimeters (mm) stretch at 0.64 volts (V) and 11 millibar (mbar) pressure. Particles were detected in short pulses of the current (blockades). The calibration particles were measured directly before and after the experimental sample under identical conditions. The sizes and concentrations of particles were determined using software provided by Izon (version 3.2).

Transmission Electron Microscopy: (Electron Microscopy Services, Hatfield, Pa.) Isolated total exosomes were fixed with 4% glutaraldehyde for 20 min at room temperature (RT). A 10 μL droplet of glutaraldehyde-fixed exosomes was placed on fomvar-coated 300 mesh copper grid. The sample was incubated for 1 min followed by rinsing with Deionized (DI) water for 1 min to ensure removal of PBS salts. Excess liquid was blotted-off with Whatman filter. Post rinsing, 50 μl of Uranyl-acetate solution was put on the grid and allowed to remain for 1 min. Excess liquid was removed, and the grids were viewed on a Hitachi H-7100 transmission electron microscope (TEM; Hitachi High Technologies) operating at 100 kiloeletron volts (keV). Digital images were collected using an AMT Advantage 10 CCD Camera System (Advanced Microscopy Techniques) and inspected using NIH ImageJ software.

Western blotting: Western blots for exosome proteins was performed as previously described (Yerneni et al.). Briefly, exosomes (10 micrograms (μg) protein after concentration of the collected 1 milliliter (mL) fractions by VivaSpin 500 or 50 μL of each fraction) were lysed with Laemmli sample buffer (Bio-Rad Laboratories, Hercules, Calif., USA), separated on 7-15% sodium dodecyl sulfate-polyacrylamide gel electrophoresis (SDS/PAGE) gels and transferred onto the polyvinylidene fluoride (PVDF) membrane (Millipore, Billerica, Mass., USA) for western blot analysis. Membranes were incubated overnight at 4° C. with TSG101 antibody (1:500; catalog #MA1-23296, ThermoFisher Scientific, Waltham, Mass.). Next, the horseradish peroxidase (HRP)-conjugated secondary antibody (1:5,000, Pierce, ThermoFisher Scientific, Waltham, Mass.) was added for 1 hour (hr) at room temperature (RT), and blots were developed with ECL detection reagents (GE Healthcare Biosciences, Marlborough, Mass.).

DNA Synthesis: All DNA sequences were synthesized using MerMade4 DNA synthesizer (Bioautomation, Irving, Tex.) using the standard DNA phosphoramidites (Chemgenes, Wilmington, Mass.). Chol-DNA sequences were prepared by coupling Spacer9 and Cholesterol-TEG phosphoramidites (Glen Research, Sterling, Va.) on the 5′-end. Cyanine5 (Cy5) labeled DNA strand was synthesized using Cyanine5 CPG beads (Glen Research, Sterling, Va.) and spacer9 and Cholesterol-TEG phosphoramidite were coupled on the 5′-end. Additionally, a photocleavable DNA tether (Chol-pc-DNA) was synthesized by coupling a p-nitrophenyl-based PC Linker phosphoramidite (Glen Research, Sterling, Va.) on 5′-end post DNA synthesis, followed by coupling with spacer9 and Cholesterol-TEG phosphoramidites.

After synthesis, DNA sequences were cleaved and deprotected from CPG beads and purified by reverse phase high pressure liquid chromatography (HPLC) using a C18 column. The eluent was 100 mM (millimolar) triethylamine-acetic acid buffer (TEAA, pH 7.5) and acetonitrile (0-30 min, 10-100%). All DNA concentrations were characterized with Nanodrop instrument (ThermoFisher Scientific Inc., Waltham, Mass.). Mass spectrometry of DNA sequences was performed using an Applied Biosystems Voyager DE-STR matrix assisted laser desorption/ionization time-of-flight (MALDI-TOF) instrument in positive mode with a 3-hydroxypicolinic acid matrix.

DNA tethers for exosomes: 20 μg of isolated THP1 exosomes were gently vortexed (600 revolutions per minute (RPM), Scientific Industries Vortex-Genie 2 Vortex Mixer) with different concentrations of cholesterol-DNA (Chol-DNA; 0.1 μM to 20 μM) for 5 min at room temperature in 1004 PBS buffer (final exosome concentration=0.2 μg/μL). Samples were then washed with Amicon Ultra Centrifugal Filters (100 k MWCO, Millipore Sigma, St. Louis, Mo.), followed by reverse spin to get ssDNA-tethered exosomes (Exo-ssDNA).

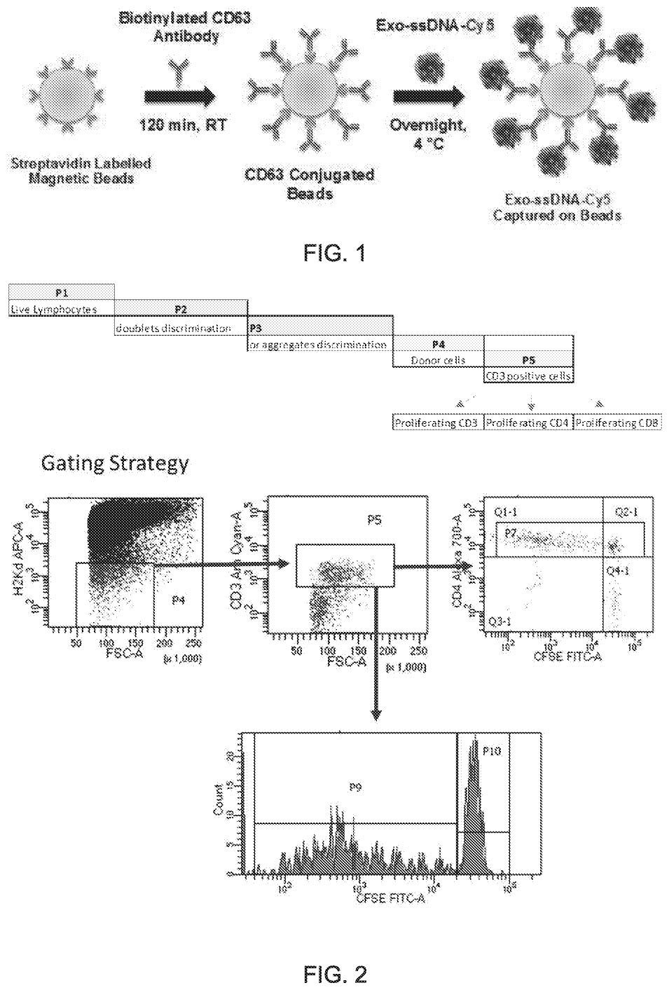

Flow Cytometry studies: Exo-ssDNA-Cy5 were prepared using Cy5-conjugated Chol-DNA (Cholesterol and Cy5 on the 5′ and 3′ end respectively) using the tethering protocol mentioned above. Exo-ssDNA-Cy5 (6 μg protein) were gently vortexed overnight at 4° C. with anti-CD63 conjugated magnetic streptavidin beads as shown in FIG. 1. For the control experiments, beads were incubated with the Chol-DNA-Cy5 to determine non-specific binding. Beads were washed with 1×PBS buffer three times (5 min each wash) to remove any unbound exosomes, followed by flow cytometry analysis on an Accuri C6 flow cytometer (BD Biosciences, San Jose, Calif.) connected to an Intellicyt HyperCyt autosampler (IntelliCyt Corp., Albuquerque, N. Mex.) using Cy5 channel (649 nm). Data were processed and interpreted using FlowJo® software (Flowjo LLC, Ashland, Oreg.).

Statistical Analysis: Data are presented as the average±SEM (n=3 independent experiments). One-way analysis of variance (ANOVA) was used for data analysis to determine any statistically significant differences between two and multiple groups with Tukey's post-hoc analysis where appropriate using GraphPad Prism (v8.0) software. P 0.05 was considered significant.

### Duplex DNA Tethering of Exosomes (Exo-dsDNA):

Optimization of complementary DNA (DNA) concentration 12 μg of Exo-ssDNA were prepared with 20 μM Chol-DNA concentration, followed by incubation with different ratios of Cy5-conjugated complementary DNA (DNA′-Cy5) during annealing. Samples were sequentially incubated at 37° C. and RT for 15 min and 30 min respectively to accomplish the annealing procedure. Samples were washed with 1×PBS using 100 k MWCO filters to remove any excess reporter DNA. For the control experiments, native exosomes were incubated with DNA′-Cy5 for any non-specific labeling. The annealing efficiency of DNA′-Cy5 to Chol-DNA on Exo-ssDNA was analyzed by flow cytometry as described above using the Cy5 channel.

Optimization of annealing conditions: Solutions of 12 μg of Exo-ssDNA (20 μM Chol-DNA concentration) were prepared and were then incubated with 2× concentration of DNA′-Cy5 for annealing. In order to anneal the samples they were sequentially incubated at 37° C., 0° C. and RT for 15 min, 10 min and 30 min respectively. Samples were washed with 1×PBS buffer using 100 k MWCO filters to remove any excess DNA′-Cy5. Additionally, Exo-ssDNA-Cy5 samples, prepared using Chol-DNA-Cy5, were incubated under annealing conditions as control samples. The annealing efficiency of DNA′-Cy5 to Chol-DNA on Exo-ssDNA was analyzed by flow cytometry as described above using the Cy5 channel.

Preannealing Studies: Chol-DNA with 2× excess of DNA′-Cy5 was annealed by sequential incubation at 37° C., 0° C. and RT for 15 min, 10 min and 30 min respectively. 12 μg of exosomes were gently vortexed with a pre-annealed solution for tethering, 20 μM dsDNA tether concentration. Samples were washed with 100 k MWCO filters, followed by flow cytometry analysis as described above.

### DNA Tether Stability Studies:

Exo-ssDNA Stability: 120 μg of Exo-ssDNA-Cy5 (20 μM ssDNA tether concentration) were prepared using Chol-DNA-Cy5. Triplicate samples were incubated at 4° C. in 1×PBS buffer and 37° C. in simulated body fluid (10% FBS, 0.1% NaN₃, 100 mM HEPES in DMEM) for 24 h, 48 h, 72 h, and 1 week. At each time point, samples were incubated with anti-CD63 beads, rinsed three times (5 min each wash), followed by flow cytometry studies as described above using the Cy5-channel.

DNAse-1 stability: To assess the reversibility of DNA tethering on exosome membrane, both Exo-ssDNA-Cy5 and Exo-dsDNA-Cy5 (20 μM DNA tether concentration) were incubated with 2.5 units of DNase-I (New England Biolabs, Ipswich, Mass.) suspended in 1× DNase I Reaction Buffer (New England Biolabs, Ipswich, Mass.) for 15 min at 37° C. Post incubation, beads were magnetically separated and thoroughly rinsed in 1×PBS prior to flow cytometric analysis as described above.

AS1411-conjugated Exosomes: DNA′-AS1411 with complementary region to Chol-DNA (5′-GGTGGTGGTGGTTGTGGTGGTGGTGGTTAGCTATGGGATCCAACTGCAGT-3′ (SEQ ID NO: 13)) was pre-annealed to Chol-DNA using standard annealing conditions. The pre-annealed Chol-dsDNA-As1411 was vortexed with exosomes to prepare Exo-dsDNA-AS1411, 20 μM dsDNA tether concentration.

Internalization Studies: Native exosomes, Exo-ssDNA-Cy5 and Exo-dsDNA-Cy5 were labeled with PKH26 and PKH67 dyes (Sigma-Aldrich, St. Louis, Mo.) according to manufacturer's instruction. The EVs were precipitated by centrifugation (100,000×g for 3 hours) and resuspended in the diluent C. The respective dye was diluted (4:1000) in diluent C and added to the EVs followed by rigorous mixing for 90 seconds. The excess dye was quenched with 1% BSA in Diluent C for 5 min at RT and filtered using 300 K Da. M.W.C.O filter (Sartorius, Germany). Labeled exosomes were resuspended in 1×PBS and used for in vitro studies.

HEK293 and MIAPaCa-2 cells were seeded at 2.5×10³ cells/cm² on collagen type-I coated coverslips (Electron Microscopy Services, Hatfield, Pa.) and allowed to adhere for 4 hours prior to the addition of labelled exosomes. Exosomes were added to a final concentration of 20 μg/ml for designated time points. Post incubation, the plasma membrane bound EVs were washed-off using stripping buffer (pH 2.5; 14.6 g NaCl, 2.5 ml acetic acid, 500 ml distilled water) for 1 min and cell were fixed in 3.33% paraformaldehyde (PFA; Electron Microscopy Services, Hatfield, Pa.) at room temperature (RT) for 15 min. Excess PFA was quenched with 1% bovine serum albumin (BSA; Sigma-Aldrich, St. Louis, Mo.) in PBS and the monolayer was rinsed thoroughly four times with 1×PBS. Cells were permeabilized with 0.1% Triton-X (Sigma-Aldrich, St. Louis, Mo.) for 1 min and stained for 10 min at RT with Alexafluor® 647-phalloidin/Alexafluor® 488-phalloidin (Invitrogen, Carlsbad, Calif.) diluted to 3:80 in 1×PBS. Nuclei was stained with 1:1000 (in 1×PBS) Hoechst 33342 (ThermoFisher Scientific, Waltham, Mass.) for 5 min, rinsed thoroughly and mounted using prolong gold (Invitrogen, Carlsbad, Calif.). Imaging was performed using Zeiss LSM 880 confocal microscope (Carl Zeiss Microscopy GmbH, Jena, Germany) under constant settings across all the different treatment groups and analyzed using Imaris microscopy analysis software (Bitplane AG, Zurich, Switzerland). The amount of exosome internalized was evaluated by comparing the relative fluorescence intensities measured in NIH imageJ software post background subtraction. Five random pictures were captured per treatment group for the mean fluorescence measurement evaluations.

### Bioprinting Exosomes for Solid-Phase Presentation:

A solution of 10 micrograms per milliliter (μg/ml) of Exo-FasL exosome containing a final concentration of 10% glycerol was used as the bioink. 50 overprints (OPs) of exosome bioink was printed on collagen type-1 coated coverslips to create patterns of 1.25 mm×1.75 mm corresponding to a total concentration of 76 ng of total exosome protein. Post overnight rinsing in PBS to wash-off unbound exosomes, PCI13 cells were seeded at a density of 2.5×10³ cells/cm². Post 24 hours, cells were stained with live/dead cell viability assay for mammalian cells (ThermoFisher Scientific, Waltham, Mass.) according to manufacturer's instruction. This kit utilizes Calcien AM and ethidium bromide to differentiate between live and dead cells. Post staining, imaging was performed on ZEISS LSM 880 confocal microscope (Carl Zeiss Microscopy GmbH, Jena, Germany). Quantification of mean fluorescence intensities post background subtraction was performed on NIH Image J software by selecting a region of region corresponding to the printed exosome pattern.

### In Vivo Studies:

Animals: C57BL/6 and C57BL/6-Tg (Foxp3-DTR-eGFP; referred to as C57BL/6-DTR here) and BALB/c mice were purchased from Jackson Laboratory (Bar Harbor, Me., USA), F1 mice were produced by crossing C57BL/6-DTR and BALB/c, and all mice were maintained under standard conditions in the Institute for Cellular Therapeutics barrier facility. The animals were cared for according to the University of Louisville and National Institutes of Health animal care and use guidelines. Female, 6- to 8-week-old C57BL/6(H-2b) and F1(C57BL/6-DTR×BALB/c, H-2b×d) mice were used as splenocyte donors and recipients, respectively.

Preparation of CFSE-Labeled Splenocytes: Spleens were collected from native C57BL/6 female mice, processed into single-cell suspension, and red blood cells were lysed using ACK (ammonium chloride-potassium lysis buffer) solution. Splenocytes were passed through sterile nylon mesh strainers with 100 μm pores, centrifuged, and washed several times with PBS (Gibco, Gaithersburgh, Md., USA). Cells were incubated with 2.5 micromolar (μM) CFSE in PBS for 7 min at room temperature, and labeling reaction was stopped by the addition of an equal volume of FBS (fetal bovine serum, RMBIO). CFSE-labeled cells were then washed twice with PBS, and each female F1 mouse was injected through the tail vein with 5×10⁶ cells in 600 μL of PBS.

Treatment and in Vivo Tracking of Donor Cells in F1 Recipients: F1 mice were divided into four groups and subjected to two intraperitoneal (i.p.) treatments with 40 μg of exosomes engineered with SA-FasL (ExossDNA-SA-FasL) at 2 and 24 h after CFSE-labeled splenocytes injection. An equal amount of Exo-ssDNA-biotin, the same dose of soluble SA-FasL used for exosome engineering, and saline (PBS) were used as controls. Cells from mesenteric lymph nodes and spleens of treatment and control groups were harvested at 72 h post cell injection, erythrocytes were lysed with ACK lysis buffer, and cells were washed with PBS. Cells were incubated for 15 min at room temperature with anti-mouse CD16/CD32 (Mouse FC block, BioLegend, San Diego, Calif., USA) antibody to block Fc receptors. Samples were then stained with antibodies to mouse CD3-V500, CD4-Alexa Flour 700, CD8-APC Cy7 (BD Biosciences), and MHC class I (H2Kd)-APC (BioLegend) molecules for 25 min at 4° C. and washed with PBS prior to analysis. The cells were run on the LSR II (BD Biosciences) flow cytometry, and the data were analyzed by BD FACSDiva software and graphed using GraphPad Prism. Representative gating of splenocytes is shown in FIG. 2.

Functionalization of Exosomes with an Antibody:

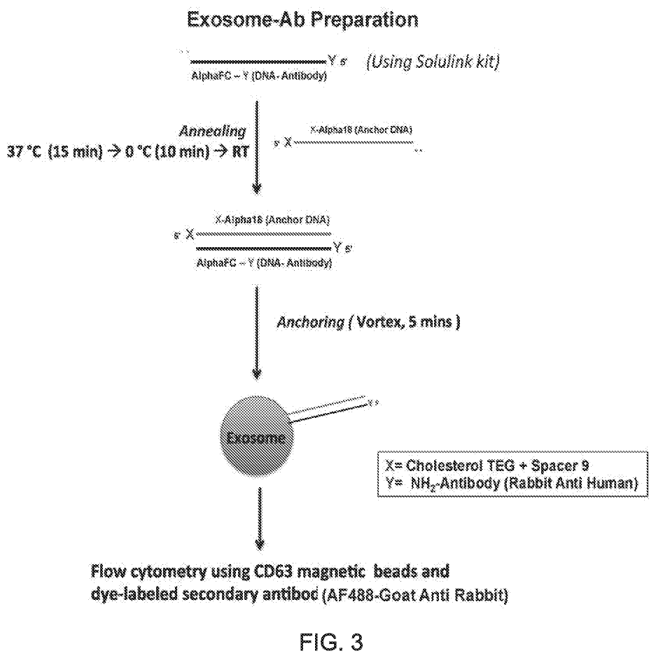

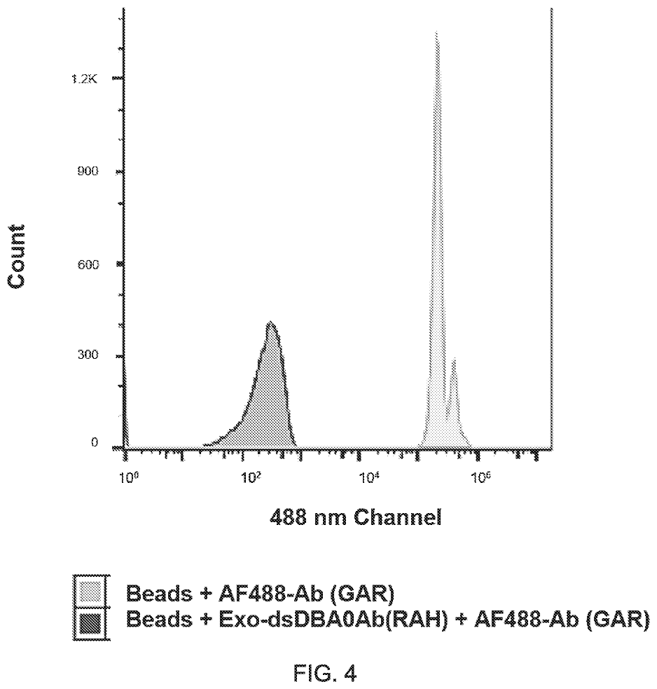

The procedure employed is summarized in FIG. 3. Antibody-DNA conjugation was performed using the Solulink protein-oligo conjugation kit (catalog S-9011-1, Solulink), which uses bisaryl hydrazone conjugation chemistry. 5′-aminated DNA (NH₂—C12-αFC; NH₂—C₁₂-TT ATGGGATCCAACTGCAGT (SEQ ID NO: 14)) was ordered from Integrated DNA Technologies (IDT) and was functionalized with 4-FB reagent using 5:1 (4-FB: DNA) molar ratio. 130 nmoles of purified 4-FB-αFC DNA with molar substitution ratio of 0.77, was obtained after the functionalization. Rabbit Anti-Human Antibody (RAH) was functionalized with S-HyNic reagent separately and was purified using MicroSpin columns. The modified antibody and the DNA were dissolved in conjugation buffer (100 mM phosphate, 150 mM NaCl, pH 6) and were allowed to react for 2 hours at room temperature. Unreacted DNA was removed using 100 kDa filters MWCO centrifugal filters. The conjugation of antibody-DNA was verified spectrophotometrically, by the presence of the bisaryl hydrazone linkage (λmax=354 nm) formed between S-HyNic and 4-FB. The procedure is summarized on FIG. 3 and was carried out by annealing Chol-DNA and Antibody-DNA′ by sequential incubation at 37° C., 0° C. and RT for 15 min, 10 min and 30 min respectively. 20 μg of exosomes were gently vortexed with pre-annealed solution for tethering, final DNA tether concentration was 2 μM. Samples were washed with 100 k MWCO filters, followed by reverse spin to get Rabbit Anti-Human antibody-functionalized exosomes (Exo-dsDNA-Ab (RAH)). Analysis was performed using flow cytometry and by incubating the beads with AF488-labeled Goat Anti-Rabbit antibody. A clear shift of fluorescence intensity in 488 nm channel verified the successful conjugation (FIG. 4).

### Preparation of Exosome-dsDNA-Biotin:

Chol-DNA and DNA′-Biotin were pre-annealed as described above. 20 μg of exosomes were gently vortexed with preannealed solution for tethering (20 μM dsDNA tether concentration). Samples were washed with 100 k MWCO filters, followed by reverse spin to get Exo-dsDNA-Biotin.

Exosome with 10 μM biotinylated DNA as described above. 20 μg exosomes were incubated with 100 ng SA-FasL for 30 min at 37° C. Post incubation, FasL-exosomes were isolated by the miniSEC method described in the exosome isolation protocol. Jurkat cells (20⁶/mL) were cultured in freshly prepared RPMI-1640 medium supplemented with 10% ED-HI-FBS for 48 hours. 20 μg exosomes anchored with 100 ng SA-FasL were added to the media and incubated for 12 hours. Native exosomes and biotin-DNA-exosomes were used as controls. Apoptosis of Jurkat cells was measured by flow cytometry using an Annexin V assay (Beckman Coulter, Brea, Calif.).

### Click Chemistry on Exosomes:

Preparation of Exosome-dsDNA-N₃: 10 nmole of Chol-DNA were incubated with 10 nmole of N₃-modified complementary DNA strand (DNA′-N₃) in PBS buffer for annealing (37° C. (15 min)→0° C. (10 min)→RT (30 min)). Preannealed Chol-dsDNA-N₃ was vortexed with 100 μg of exosomes in 500 μL PBS buffer to prepare Exo-dsDNA-N₃ (20 μM azide concentration). The sample was concentrated to 200 μM azide concentration using ultra centrifugal filters (MWCO=100 k) for the click reaction.

Click reaction with SF-488 dye: 25 μL of Exo-dsDNA-N₃ (200 μM azide concentration) was incubated with 25 μL of SF488-DBCO (1 mM stock) and 5 μL DMSO at 4° C. for 16 hours. The sample was washed using 100 k MWCO filters to remove unbound SF488-DBCO, followed by flow cytometry analysis using 488 nm channel. For the control experiment, 50 μg of native exosomes, were incubated with SF488-DBCO under exact same conditions.

Click reaction with PEG30 k: 25 μL of Exo-dsDNA-N₃ (200 μM azide concentration) was incubated with 25 μL of PEG30 k-DBCO (1 mM stock) and 5 μL DMSO at 4° C. for 16 hours. The sample was washed using 100 k MWCO filters to remove unbound PEG30 k-DBCO, followed by analysis using DLS. For the control experiment, 50 μg of native exosomes, were incubated with PEG30 k-DBCO under exactly the same conditions.

Cu-click with Cy5-alkyne: 50 μL of Exo-dsDNA-N₃ (50 μg, 20 μM DNA tether concentration), 15 μL of Cy5-alkyne (1 mM stock in DMSO), 2.5 uL of sodium ascorbate (100 mM stock) were mixed in 12.5 μL of 1×PBS. The solution was degassed several times by blowing with argon, to remove any dissolved oxygen. 20 μL of degassed solution of 100 mM CuSO₄/THPTA (1:5) was added to initiate the reaction and was allowed to run for 3 hours at room temperature with gentle shaking. The product (Exo-dsDNA-Cy5) was purified with 100 k MWCO, followed by flow cytometry studies as described above using Cy5 channel. For the control experiment, 50 μg of native exosomes, were incubated with Cy5-alkyne, CuSO₄, sodium ascorbate under exact same conditions.

### Results and Discussion

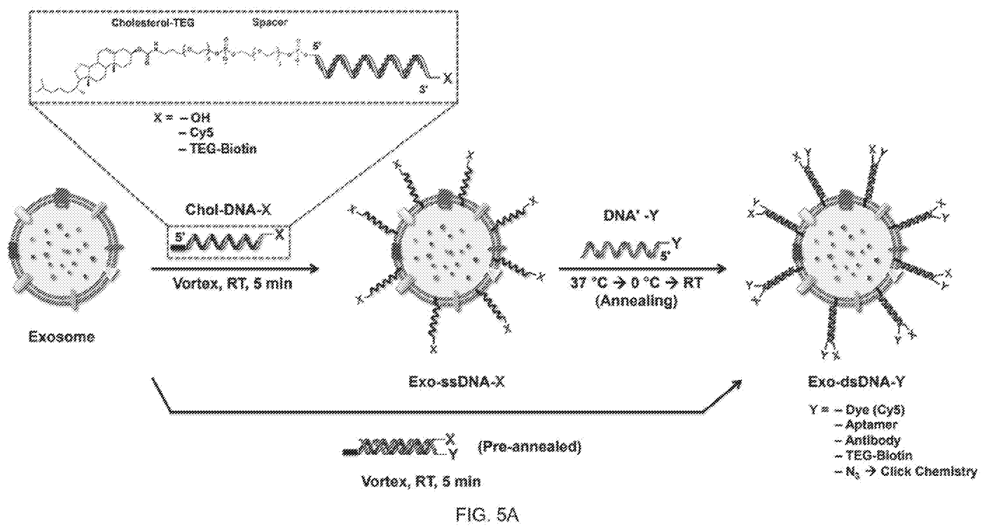

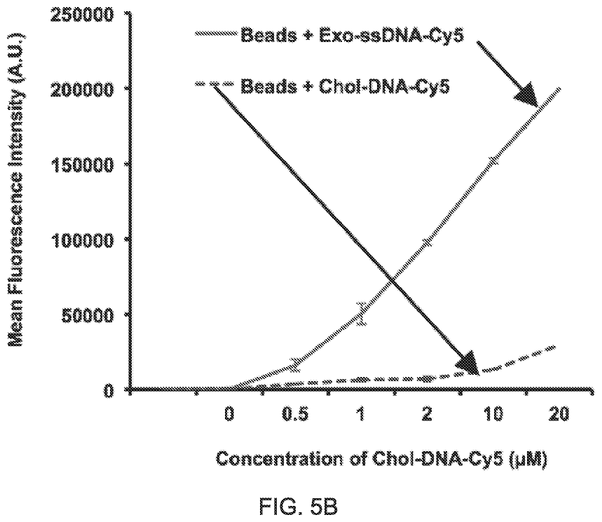

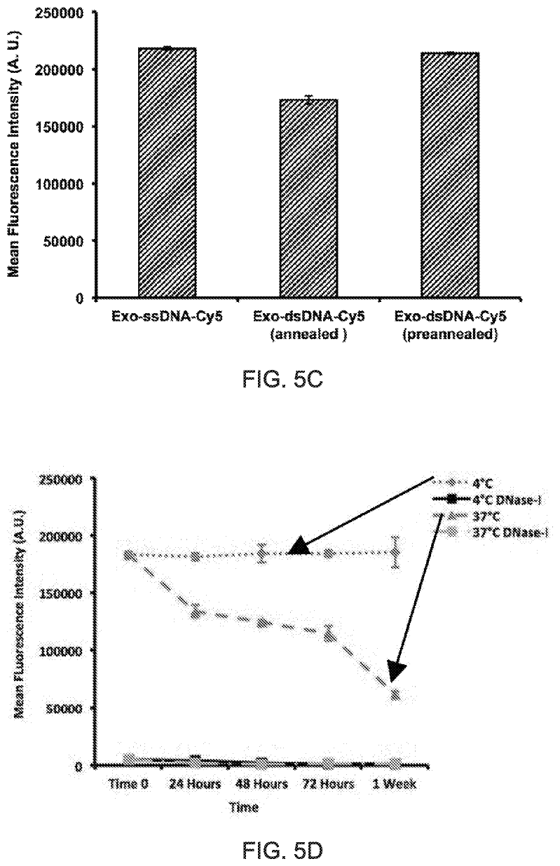

DNA tethers for exosomes: In order to investigate the tethering efficiency of cholesterol modified oligonucleotides onto an exosome membrane, in a non-limiting exemplification of the procedure, an 18-mer DNA tether (5′-ACT GCA GTT GGA TCC CAT-3′ (SEQ ID NO: 15)) with cholesterol modification on the 5′ end (Chol-DNA) was synthesized by simply vortexing the exosomes with Chol-DNA in buffered solution at ambient room temperature, FIG. 5A. An additional 5′-tetra(ethylene glycol) (“TEG”) spacer, spacer 9, was inserted between the cholesterol moiety and DNA to reduce steric and electrostatic repulsion of the 18-mer DNA with the negatively charged exosome membrane. Results are presented in Table 1 and show a good relationship between the expected and measured molar mass after the 3′-chain end of the DMA was modified by an exemplary small functional molecule, Cy5 dye, or by a biological molecule, exemplified by Biotin. Spacer 9 can be between 1 and 5 TEG units long. The spacer can actually comprise other biocompatible polymers with similar molecular weight.

TABLE 1
Results of chain end modification of 18-mer DNA

| | 5′- | 3′- | Sequence | Expected | Observed |
|---|---|---|---|---|---|
| Name | modification | modification | (5′ TO 3′) | Mass | Mass |
| Chol- | Cholesterol | — | ACTGCAGTTGGATCCCAT | 6466.9 | 6466.4 |
| DNA | TEG, Spacer 9 | | (SEQ ID NO: 15) | | |
| Chol- | Cholesterol | Cy5 | ACTGCAGTTGGATCCCAT | 6901 | 6900 |
| DNA- | TEG, Spacer 9 | | (SEQ ID NO: 15) | | |
| Cy5 | | | | | |
| Chol- | Cholesterol | Biotin TEG | ACTGCAGTTGGATCCCAT | 6936.58 | 6976 |
| DNA- | TEG, Spacer 9 | | (SEQ ID NO: 15) | | |
| Biotin | | | | | |
| DNA'- | Cy5 dye, Spacer | — | ATGGGATCCAACTGCAGT | 6416.47 | 6420 |
| Cy5 | 9 | | (SEQ ID NO: 16) | | |

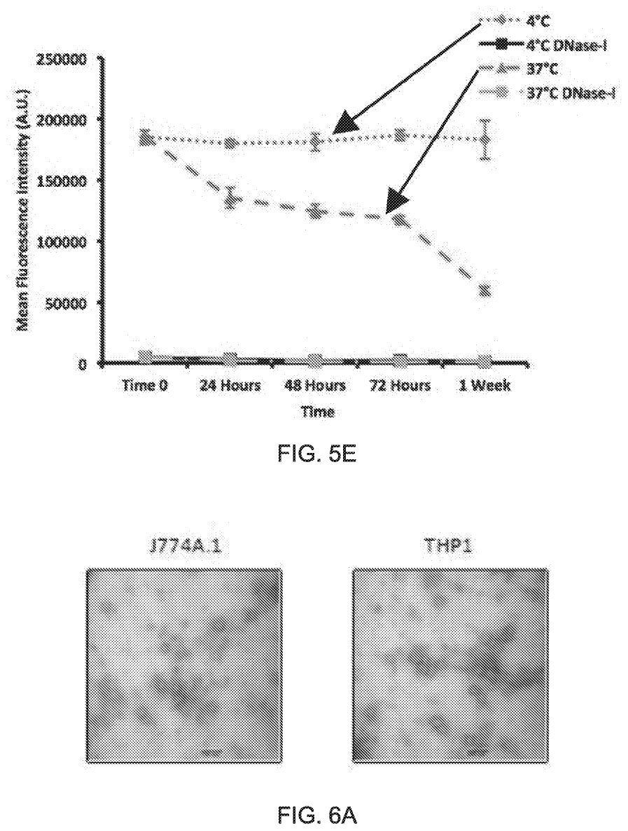

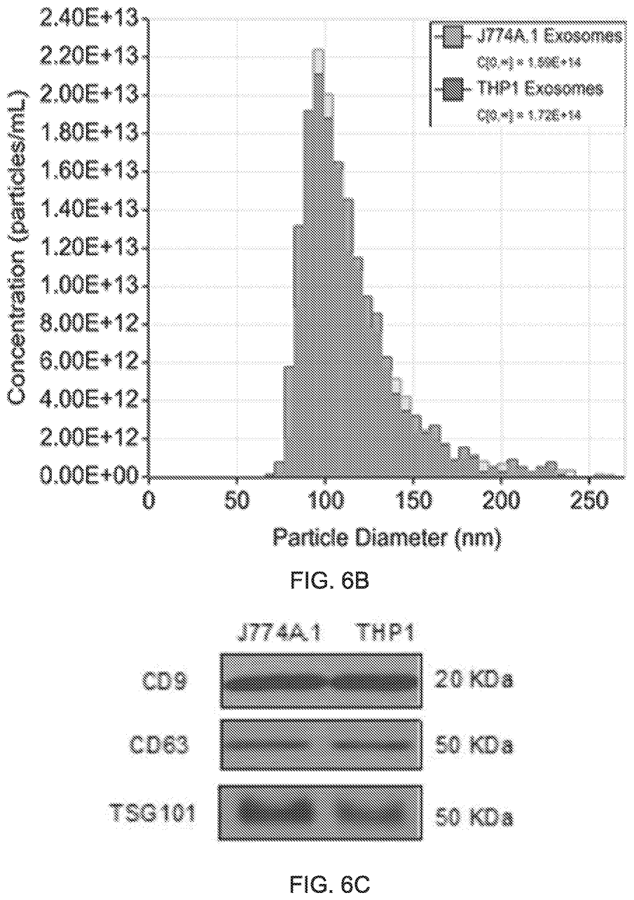

Exosomes, isolated from THP1 cells, were anchored using different concentrations of Chol-DNA (FIGS. 6A-6C). DLS studies showed approximately 5 nm increase in the exosome diameter at all DNA tether concentrations. However, the Zeta potential of native exosomes (−10 mV) decreased with increasing concentration of the tethered DNA (−20 mV for 20 μM DNA tether concentration), suggesting successful tethering of different numbers of DNA on the external surface of the membrane.

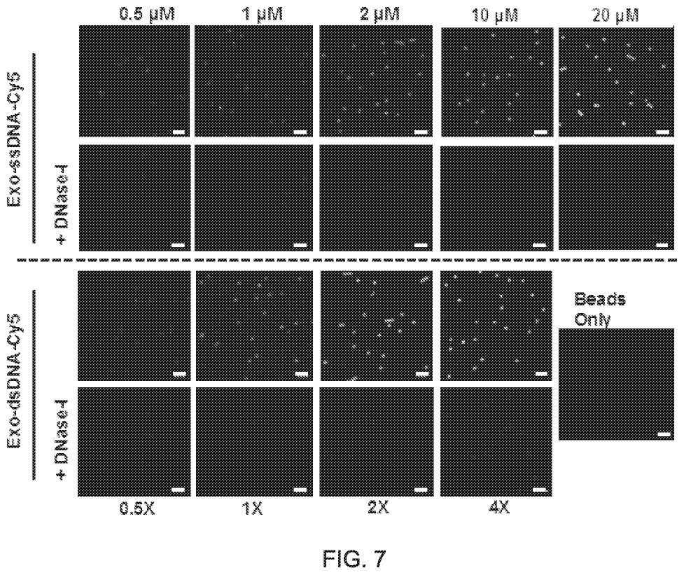

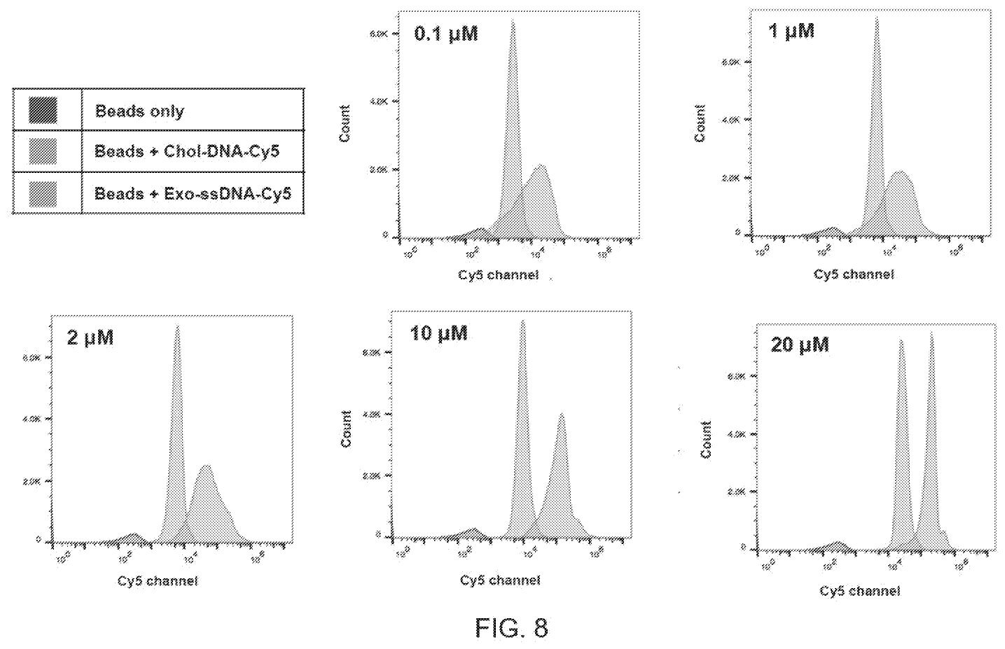

FIG. 5A provides a schematic of the process employed to prepare the membrane modified exosome. FIGS. 1, 5B, 7, and 8 show the flow cytometry analysis using Cyanine 5 (Cy5) labeled DNA tether (Chol-ssDNA-Cy5) which displayed a linear increase in Cy5 signal with increasing Chol-DNA concentrations.

The selected oligonucleotide, which includes DNA, RNA, PNA or L-DNA, can comprise functionality at either the 5′-chain end of 3′-chain end or in nucleotide units close to the chain ends. Indeed the spacer, of one of the selected spacers, can also comprise functionality for further reactions, e.g., a photo-responsive unit.

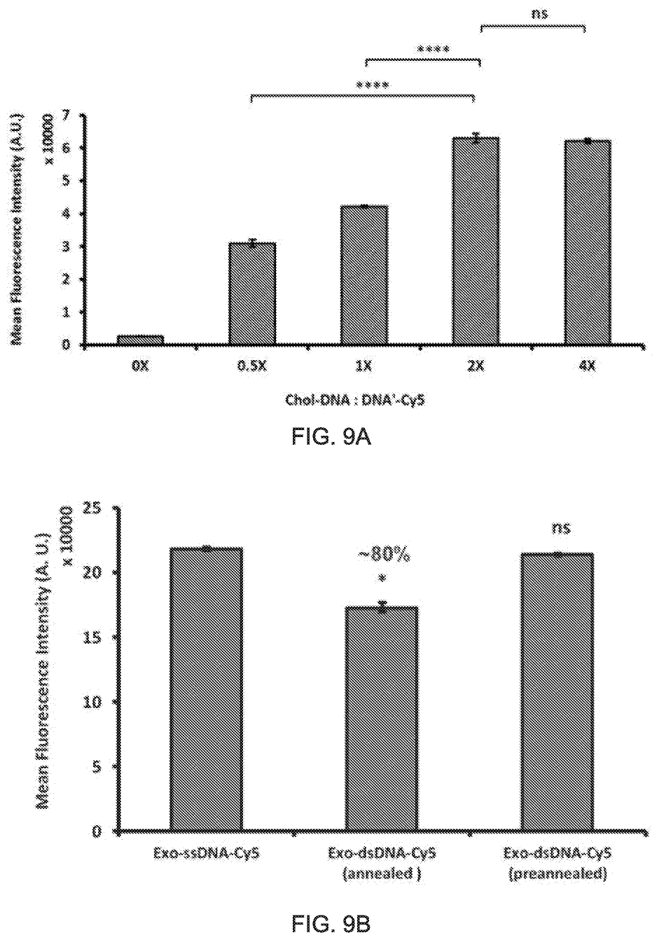

To investigate the availability of the exosome-tethered DNA for further functionalization a complementary Cy5-labeled DNA strand (5′-ATG GGA TCC AAC TGC AGT-3′; DNA′ (SEQ ID NO: 16)) was prepared and annealing studies showed that approximately 80% of the single-stranded DNA was exposed outside the lipid membrane for hybridization to the complementary strand (FIGS. 9A and 9B). This positive result is in stark contrast to prior art procedures where the objective was to load hydrophobic units inside the membrane. FIG. 5C shows that pre-annealing the Chol-DNA and DNA′ before tethering onto exosomes, showed same membrane linkage efficiency as single-stranded DNA tether.

The stability of the Chol-ssDNA tether on the exosome was assessed at 4° C. in PBS buffer and at 37° C. in simulated body fluid. Both Exo-ssDNA-Cy5 and Exo-dsDNA-Cy5 were incubated at respective conditions and were analyzed by flow cytometry at different time points. No decrease in the mean fluorescent intensity (MFI) was observed at 4° C. for both the systems, depicting the high stability of DNA anchoring under storage conditions, FIGS. 5D and 5E.

However, at 37° C., a linear drop was observed in the MFI, which is potentially due to the protein shedding of CD63 from the exosome membrane, hence affecting the binding to Anti-CD63 magnetic beads and thereby reducing the overall signal.

In addition to stability, a complete removal of the DNA tethers was observed after treatment with DNAse enzyme, highlighting the reversibility of the DNA modification of exosomes.

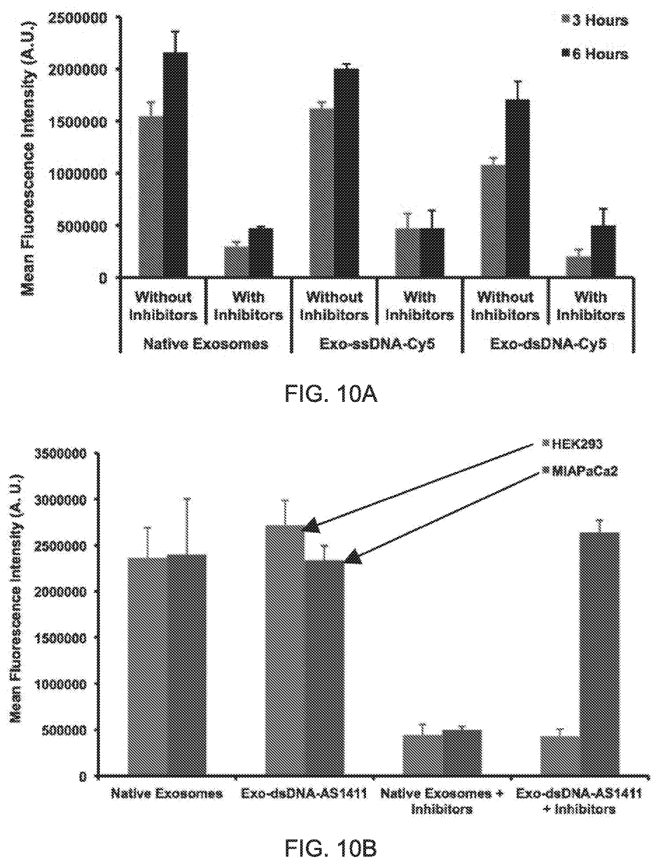

AS1411-conjugated Exosomes: In order to highlight the advantages of DNA functionalization of exosomes, the effect of AS1411 aptamer cellular uptake of exosomes was investigated. The AS1411 aptamer is an oligonucleotide sequence designed to bind nucleolin. This sequence can be directly displayed on exosomes using a “tail” that binds to the Chol-DNA strand. The studies were performed in two cell lines, human embryonic kidney cells (HEK293) and human pancreatic cancer cells (MiaPaCa2). MiaPaCa2 cells are known to express nucleolin protein on the cell membrane (Hovanessian et al., PLoS One, 2010, 5:e15787), which can allow AS1411-mediated internalization while HEK293 cells do not express nucleolin, and can serve as a negative control (Biomaterials, 2014, 35:3840-3850). Exosomes, prepared with and without AS1411, were incubated with the cells and imaged after 6 hours. In parallel, exosome samples were also tested in the presence of inhibitors (heparin and beta-methylcyclodextran), which inhibits the two major pathways for exosome internalization. It was observed that exosomes with AS1411 were able to internalize in the nucleolin-expressing cells, even in the presence of inhibitors, while low internalization efficiency was observed in HEK293 cells, FIG. 10B. On the other hand, native exosomes showed very low internalization in the presence of inhibitors, while no difference was observed between the two cell lines in the absence of inhibitors. These results underscore the effect of the presence of the AS1411 aptamer and show that exosome internalization pathways can be easily altered using this approach.

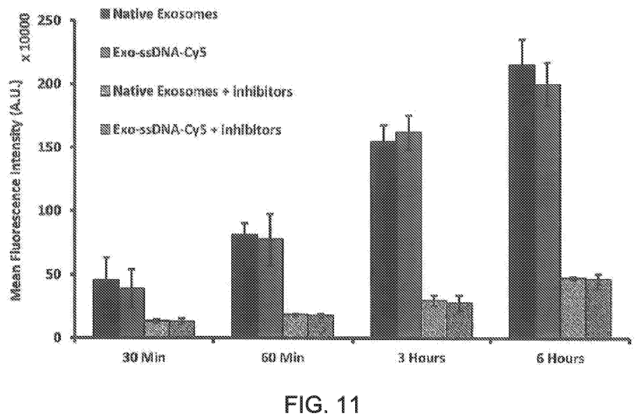

Internalization Studies: To investigate whether the presence of negatively charged DNA on the exterior of the membrane of the exosomes affects the internalization efficiency internalization studies were performed with Human Embryonic Kidney (HEK293) cells. HEK293 cells were incubated with native exosomes as well as Exo-ssDNA-Cy5 and Exo-dsDNA-Cy5. Internalization was also tested in the presence of two inhibitors, heparin and beta-methylcyclodextran, which inhibit the internalization through heparin sulphate proteoglycans and lipid-raft mediated internalization, respectively (Sercombe et al. “Advances and challenges of liposome assisted drug delivery”, Frontiers in pharmacology, 2015, 6:286). A similar internalization rate was observed for native exosomes, Exo-ssDNA-Cy5 and Exo-dsDNA-Cy5, FIG. 10A, although Exo-dsDNA-Cy5 had a slightly lower rate, possibly due to a more negative surface charge. Time-dependent internalization of native exosomes, Exo-ssDNA-Cy5 and Exo-dsDNA-Cy5 is shown in FIG. 11. A significant drop in the cell internalization was observed in the presence of inhibitors. However, complete inhibition was not observed in the presence of inhibitors, due to other potential modes of exosomes internalization inside the cells, FIG. 10B.

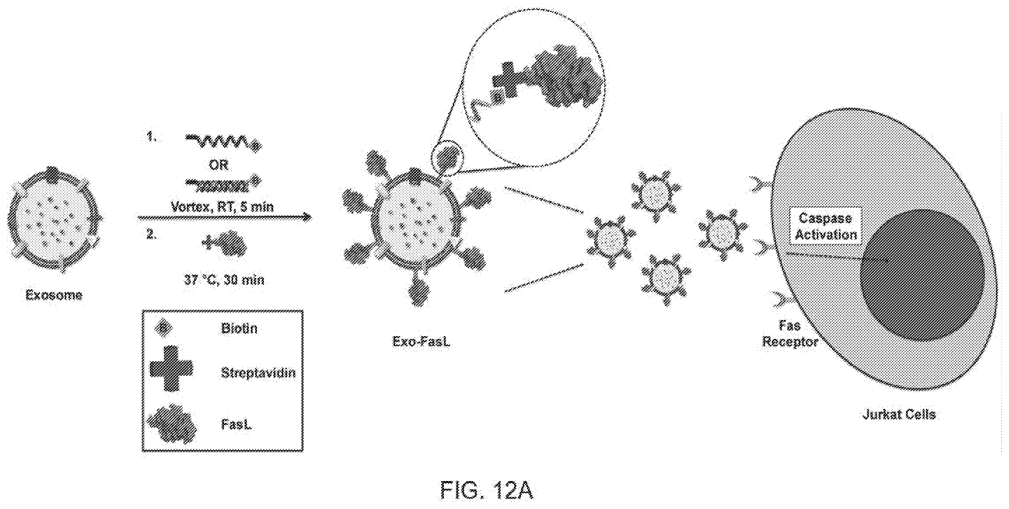

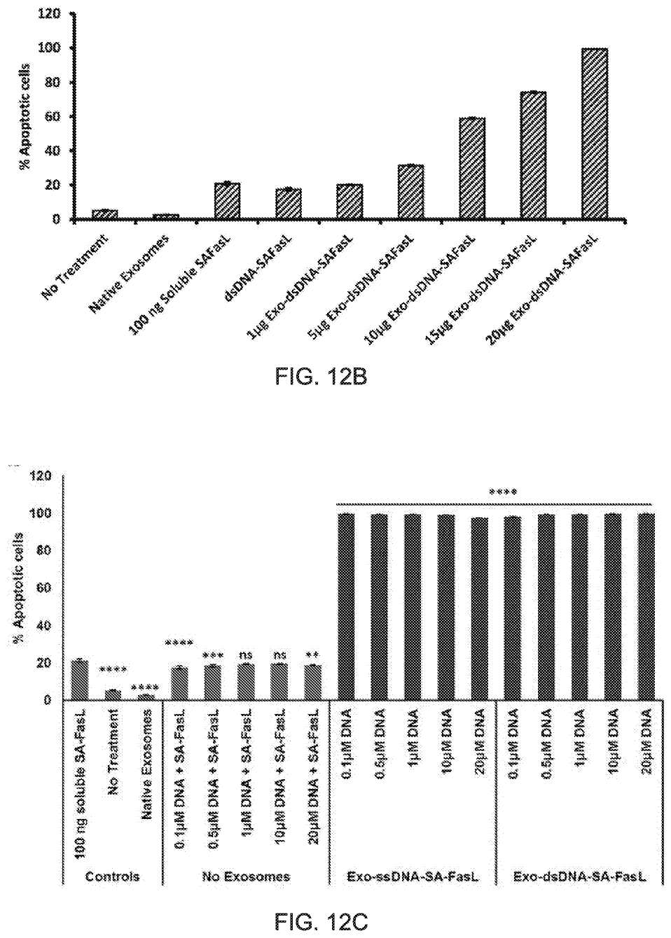

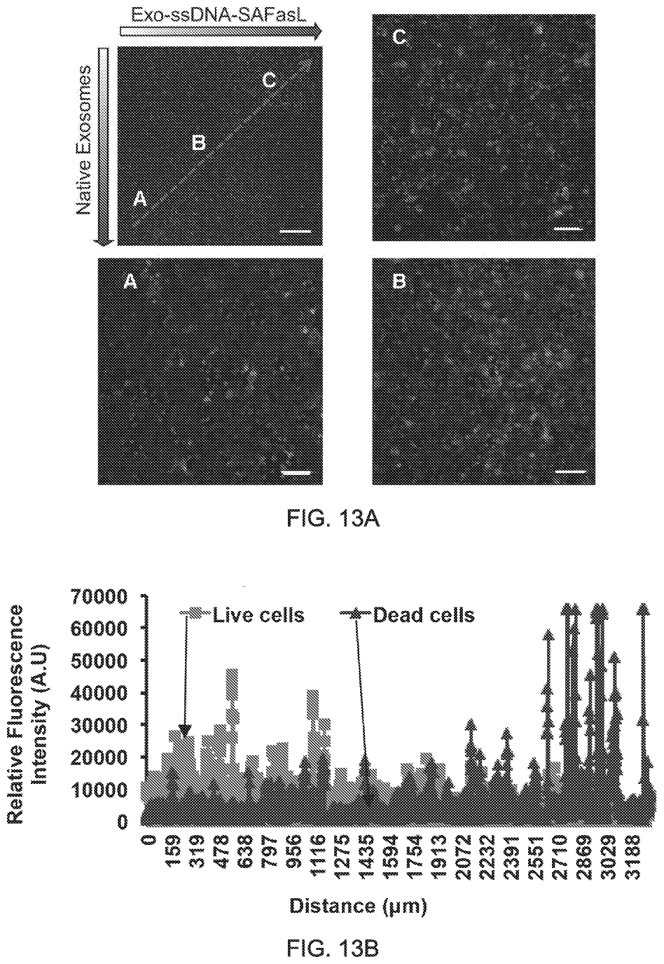

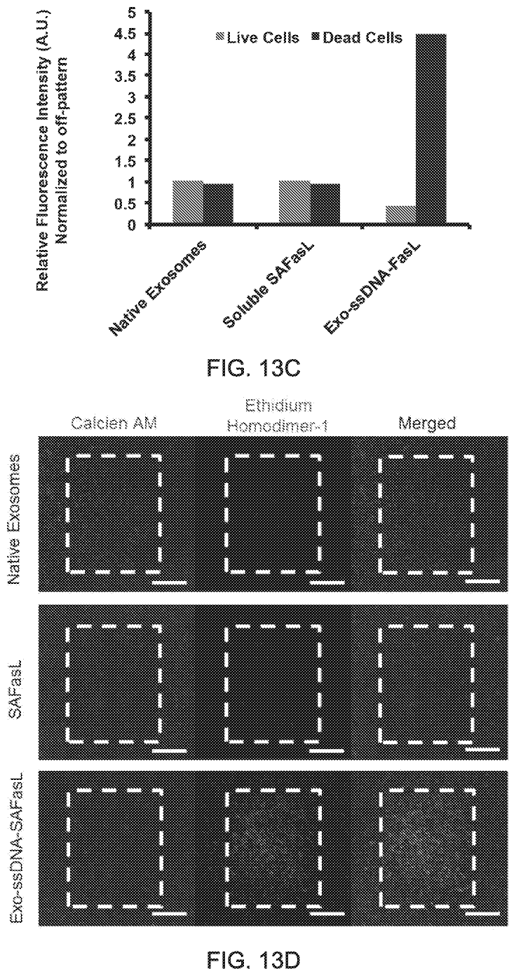

Apotosis Assay: Immunomodulatory agents such as FasL and PDL-1 on tumor exosomes (TEX) have been reported as a contributor to the spontaneous T-cell apoptosis in numerous studies (Hong et al.; Theodoraki et al. “Clinical significance of PD-L1+ exosomes in plasma of Head and Neck Cancer patients”, Clinical Cancer Research 2018, 24(4):896-905). This inspired examination of THP1 membrane modified exosomes, which were prepared by engineered conjugation of streptavidin-FasL (Exo-ssDNA-FasL) onto their membrane surfaces, and their biological activity was evaluated using Jurkat cells, FIG. 12A. A dose-dependent interaction resulted in a corresponding dose-dependent increase in Jurkat cell apoptosis as evaluated by Annexin V—PI staining, FIGS. 12B and 12C. The lowest concentration i.e., 1 μg exosome protein resulted in 17.46% (±0.86%) apoptosis whereas 20 μg exosome protein resulted in 99.3% (±0.03%) apotoxic cells. On the other hand, native THP1 exosomes on their own and 100 ng of SAFasL did not induce significant apoptosis. Similar apoptosis was also observed with Exo-dsDNA-FasL, FIGS. 13A-13D. This data validates that response modifiers that are biologically active can be conjugated to the surface of an exosome using the disclosed membrane DNA-tethering technology.

### Bioprinting Exosomes for Solid-Phase Presentation:

Bioprinted exosomes induce spatially controlled apoptosis in cancer cells: Although FasL is considered to show promise for cancer therapy, major side effects have precluded its systemic use. One way to mitigate off-target negative effects is to locally deliver FasL immobilized onto scaffold materials. To evaluate the feasibility of this procedure a bioprinting technology that could spatially control exosome-microenvironments and therefore locally modify cell behavior, thereby limiting off-target responses, was examined. As a proof-of-concept, Exo-ssDNA-Cy5 was printed onto collagen type-1 coated coverslips to create persistent patterns, as shown in FIGS. 13A-13C: the ink concentration=100 μg/ml and droplet volume=63.56±4.83 μL with a droplet velocity=2.045±0.28 m/s. The images show that tethering of DNA onto exosomes did not hamper the ability of membrane-associated integrins to interact with collagen binding domains.

To assess whether the bioprinted oligonucleotide-tethered exosomes are biologically active, Exo-ssDNA-SAFasL were printed and subjected to post overnight rinsing, then PCI13 cells were seeded onto the coverslips. The printed Exo-ssDNA-SAFasL pattern resulted in spatially restricted apoptosis in PCI13 cells (FIGS. 13B and 13D). There was no significant apoptosis on native exosome patterns and on off-pattern regions, suggesting that Exo-ssDNA-SAFasL are biologically active when present in the solid-phase. FIG. 13A shows a combinatorial array of Exo-FasL and native exosomes that were printed with increasing concentration of FasL along the diagonal, top right image. Quantification of live and dead cells along the diagonal showing increasing apoptosis rate with increasing concentration of FasL (FIG. 13B). The bar plot in FIG. 13C shows the effect of native exosomes, free SAFasL and Exo-FasL on cell death in a study comparing native exosomes, free SA-FasL and exo-FasL on apotosis of PCL13 cells. There was a significant fluorescence from dead cells in the presence of deposited FasLanchored exosomes with minimum dead cells observed when native exosomes or DNA modified exosomes were deposited on the coverslips.

In one embodiment of this invention exosome membranes can be engineered using hydrophobically modified oligonucleotides. Importantly, tethering of oligonucleotides to the exterior of the membrane does not result in any changes to either the native exosome membrane protein accessibility, or cellular uptake physiology. This finding prompted further exploration of the possibility tethering biochemically active cargo, such as AS1411 or SAFasL to engineer the cell-exosome interaction biology and application of this engineering approach to modulate in vivo immune responses were also demonstrated.

When compared to the polymeric and liposomal-based nanoparticle delivery approaches used for treating a broad range of pathologies over the last 40 years utilizing EVs (Torchilin, “Recent advances with liposomes as pharmaceutical carriers”, Nature reviews, Drug discovery, 2005, 4(2):145; Allen et al. “Liposomal drug delivery systems: from concept to clinical applications”, Advanced drug delivery reviews, 2013, 65(1):36-48; Sercombe et al. “Advances and challenges of liposome assisted drug delivery”, Frontiers in pharmacology, 2015, 6:286; Zylberberg et al. “Pharmaceutical liposomal drug delivery: a review of new delivery systems and a look at the regulatory landscape”, Drug delivery, 2016, 23(9):3319-29), the specifically modified exosomes loaded with exogenous cargos, hold promise as ideal delivery vehicles because they are naturally occurring nanoparticulates, evolved explicitly for intercellular communication, transporting a wide array of cargo throughout the body. While the detailed mechanisms of exosome signaling and delivery of molecular cargo remains to be elucidated (Mulcahy et al. “Routes and mechanisms of extracellular vesicle uptake”, Journal of extracellular vesicles, 2014, 3(1):24641; French et al. “Extracellular vesicle docking at the cellular port: extracellular vesicle binding and uptake”, Seminars in cell & developmental biology, Academic Press, 2017, 67:48-55), the present data indicates that DNA-tethered EVs represent a unique reversible way to engineer designer exosomes with versatile functionalities.

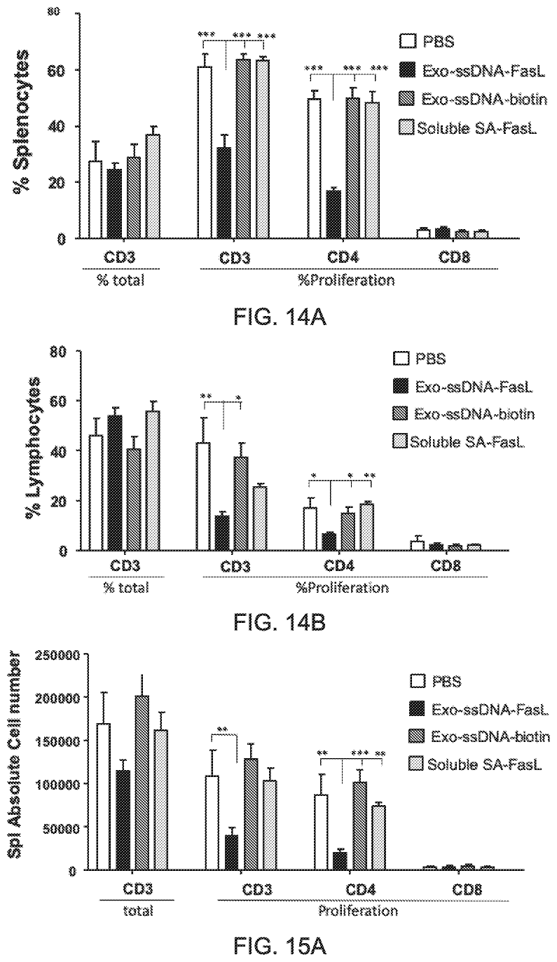

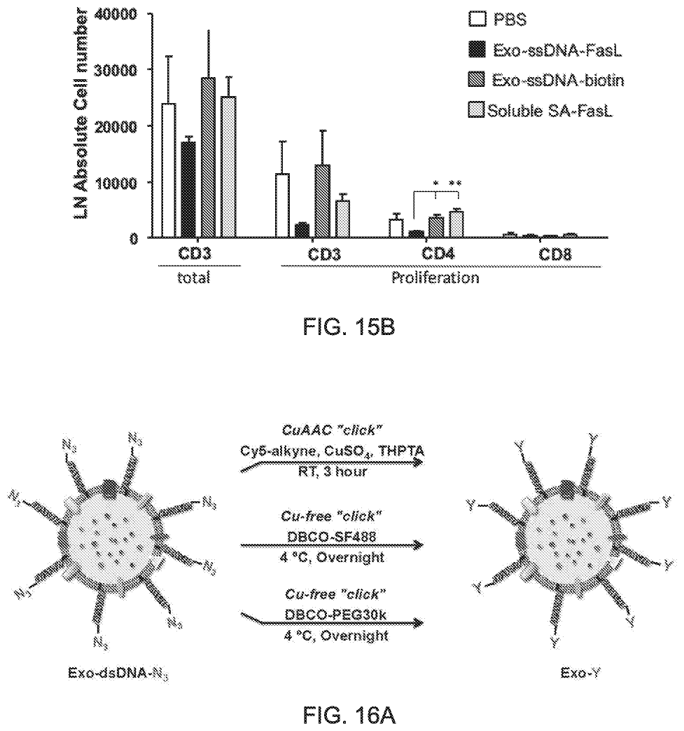

In vivo studies: To provide evidence that exosomes engineered with biologics are viable drug candidates, we targeted the Fas death pathway as a model system since it plays a critical role in T cell homeostasis. We have previously shown that SA-FasL can be positionally and transiently displayed on the surface of cells or tissues (Yolcu et al. “Induction of Tolerance to Cardiac Allografts Using Donor Splenocytes Engineered to Display on Their Surface an Exogenous Fas Ligand Protein”, J. Immunol, 2008, 181: 931-939; Yolcu et al. “Pancreatic Islets Engineered with SA-FasL Protein Establish Robust Localized Tolerance by Inducing Regulatory T Cells in Mice”, J. Immunol, 2011, 187:5901-5909). Transplantation of the engineered cells and tissues into allogeneic recipients induces tolerance via apoptosis in responding alloreactive T cells through engagement of Fas receptor (Yolcu et al., J. Immunol, 2011). An in vivo MLR assay was used in a parent-to-F1 mouse model to assess activity of the Exo-ssDNA-SA-FasL on donor T cell proliferation. The spleen cells from donor mice (C57BL/6) were labeled with carboxyfluorescein succinimidyl ester (CFSE) and adoptively transferred into F1 (C57BL/6-DTR×Balb/c) recipients followed by two separate administrations of Exo-ssDNA-SAFasL via systemic intraperitoneal (i.p.) injections at 2 and 24 h postcell infusion. We observed a significant reduction in the percentage of proliferating donor CD3+ and CD4+ T cells in the spleen and lymph nodes of F1 mice treated with ExossDNA-SA-FasL over control groups including Exo-ssDNAbiotin at 72 h post cell infusion (FIGS. 14A-14B and FIGS. 15A-15B). The decrease in proliferating cell percentages resulted in significantly less absolute cell numbers of CD3+ and CD4+ T cells in the spleen and CD4+ T cells in lymph nodes (FIG. 2). Interestingly, CD4+ T cell proliferation was significantly inhibited in mice that received Exo-ssDNA-SA-FasL treatment but not in mice that received the same dose of soluble SA-FasL or native exosomes. These results showed that SA-FasL activity is significantly enhanced when immobilized on exosomes rather than used as freely soluble (“liquid-phase”) proteins. Collectively, the targeted delivery of SA-FasL via exosomes could substantially increase the therapeutic effect of SA-FasL protein while minimizing potential off-target effects caused by soluble injections of readily diffusible soluble SA-FasL.

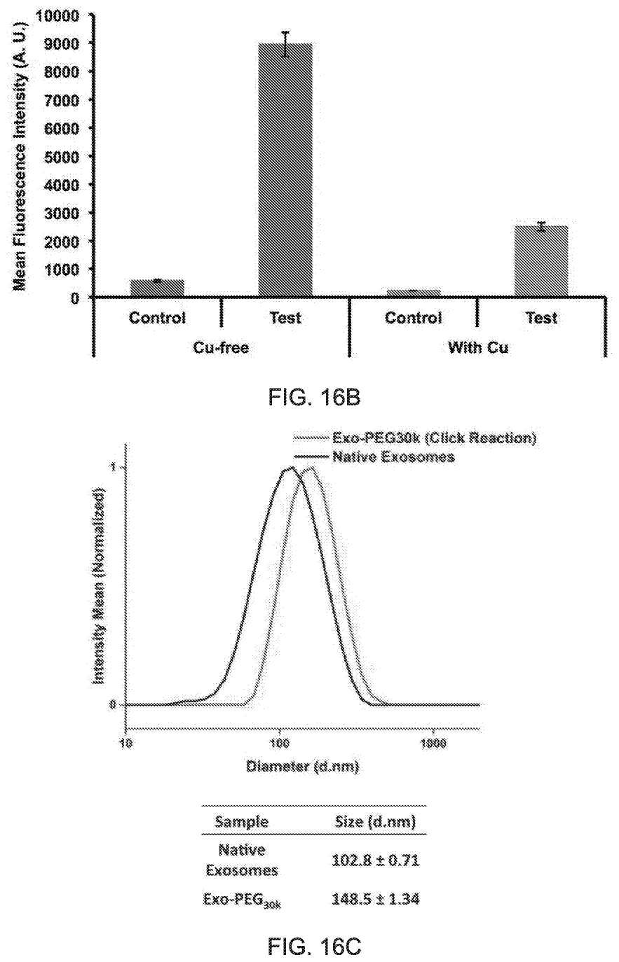

Click chemistry on exosomes: In order to expand on the versatility of this method for extracellular exosome functionalization, click chemistry was used to attach exemplary small dye molecules (Cy5 and SF488) and an exemplary polymer (PEG 30 k) to the exosome membrane, FIG. 16A. An Exo-dsDNA-N₃ was prepared by hybridizing a complementary DNA′-N₃ to the Exo-ssDNA. A Cu-free click linking chemistry was performed using dibenzo-bicyclo-octyne (DBCO) and a functionalized SF488 dye or PEG30 k polymer. The formation of the SF488-clicked exosomes were confirmed by flow cytometry analysis using 488 nm channel, while no significant signal was observed for control experiment, FIG. 16B. The PEG-clicked exosome polymer hybrid was analyzed by DLS, showing a significant increase in the size of the hybrid as compared to the native exosomes, FIG. 16C. Some non-specific attachment was observed in control experiments after incubation of native exosome and PEG-30 k-DBCO under exactly the same reaction conditions. Additionally, a Cu-click reaction was performed with Cy5-alkyne to test the robustness of this approach, FIG. 16A.

### Example 2: Engineering Exosome Polymer Hybrids (EPHs) Using Controlled Radical Polymerization

Polymers can provide a number of advantages associated with the increase in functional groups available for secondary interactions, derivatization, and changes in biochemical properties of exosomes. In the context of drug delivery, EPHs can be engineered for enhanced pharmacokinetics and bio-distribution profile compared to native exosomes. However, it is a critical requirement to maintain the surface profile of the functionalized exosome, since it is believed that the cellular uptake of exosomes occurs through cellular recognition of the surface molecules. Reversible deactivation radical polymerizations (RDRP) polymerization methods such as Atom Transfer Radical Polymerization (ATRP) and Reversible Addition-Fragmentation Chain-Transfer (RAFT) polymerization allows preparation of polymers with control over molecular weight and molecular weight distribution of the resulting polymer comprising radically (co)polymerizable monomers. Consequently, EPHs can be engineered with precise control over the length, composition, topology and functionality of the polymers that can be tethered to the membrane of exosomes. Indeed any polymer with a suitable terminal functionality can be incorporated into the w-functionalized oligonucleotide irrespective of its method of formation. RDRP procedures have been detailed in many papers and patents with one of the present inventors, K. Matyjaszewski, as primary author and are hereby incorporated by reference to provide details of the different procedures that can be employed to initiate and control the polymerization.

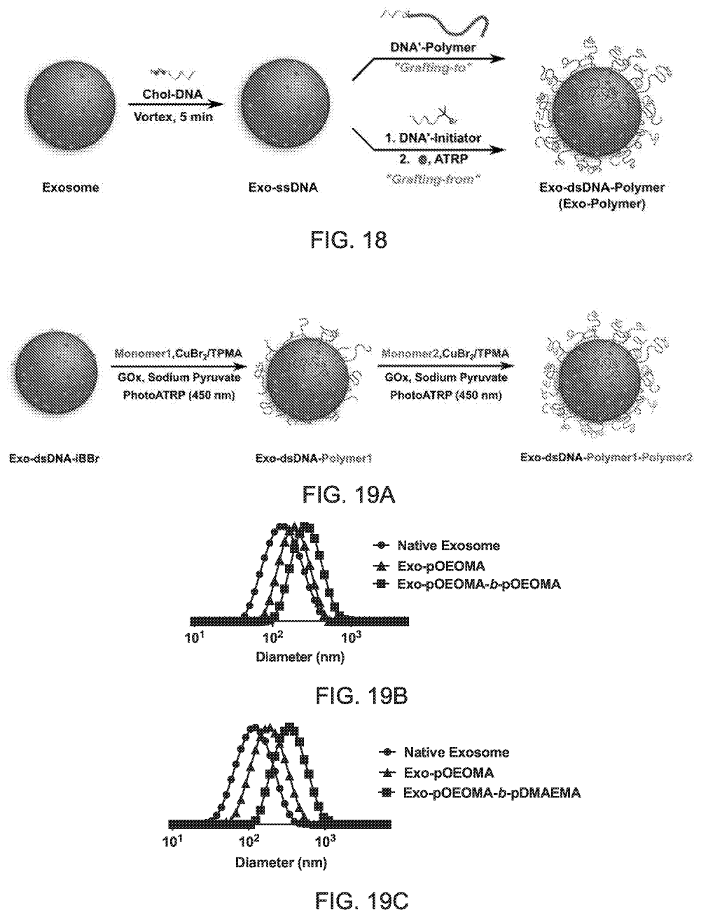

Here, the functionalization of exosome's surface with well-defined functional polymers using Atom Transfer Radical Polymerization (ATRP) is reported. Using a previously reported method for rapid and on-demand functionalization of exosomes by DNA tethers (Yerneni et al. “Rapid On-Demand Extracellular Vesicle Augmentation with Versatile Oligonucleotide Tethers”, ACS Nano 2019, 13(9):10555-10565), EPHs can be easily prepared by ‘grafting-to’ approach through hybridization of DNA block copolymers (FIG. 18). Alternatively, for ‘grafting-from’ approach, DNA ATRP macroinitiator can be tethered onto exosome surface allowing direct grafting of functional polymers from the exosome surface using biocompatible surface-initiated ATRP (FIG. 18). Our approach allows a precise control over the polymer loading on the exosome surface and we show that accessibility of surface proteins and membrane-tethered targeting agents-aptamer AS1411, can be easily modulated. The cellular uptake and bioactivity of engineered exosomes is preserved post-functionalization, while the stability of exosomes under different storage conditions as well as in the presence of proteolytic enzymes is significantly enhances. Our results show a significant enhancement in the blood circulation time of exosome polymer hybrids with preserved intrinsic tissue targeting properties.

### Materials and Methods

Cell culture: Mouse J774A.1 cells (ATTC® TIB-67™, Manassas) were grown and maintained in Roswell Park Memorial Institute medium (RPMI, Gibco, Gaithersburgh, Md.) supplemented with 10% heat-inactivated fetal bovine serum (FBS; Invitrogen, Carlsbad, Calif.) and 1% penicillin-streptomycin (Invitrogen, Carlsbad, Calif.). Mouse C2C12 cells (1% ATCC® CRL-1772™, Manassas, Va.) were grown in Dulbecco's Modified Eagle's Media (DMEM; Invitrogen, Carlsbad, Calif.) containing 10% FBS and 1% penicillin-streptomycin. Human umbilical vein endothelial cells (HUVECs; ATCC® CRL-1730™, Manassas, Va.) were grown and maintained in F-12K Medium supplemented with 10% FBS (Invitrogen, Carlsbad, Calif.), 0.1 mg/mL heparin (Millipore-Sigma, St. Louis, Mo.), 1% penicillin-streptomycin (Invitrogen, Carlsbad, Calif.) and endothelial cell growth supplement (BD Biosciences, Franklin lakes, NJ). RAW-Blue™ cells were grown and maintained in high-glucose DMEM supplemented with 10% HI-FBS, 1% PS and 100 μg/ml Normicin™ (Invivogen, San Diego, Calif.).

Exosome isolation and characterization: Exosomes were isolated and characterized as described in Example 1.

DNA Synthesis: DNA was synthesized as described in Example 1. The complementary 23-mer DNA macroinitiator (DNA′-iBBr) was synthesized by coupling isobromobutyrate initiator phosphoramidite on the 5′-end as previously reported (Averick et al. “Solid-Phase Incorporation of an ATRP Initiator for Polymer-DNA Biohybrids”, Angewandte Chemie International Edition, 2014, 53(10): 2739-2744). Additionally, Cyanine3-labelled DNA macroinitiator was synthesized using Cyanine3 (Cy3) CPG beads (Glen Research, Sterling, Va.). DNA′-AS1411 sequence was ordered from IDT (Integrated DNA Technologies, Inc., Iowa, USA) and used without any further purification.

Preparation of DNA Block Copolymer (DNABCp):

DNA′-pOEOMA: 50 μL of DNA′-iBBr (2 mM stock), 260 μL of OEOMA₅₀₀, 650 μL of the catalyst stock solution (12 mM CuBr₂, 72 mM Tris(2-pyridylmethyl)amine (TPMA)), 3.8 mL of ultrapure water and 250 μL of 1M NaCl were combined in a 20 ml glass vial. The reaction was degassed by passing a stream of nitrogen gas for 20 min. A 260 nm UV light source (5 mW/cm²) was used to start the polymerization by PhotoATRP. The reaction was carried out for 45 min. The reaction was analyzed by aqueous GPC and DNABCps were purified using ultra centrifugal 30 k MWCO filters before further usage. DNA′-pOEOMA strands of different polymer lengths (10 KDa, 20 KDa, 30 KDa) were synthesized by varying the reaction time. The resulting DNABCps were analyzed and purified before usage. Additionally, Cy3-modified DNA′-iBBr was used to prepare dye labeled DNABCPs for internalization studies.

DNA′-pCBMA: 25 μL of DNA′-iBBr (2 mM stock), 60 mg of CBMA (Carboxybetaine methacrylate), 266 μL of the catalyst stock solution (12 mM CuBr₂, 72 mM Tris(2-pyridylmethyl)amine (TPMA)), were mixed with 1.7 ml of 1×PBS buffer in a 5 ml glass vial. The reaction was degassed by passing a stream of nitrogen gas for 20 min. A 260 nm UV light source (5 mW/cm²) was used to start the polymerization by PhotoATRP. The reaction was carried out for 30 min, followed by analysis by aqueous GPC.

DNA′-pDMAEMA: 25 μL of DNA′-iBBr (2 mM stock), 45 μL of DMAEMA (Dimethylaminoethyl methacrylate), 266 μL of the catalyst stock solution (12 mM CuBr₂, 72 mM Tris(2-pyridylmethyl)amine (TPMA)), were mixed with 1.7 ml of 1×PBS buffer in a 5 ml glass vial. The reaction was degassed by passing a stream of nitrogen gas for 20 min. A 260 nm UV light source (5 mW/cm²) was used to start the polymerization by PhotoATRP. The reaction was carried out for 30 min, followed by analysis by aqueous GPC.

DNA′-pMSEA: 25 μL of DNA′-iBBr (2 mM stock), 40 mg of MSEA (2-(methylsulfinyl)ethyl acrylate)), 266 μL of the catalyst stock solution (12 mM CuBr₂, 72 mM Tris(2-pyridylmethyl)amine (TPMA)), were mixed with 1.7 ml of 1×PBS buffer in a 5 ml glass vial. The reaction was degassed by passing a stream of nitrogen gas for 20 min. A 260 nm UV light source (5 mW/cm²) was used to start the polymerization by PhotoATRP. The reaction was carried out for 45 min, followed by analysis by aqueous GPC.

Preparation of Exosome-Polymer Hybrids (EPHs) by “Grafting-To” Approach:

EPHs by annealing approach: 20 μg of isolated THP1 exosomes were gently vortexed (600 RPM, Scientific Industries Vortex-Genie 2 Vortex Mixer) with different concentrations of cholesterol-DNA (Chol-DNA; 0.1 μM to 20 μM) for 5 min at room temperature in 100 μL PBS buffer (final exosome concentration=0.2 μg/μL). The samples were then annealed with respective concentration of complementary polymer strand (DNA′-pOEOMA_{30K}) by sequentially incubation at 37° C., 0° C. and room temperature for 15 minutes, 10 minutes and 30 minutes respectively. Samples were then washed with Amicon Ultra Centrifugal Filters (100 k MWCO, Millipore Sigma, St. Louis, Mo.), followed by reverse spin to get Exo-dsDNA-pOEOMA (Exo-pOEOMA). Size and surface charge of the resulting species was measured using Zetasizer (Malvern Instruments Ltd, Malvern, UK).

EPHs by preannealing approach: Chol-DNA and complementary DNA′-pOEOMA_{30K} strand were annealed by sequential incubation at 37° C., 0° C. and RT for 15 min, 10 min and 30 min respectively. 20 μg of exosomes were then gently vortexed with different concentrations (from 0.1 μM to 20 μM) of preannealed duplex polymer strand (Chol-dsDNA-pOEOMA_{30K}). Samples were washed with 100 k MWCO filters to remove any excess polymer strand. Size and surface charge of the resulting species was measured using Zetasizer (Malvern Instruments Ltd, Malvern, UK).

Preparation of EPHs by “Grafting-From” Strategy:

Exosome Macroinitiator: Chol-dsDNA-iBBr was prepared by annealing Chol-DNA and DNA′-iBBr using procedure as described above. 60 μg exosomes were then gently vortexed with preannealed Chol-dsDNA-iBBr tether, followed by washes with Amicon Ultra Centrifugal Filters (100 k MWCO) to prepare Exosome macroinitiator (Exo-iBBr; 20 μM dsDNA tether concentration).

Atom Transfer Radical Polymerization: 150 μL of 60 μg Exo-iBBr, 7.5 μL of OEOMA₅₀₀, 20 μL the catalyst stock solution (12 mM CuBr₂, 72 mM Tris(2-pyridylmethyl)amine (TPMA)), 5 μL of Glucose Oxidase stock (15 mg/ml), 20 μL of Sodium Pyruvate stock (2 M), 15 μL of 10×PBS were mixed with 52.5 μL of H₂O. The reaction mixture was then transferred to a thin glass culture tube. 30 μL of glucose stock (1.5 M) was added and the vial was sealed for the deoxygenation (incubation for 5 min). The reaction vial was irradiated with blue light (4.5 mW/cm²) for 30 min. The reaction solution was washed with 100 k MWCO filters to get purified Exo-pOEOMA species, followed by analysis by dynamic light scattering.

Chain End Extension (Exosome Block Copolymer Hybrids): After the preparation of first block (Exo-pOEOMA) as described above, the reaction mixture was washed with 100 k MWCO filters and was reverse spun to a volume of 150 μL. For the chain extension, 150 μL Exo-pOEOMA, 7.5 μL of OEOMA₅₀₀ (or 10 μL DMAEMA), 20 μL the catalyst stock solution (12 mM CuBr₂, 72 mM Tris(2-pyridylmethyl)amine (TPMA)), 5 μL of Glucose Oxidase stock (15 mg/ml), 20 μL of Sodium Pyruvate stock (2 M), 15 μL of 10×PBS were mixed with 52.5 μL of H₂O. The reaction mixture was transferred to the glass vial and 30 μL of glucose stock (1.5 M) was added to start the deoxygenation. After 5 min, the reaction vial was irradiated with blue light for 30 min, followed by purification using 100 k MWCO filters. The purified Exo-pOEOMA-pOEOMA/pDMAEMA were analyzed using Zetasizer for size and surface charge.

Cytotoxicity studies: Cytotoxicity was assessed using direct CyQUANT® nucleic acid-sensitive fluorescence assay (Thermo Fisher Scientific, Waltham, Mass., USA) according to the manufacturer's instructions. Briefly, 25×10³ HEK293 cells/well were plated in 48-well microplate (Corning Inc., Corning, N.Y., USA) and allowed to adhere overnight. Treatments with varying concentrations of the purified Exo-pOEOMA species were added and co-incubated with cells for designated time-points. As controls, OEOMA₅₀₀ monomer and CuBr₂/TPMA catalyst were also assessed at concentrations used for the preparation of Exo-pOEOMA species. Next, cells were labeled with CyQUANT® Direct and fluorescence intensities were measured with TECAN spectrophotometer reader (TECAN, Männedorf, Switzerland). Cytotoxicity was assessed by normalizing fluorescence intensities to control group (no treatment) and plotted as percent viability.

Surface accessibility assessment of Exosome Polymer Hybrids by flow cytometry: DNA′-pOEOMA strands of different molecular weights (10 KDa, 20 KDa, 30 KDa) were synthesized and purified as described above. 20 μg Exo-dsDNA-Cy5-pOEOMA species were prepared by preannealing approach using varying ratios of Chol-dsDNA-Cy5 and Chol-dsDNA-Cy5-pOEOMA tethers. This in turn, kept the Cy5 concentration constant (10 μM) on all species, while pOEOMA loading was varied (0 μM, 0.1 μM, 0.5 μM, 1 μM, 5 μM) using complementary DNA′-pOEOMA of different molecular weights (10 KDa, 20 KDa, 30 KDa). The resulting EPHs were gently vortexed overnight at 4° C. with anti-CD63 conjugated magnetic streptavidin beads. Beads were washed with 1×PBS buffer three times (5 min each wash) to remove any unbound exosomes, followed by flow cytometry analysis on an Accuri C6 flow cytometer (BD Biosciences, San Jose, Calif.) connected to an Intellicyt HyperCyt autosampler (IntelliCyt Corp., Albuquerque, N. Mex.) using Cy5 channel (649 nm). Data were processed and interpreted using FlowJo® software (Flowjo LLC, Ashland, Oreg.).

Stability towards DNase-I: To assess the stability of DNA tethering on EPHs, Ant-CD63 beads-bound EPHs of varying polymer length and loading (as described above) were incubated with 2.5 units of DNase-I (New England Biolabs, Ipswich, Mass.) suspended in 1× DNase I Reaction Buffer (New England Biolabs, Ipswich, Mass.) for 15 min at 37° C. Post incubation, beads were magnetically separated and thoroughly rinsed in 1×PBS prior to flow cytometric analysis as described above.

Internalization studies: Native exosomes were labeled with PKH26 and PKH67 dyes (Sigma-Aldrich, St. Louis, Mo.) according to manufacturer's instruction. Briefly, exosomes were pellet by centrifugation (100,000×g for 3 hr) and resuspended in the diluent C. The respective dye was diluted (4:1000) in diluent C and added to the Exosomes followed by rigorous mixing for 90 seconds. The excess dye was quenched with 1% BSA in Diluent C for 5 min at RT and filtered using 300 K Da. M.W.C.O filter (Sartorius, Germany). Labeled exosomes were resuspended in 1×PBS and used for in vitro studies. Exosome samples for internalization samples were prepared with 1 μM DNA tether concentration by preannealing approach using PKH26/PKH67-labeled exosomes, Chol-DNA-Cy5 and Cy3-DNA′-pOEOMA (10 KDa, 20 KDa, 30 KDa) strands.

HEK293 cells were seeded at 2.5×10³ cells/cm² on collagen type-I coated coverslips (Electron Microscopy Services, Hatfield, Pa.) and allowed to adhere for 4 hr prior to the addition of labelled exosomes. To inhibit uptake, cells were pretreated with a combination of 10 μg/mL heparin (Sigma-Aldrich, St. Louis, Mo.) and 1 μM methyl-β-cyclodextrin (Sigma-Aldrich, St. Louis, Mo.) for 1 hr at 37° C. Exosomes were added to a final concentration of 20 μg/ml for designated time points. Post incubation, the plasma membrane bound Exosomes were washed-off using stripping buffer (500 μM NaCl and 0.5% acetic acid in DI water, pH: 3) for 1 min and cell were fixed in 3.33% paraformaldehyde (PFA; Electron Microscopy Services, Hatfield, Pa.) at room temperature (RT) for 15 min. Excess PFA was quenched with 1% bovine serum albumin (BSA; Sigma-Aldrich, St. Louis, Mo.) in PBS and the monolayer was rinsed thoroughly four times with 1×PBS. Cells were permeabilized with 0.1% Triton-X (Sigma-Aldrich, St. Louis, Mo.) for 1 min and stained for 10 min at RT with Alexafluor®647-phalloidin/Alexafluor®488-phalloidin (Invitrogen, Carlsbad, Calif.) diluted to 3:80 in 1×PBS. Nuclei was stained with 1:1000 (in 1×PBS) Hoechst 33342 (ThermoFisher Scientific, Waltham, Mass.) for 5 min, rinsed thoroughly and mounted using prolong gold (Invitrogen, Carlsbad, Calif.). Imaging was performed using Zeiss LSM 880 confocal microscope (Carl Zeiss Microscopy GmbH, Jena, Germany) under constant settings across all the different treatment groups and analyzed using Imaris microscopy analysis software (Bitplane AG, Zurich, Switzerland). The amount of exosome internalized was evaluated by comparing the relative fluorescence intensities measured in NIH ImageJ software post background subtraction. Five random pictures were captured per treatment group for the mean fluorescence measurement evaluations.

Cellular uptake of AS1411-functionalized Exosome Polymer Hybrids with different AS1411 loadings: EPHs with AS1411 and pOEOMA₃₀K were prepared by preannealing approach, using PKH26/PKH67-labeled exosomes, Chol-DNA, DNA′-pOEOMA and DNA′-AS1411 strands. Exo-pOEOMA-AS1411_{Low}: 20 μg of labeled exosomes were simultaneously vortexed with preannealed chol-dsDNA-pOEOMA_{30K} and chol-dsDNA-AS1411 at 1 μM concentration of both. The samples were next washed with 100K MWCO filters as described above. Exo-pOEOMA-AS1411_{High}: 20 μg of labeled exosomes were simultaneously vortexed with preannealed chol-dsDNA-pOEOMA_{30K} (1 μM) and chol-dsDNA-AS1411 (10 μM) concentration. The samples were next washed with 100K MWCO filters as described above. Internalization in of different species of Exo-pOEOMA-A51411 were performed with MiaPaCa2 and HEK293 in presence/absence of different inhibitors using the protocol described above.

Preparation of Exosome Polymer Hybrids by click chemistry: Chol-DNA and DNA′-N₃ were annealed using sequentially incubation at 37° C., 0° C. and room temperature for 15 minutes, 10 minutes and 30 minutes respectively. 100 μg of exosomes were gently vortexed with preannealed Chol-dsDNA-N₃ in to prepare Exo-dsDNA-N₃ (20 μM azide concentration). The sample was concentrated to 200 μM azide concentration using ultra centrifugal filters (MWCO=100 k) for click reaction. 25 μL of Exo-dsDNA-N₃ (200 μM azide concentration), 25 of PEG_{30K}-DBCO (1 mM stock) and 5 μL DMSO were mixed and incubated at 4° C. for 16 hours. The sample was washed using 100 k MWCO filters to remove unbound PEG_{30K}-DBCO, followed by analysis using dynamic light scattering. For the control experiment, 50 μg of native exosomes, were incubated with PEG_{30K}-DBCO under exact same conditions.

Exosomes with reversible polymer functionalization: Using photocleavable Chol-pc-DNA tether, Exo-pc-pOEOMA_{30K} were prepared by preannealing approach at a tether concentration of 1 μM. The resulting EPHs were analyzed by DLS for increase in the average diameter as compared to non-functionalized exosomes. Exo-pc-pOEOMA_{30K} were then irradiated with UV light for 2 min (50 mW/cm²) to cleave the polymer from the surface, followed by DLS analysis.

Stability Studies:

Under storage conditions: Exo-pOEOMA_{30K} (1 μM DNA tether loading) were prepared by preannealing approach as described above. Native exosomes and Exo-pOEOMA_{30K} (exosome concentration: 0.4 μg/μL) were incubated in 1×PBS buffer at different temperatures (4° C. and 37° C.) for a period of one month. The samples were analyzed by dynamic light scattering.

Against Trypsin: Exosomal surface proteins were radioactively labeled using Iodine¹²⁵.Radiolabeled native exosomes and EPHs with photocleavable tethers were treated with 0.25% trypsin (ThermoFisher Scientific, Waltham, Mass.) for 60 mins at 37° C. and were analyzed by size exclusion chromatography. Photocleavable EPHs were then irradiated with UV light for 2 min (50 mW/cm²) to cleave the polymer from the surface, followed by another incubation for 60 mins at 37° C. The samples were reanalyzed by size exclusion chromatography.

RAW-Blue assay: RAW-Blue™ cells (murine RAW 264.7 macrophage reporter cell line) were purchased from InvivoGen (San Diego, Calif.). This reporter cell line stably expresses a secreted embryonic alkaline phosphatase (SEAP) gene inducible by NF-kB activation that can be detected calorimetrically. The assay was performed according to manufacturer's instructions. Briefly, 20,000 RAW-blue cells and treatments consisting of 10 μg/ml Exo-pOEOMA_{30K} and/10 ng/ml LPS (positive control) were added to 96 well plates in triplicate and incubated for 24 h under culture conditions (37° C., 5% CO₂ and 95% relative humidity). Post incubation, 20 μI of conditioned media was collected and incubated with 200 μl QUANTI-Blue™ reagent (Invivogen, San Diego, Calif.) and optical density at 655 nm was measured using TECAN spectrophotometer (TECAN, Männedorf, Switzerland).

Angiogenesis studies: Tube formation assay was done as previously described (Ludwig et al. “HNSCC-derived exosomes promote angiogenesis through reprogramming of 1 endothelial cells in vitro and in vivo”, 2018, Mol Cancer Res, 0358). HUVECs and rat lymph endothelial cells (2×10⁴) were re-suspended in serum-free media and placed on top of 70 μL growth factor-reduced Matrigel (Corning Inc., Corning, N.Y.) in wells of 48-well plates. Cells were treated with 10, 20 or 50 μg of TEX per well. Following incubation at 37° C. for 6 h, tubules were imaged in 5 random regions of interest, using phase contrast microscopy at 10× magnification (Axiovert 25 CFL, Carl Zeiss Microscopy). Tubule length and numbers of branch points were analyzed with the Angiogenesis Analyzer developed for the ImageJ software.

Alkaline phosphatase (ALP) assay: C2C12 cells were incubated with indicated treatments, washed with PBS to remove culture medium, and fixed for 20 min with 10% neutral buffered formalin (Millipore-Sigma, St. Louis, Mo.). Alkaline phosphatase activity was detected using a leukocyte alkaline phosphatase assay kit according to the manufacturer's instructions (Millipore-Sigma, St. Louis, Mo.). Where required, ALP-stained images were converted to CMYK format since this color format is representative of reflected light colors as opposed to emitted light colors (RGB). Since the combination of cyan and magenta form the color blue, these channels were added together and inverted. The average pixel intensity was determined using the image histogram tool in Adobe® Photoshop 7.0 (Adobe® Systems, San Jose, Calif.).

In vivo blood circulation studies: C57BL/6 male mice (n=8; 22-26 grams) were utilized for blood circulation studies. Animal care and experimental procedures were carried out at Carnegie Mellon University (Pittsburgh, Pa.) in accordance with the NIH Guide for the Care and Use of Laboratory Animals under an approved Institutional Animal Care and Use Committee (IACUC) protocol. Freshly purified 40 ug of near-IR ExoGlow-labeled EVs (ExoGlow™, System Biosciences, Palo Alto, Calif.) were injected through the tail vein for intravenous (i.v.) injections. At indicated time-points, ˜10-40 μI of blood was drawn from the mice using submandibular bleeding technique. 10 ul blood was heparinized and the amount of fluorescence from near-IR ExoGlow-labeled EVs was quantified using TECAN (TECAN plate reader, Männedorf, Switzerland) using excitation of 784 nm and emission of 806 nm.

In vivo biodistribution studies: C57BL/6 male mice (n=6; 22-26 grams) were utilized for tissue distribution studies based on a previously published protocol (Wiklander et al. “Extracellular vesicle in vivo biodistribution is determined by cell source, route of administration and targeting”, Journal of extracellular vesicles, 2015, 4: 26316-26316). Animal care and experimental procedures were carried out at Carnegie Mellon University (Pittsburgh, Pa.) in accordance with the NIH Guide for the Care and Use of Laboratory Animals under an approved Institutional Animal Care and Use Committee (IACUC) protocol. Freshly purified 40 ug of ExoGlow-labeled EVs (ExoGlow™, System Biosciences, Palo Alto, Calif.) were injected through the tail vein for intravenous (i.v.) injections. 24 hours after injection mice were sedated and the vascular system was flushed by transcardial perfusion for 5 minutes following which the animals were euthanized. Organs were harvested and imaged using IVIS Spectrum (PerkinElmer, Waltham Mass.) using excitation of 710 nm and emission of 760 keeping all the other settings constant. The data were analyzed with the IVIS imaging system software.

### Results and Discussion

Preparation of Exosome Polymer Hybrids by Grafting-to Strategy:

Preparation of DNA Block Copolymer: A 23-mer complementary DNA stand was developed as ATRP macroinitiator (DNA-iBBr) to graft-polymer using PhotoATRP. Different DNABCp were synthesized with OEOMA₅₀₀ as monomer, with varying degrees of polymerization, followed by purification using 30 k MWCO filter. The polymerization conditions and results are summarized in Table 2 below.

TABLE 2
DNA Block Copolymers synthesis using Photo-ATRP

| | [OEOMA₅₀₀]/ | | | |
|---|---|---|---|---|
| | [I]/[CuBr₂]/[TPMA] | Time | Mₙ^{a} | M_{w}/Mₙ^{a} |
| DNA-iBBr | — | — | 7,000 | 1.01 |
| DNABCp-30k | 5500/1/80/480 | 40 min | 40,000 | 1.14 |
| DNABCp -20k | 5500/1/80/480 | 25 min | 32,000 | 1.14 |
| DNABCp -10k | 5500/1/80/480 | 15 min | 19,000 | 1.06 |

Polymerization conditions are DNA-iBBr as an initiator, [I] = 20 μM, 50 mM NaCl, total volume: 5 ml PhotoATRP (UV lamp),
^{a}Using PEO standards.

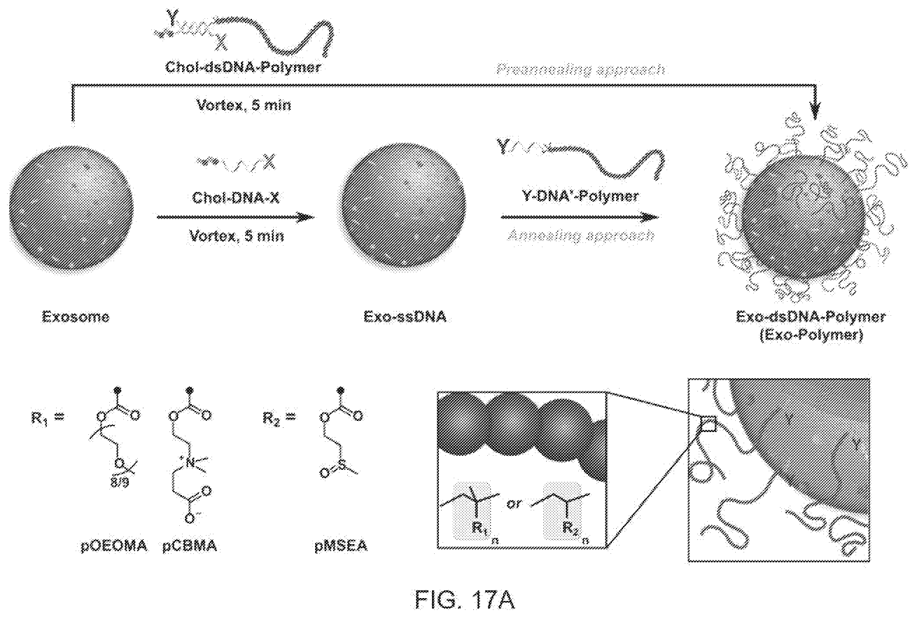

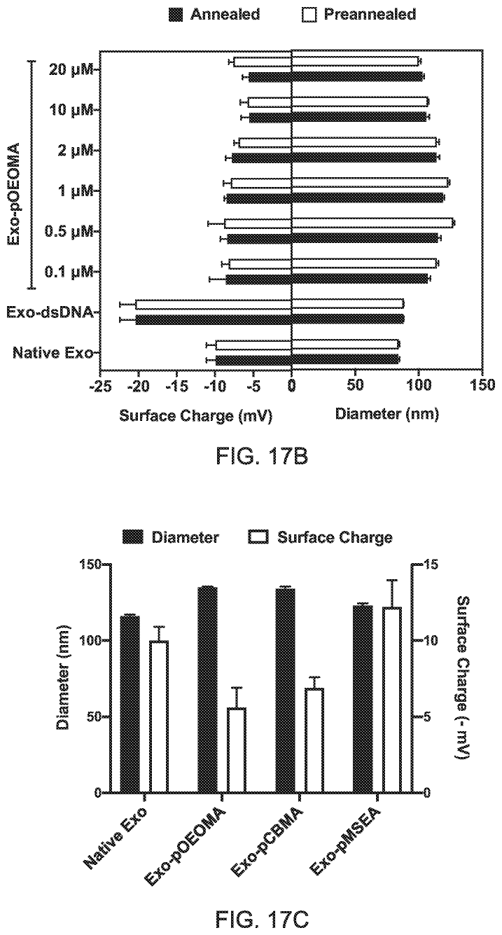

In order to attach polymers on the exosome surface, we used our previously reported method for exosome membrane functionalization through DNA tethers (Yerneni et al. “Rapid On-Demand Extracellular Vesicle Augmentation with Versatile Oligonucleotide Tethers”, ACS Nano 2019, 13(9):10555-10565). A 18-mer DNA tether, functionalized with cholesterol and triethyleneglycol units as spacers on the 5′-end (Chol-DNA) is gently vortexed with exosomes to prepare DNA tethered exosomes (Exo-ssDNA). This DNA tether on the exosome surface serves as a handle to anneal complementary DNA block copolymers (DNABCPs; DNA′-Polymer), generating exosome polymer hybrids (EPHs) by annealing approach (FIG. 17A). Alternatively, Chol-DNA can be annealed with the complementary DNA′-Polymer before tethering to the exosome surface, generating EPHs by preannealing approach (FIG. 17B). Both these grafting-to strategies for EPHs allow the preparation of well-defined DNABCPs with known compositions. Complementary DNABCPs were prepared using a 23-mer DNA macroinitiator (DNA′-iBBr), with 5′-α-bromoisobutyrate group using previously reported method (Averick et al.). Using oligo(ethylene oxide) methacrylate (OEOMA, Mₙ=500) as monomer and DNA-iBBr as initiator, DNA′-pOEOMA is synthesized by photo-induced ATRP (PhotoATRP). Varying the concentration of Chol-DNA and DNA′-pOEOMA strands (from 0.1 μM to 20 μM) with same number of exosomes, allows the preparation of Exo-pOEOMA species with varying polymer loading. Both annealing and preannealing approach showed an increase in the average diameter of the vesicles after polymer functionalization (FIG. 17B). Surface charge of the resulting EPHs showed comparable surface charge as native exosomes (FIG. 17B).

Further, to increase the monomer scope, we prepared EPHs with different biocompatible polymers. EPHs with zwitterionic polymers were prepared using carboxybetaine methacrylate (CBMA) as the monomer. Additionally, we explored dimethyl sulfoxide-derived biocompatible polymer—poly(2-(methylsulfinyl)ethyl methacrylate (pMSEA), to prepare the EPHs (FIG. 17C). However, EPHs with cationic polymers using grafting-to approach is challenging due to electrostatic interactions of polymer with the negatively charged membrane, which can interfere with the tethering efficiency of Chol-DNA. DLS data showed multimodal distribution for the EPHs prepared using cationic DMAEMA (2-(Dimethylamino) ethyl methacrylate) monomer. These results motivated us to explore alternative grafting-from strategy and prepare EPHs with increased monomer scope.

Preparation of Exosome Polymer Hybrids by Grafting-from Strategy:

In order to graft the polymer from exosome macroinitiator, PhotoATRP is not the appropriate technique therefore initially AGET ATRP (Activators are Generated by Electron Transfer) conditions were examined with low initiator concentrations due to limited amounts of available exosome and DNA.

Functionalization of exosomes with ATRP initiator facilitates the preparation of EPHs by grafting well-controlled polymers directly from the exosome surface. This strategy mandates biocompatible polymerization conditions to preserve the integrity of exosomes. All CRP methods are inhibited by the presence of oxygen and requires rigorous degassing procedures such as ‘free-pump-thaw’ cycles. Hence, glucose oxidase (GOx)-mediated oxygen tolerant ATRP was used, which in the presence of glucose and sodium pyruvate, converts oxygen to carbon dioxide (Enciso et al. “A Breathing Atom-Transfer Radical Polymerization: Fully Oxygen-Tolerant Polymerization Inspired by Aerobic Respiration of Cells”, Angewandte Chemie International Edition, 2018, 57(4):933-936. Further, blue light-mediated PhotoATRP was used to avoid exposure of exosomes to ultra-violet (UV) irradiation (Fu et al. “Synthesis of Polymer Bioconjugates via Photoinduced Atom Transfer Radical Polymerization under Blue Light Irradiation”, ACS Macro Letters, 2018, 7(10):1248-1253). Exosome macroinitiator species, prepared Chol-DNA and complementary DNA′-iBBr using preannealing approach, were used to graft well-controlled polymers from the exosome surface. OEOMA₅₀₀ was chosen as the monomer and CuBr₂/TPMA (TPMA=Tris(2-pyridylmethyl)amine) as the catalyst in PBS buffer (pH 7.4). The polymerization was performed in the presence of glucose, GOx and sodium pyruvate by irradiating blue light (4.5 mW/cm²) for 30 minutes (FIG. 19A). DLS measurements showed a clear shift in the size of the particles. In order to further confirm the living nature of the polymerization process, the chain extension experiment was performed to graft second block of pOEOMA from the purified Exo-pOEOMA species (FIG. 19B). More significantly, EPHs with cationic polymers were also prepared by grafting a second block of pDMAEMA from Exo-pOEOMA species (FIG. 19C).

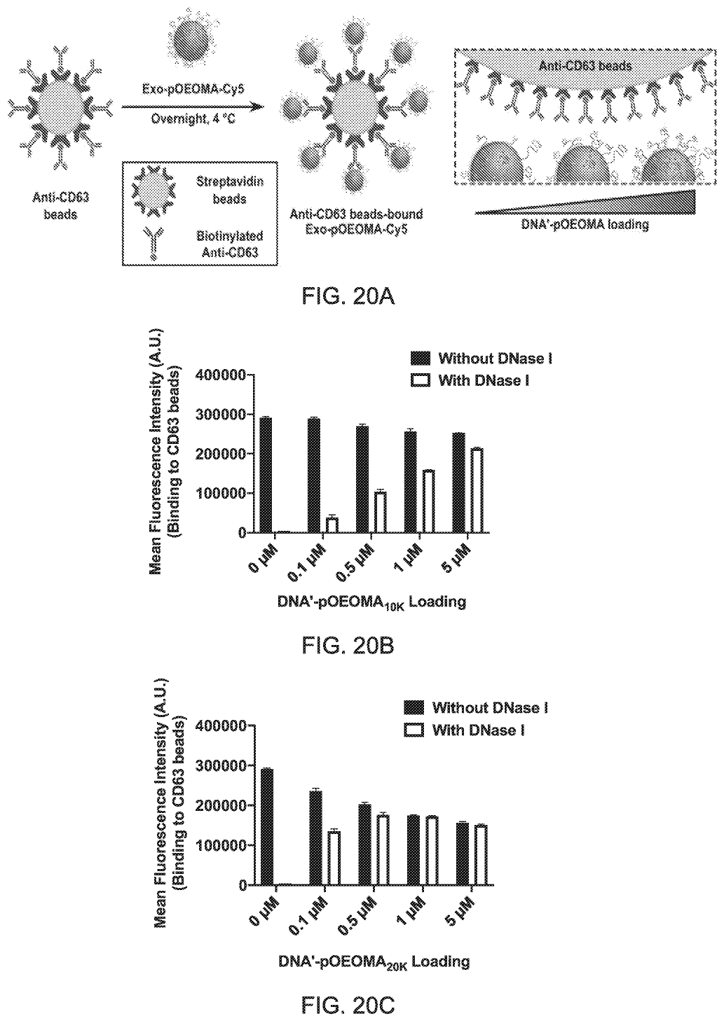

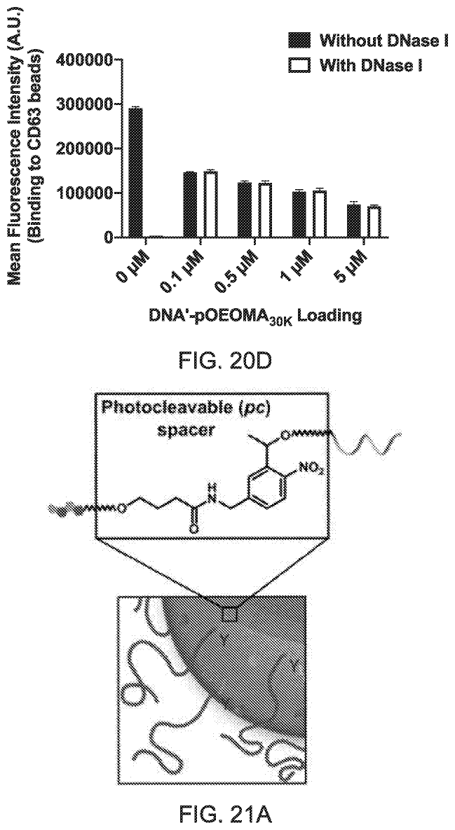

Analysis of accessibility of exosomal surface proteins and DNA tethers: Attachment of polymers on the exosome surface can affect the accessibility and hence the functions of surface proteins, rendering them biologically less affective. To investigate the effect of polymers, the accessibility of an exosomal surface marker protein CD63 was assessed through flow cytometry (FIG. 20A). Binding of cyanine5-labeled EPHs to the anti-CD63 magnetic beads acted as the parameter for accessibility analysis. Exo-pOEOMA species were prepared with varying surface loading of different MWs of polymer using DNA′-pOEOMA (10K, 20K, 30K). Using preannealing approach, EPHs samples were prepared with constant loading of Chol-DNA-Cy5 (10 μM) and different loading of DNA′-pOEOMA strand (0-5 μM). A decrease in the accessibility of CD63 protein was observed with increasing polymer MWs and surface loading (FIGS. 20B-20D). EPHs with pOEOMA_{10K} showed similar binding efficiency to the beads as exosomes without polymer strand and no significant effects of polymer loading was observed. However, Exo-pOEOMA_{20K} and Exo-pOEOMA_{30K} showed around 40% and 65% decrease in the binding efficiency respectively. A clear trend in the decrease of Exo-pOEOMA binding to the beads was observed with increase in the surface polymer loading for 10K and 20K polymers.

To further investigate the nuclease stability of DNA tethers, anti-CD63 beads-bound Exo-pOEOMA were treated with DNase-I enzyme. Exo-pOEOMA_{30K} showed complete protection against DNase-I even at the minimum polymer loading of 0.1 μM (1% with respect to Chol-DNA-Cy5 strand) (FIG. 20D). Exo-pOEOMA_{20K} and Exo-pOEOMA_{10K} show increase in nuclease stability with increasing polymer loading (FIGS. 20B and 20C). For the control exosome samples with no polymer, a complete cleavage of Chol-DNA-Cy5 was observed, highlighting the effect of the polymers towards nuclease stability of DNA tethers. Taken together, our results revealed that for polymers of 20K and higher, surface loading at the concentration of 1 μM is optimum for surface accessibility and nuclease stability of DNA tethers. These results are crucial to understand and modulate the effect of polymer on the surface functionality of exosomes.

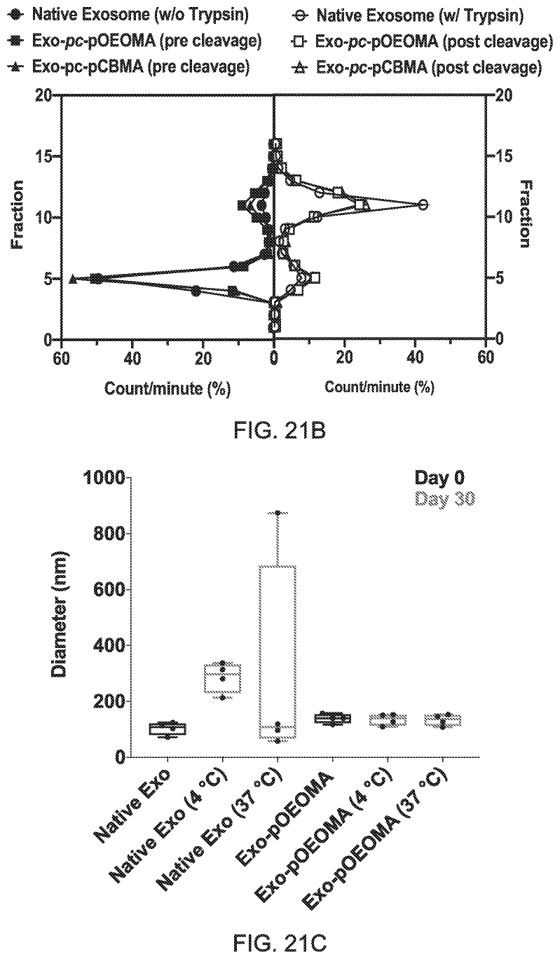

Exosomes with reversible polymer functionalization: In order to achieve a temporal control on the polymer functionalization, a photocleavable DNA tether (Chol-pc-DNA) with p-nitrophenol group was synthesized between the DNA and the 5′-cholesterol moiety (FIG. 21A). EPHs with photocleavable tether (Exo-pc-pOEOMA) allowed reversible functionalization of exosomes with polymers, showing complete removal in 2 minutes of UV light irradiation.

DNA Tether Stability Studies:

Stability of EPHs towards DNAse In order to study the accessibility of DNAse or other proteins towards exosome surface as well as to study the stability of DNA tethering, bead-bound EPHs were treated with DNAse. The 30 k polymer provided complete protection against DNase with the bead bound EPHs retaining 100% fluorescence, even at the minimum polymer loading (1% with respect to anchor strand). EPHs with 20 k and 10 k polymers show increase in DNase stability with increasing polymer loading (1%→50%). For the control experiment, i.e., exosomes with no polymer strand, a complete cleavage of DNA was observed, highlighting the stabilization effect of the polymers.

Internalization studies of EPHs In order to assess the effect of polymer towards internalization inside the cells, EPHs with different polymer length were tested for internalization in HEK 293 cells. EPHs with all polymer lengths were internalized into the HEK 293 cells, suggesting no significant effect of polymers on the ability for internalization. However, a relative drop was observed in internalization efficiency with increasing lengths of the polymer.

Examination of effect of polymer on the storage stability of EPHs A month long study of the effect of polymers on exosome storage stability was conducted at three different storage temperatures: 4° C., 37° C. and −20° C. The native exosome started aggregating while no aggregation was observed for the EPHs.

Stability studies with Trypsin Stability of Exo-POEOMA (1 uM loading) was explored against trypsin. EPHs are stable in the presence of trypsin, while native exosomes tend to clump and shed surface proteins after 48 hours. These studies were performed at two different temperatures of 4° C. and 37° C.

Exosomes with enhanced stability: Using the photocleavable tethers, we looked into the effect of polymer functionalization on the stability of exosomal surface proteins against proteases. DLS measurements of Exo-pc-pOEOMA with trypsin showed no change in the size profile of the hybrids after 24 hours; on the contrary, native exosomes showed aggregation as well as protein population around 10 nm region. To probe further and for better sensitivity, exosomal surface proteins were radiolabeled the using I¹²⁵. Radiolabeled native exosomes and EPHs with photocleavable tethers were treated with Trypsin for 60 mins at 37° C. and were analyzed by size exclusion chromatography. Native exosomes with trypsin showed a clear shift in the radioactivity from exosomes (fraction 10-12) to protein population (fraction 4-6), highlighting protein degradation. On the contrary, Exo-pc-pOEOMA and Exo-pc-pCBMA showed major radioactivity in the exosome fractions, suggesting enhanced stability of surface proteins towards trypsin. Extended incubation of EPHs samples after irradiating them UV light for 2 min, showed protein degradation after 60 min (FIG. 21B). These results highlight the reversible polymer protection of surface protein against trypsin.

Limited stability of exosomes under storage conditions motivated us to examine the effect of polymers on long-term storage (Lee et al.). Native exosomes and Exo-pOEOMA with 1 μM loading were incubated for one month at different temperatures. DLS measurements showed aggregation of native exosomes at 4° C., while shedding of exosomal surface proteins was observed at 37° C. (FIG. 21C). These observations are in agreement with previously reported studies (Armstrong et al. “Re-Engineering Extracellular Vesicles as Smart Nanoscale Therapeutics” ACS Nano, 2017, 11(1):69-83). Interestingly, EPHs showed a monomodal distribution even after one-month incubation with no aggregation or protein shedding at 4° C. or 37° C. (FIG. 21C).

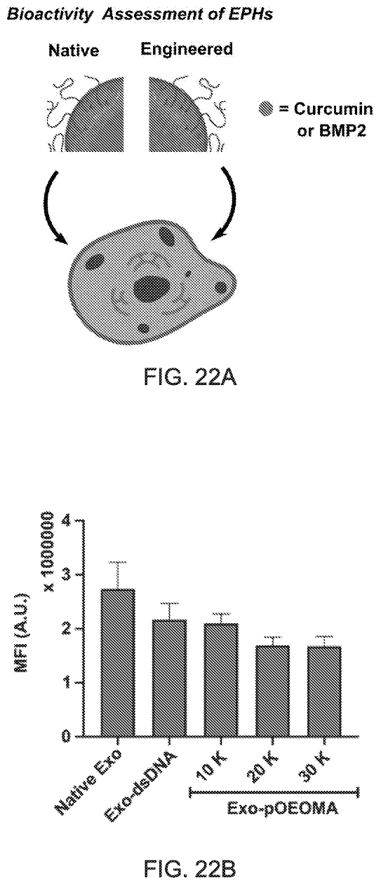

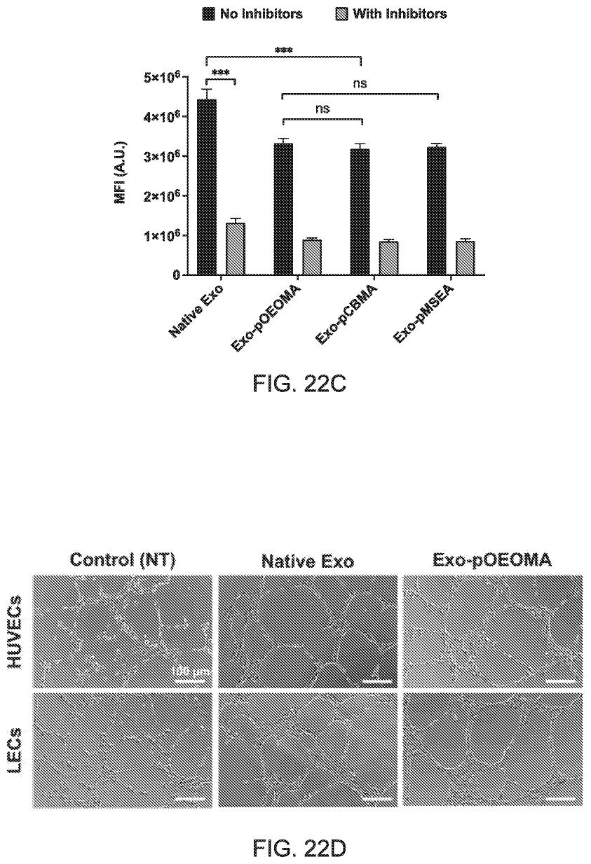

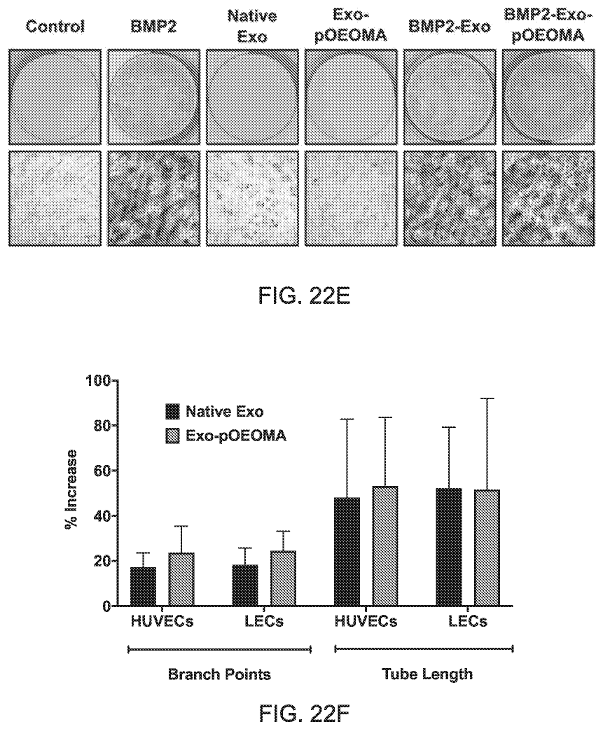

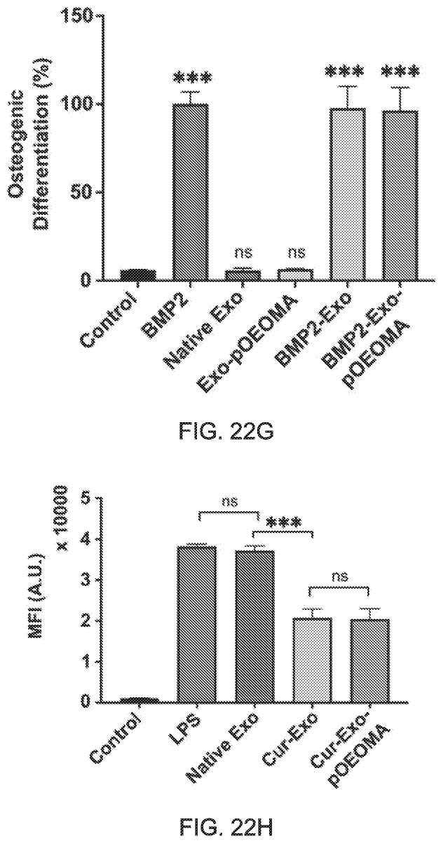

Effects of polymer functionalization on the cellular uptake of exosomes: Excessive surface functionalization of exosomes can easily render them biologically useless or less efficient. Based on our surface accessibility results, we probed into intrinsic biological properties of native and exosome polymer hybrids with 1 μM polymer loading. Firstly, we performed the cellular uptake studies of dye-labeled Exo-pOEOMA in human embryonic kidney (HEK293) cells. Cells were incubated with native exosomes and Exo-pOEOMA for three different polymer lengths (10K, 20K, 30K). We observed that all Exo-pOEOMA samples internalize in 6 hours, however approximately 20% drop in the internalization efficiency was observed for Exo-pOEOMA_{30K} (FIG. 22B). Further, we compared the effect of different polymers of similar MWs on the cellular uptake of exosomes. EPHs with zwitterionic pCBMA (Exo-pCBMA_{30K}), pOEOMA (Exo-pOEOMA_{30K}) and DMSO-based pMSEA polymer (Exo-pMSEA) were incubated with HEK293 cells for 6 hours. These cell internalization trials were also performed in the presence of two inhibitors heparin and methyl-β-cyclodextrin, which partially inhibit exosome internalization by blocking heparin sulfate proteoglycans and lipid-raft-mediated processes, respectively (Ludwig et al. “HNSCC-derived exosomes promote angiogenesis through reprogramming of 1 endothelial cells in vitro and in vivo”, 2018, Mol Cancer Res, 0358). We observed that all EPHs species internalized with similar efficiency. In the presence of inhibitors, we observed a significant drop in internalized fluorescence of both native exosomes and EPHs, highlighting similar internalization mechanisms (FIG. 22C). Due to other potential pathways for internalization of exosomes, complete inhibition was not observed in the presence of inhibitors.

Effects of Polymer Functionalization on the Bioactivity of Exosomes:

Angiogenesis. Mesenchymal stem cell exosomes are known to have angiogenic properties (Liang et al. “Exosomes secreted by mesenchymal stem cells promote endothelial cell angiogenesis by transferring miR-125a”, Journal of Cell Science, 2016, 129(11):2182) and therefore we decided to evaluate the effects of polymer conjugation on their angiogenic capacity. HUVECs and LECs were treated with MSC-derived Exo-pOEOMA and native MSC-derived exosomes and analyzed for tube length and number of branch points. Both, native exosomes and Exo-POEOMA increased the tube length in HUVECs by 34% and 40% as compared to control (no treatment). In LECs, tube length increased by 40% and 34% by native exosomes and Exo-POEOMA. Similarly both treatments also increased the branch points by 15% and 20% in HUVECs and 16% and 22% in LECs as compared to control (no treatment) as shown in FIG. 22F. In summary, the results indicate that OEOMA grafting did not affect the angiogenic potential of MSC exosomes.

Osteogenic differentiation: The osteogenic properties of bone morphogenetic protein 2 (BMP2)-exosomes have previously been studied and therefore it was decided to evaluate the effect of polymer conjugation on osteogenic capacity of BMP2-exosomes. The bioactivity of BMP2-exosomes was evaluated by assessing the induction of alkaline phosphatase (ALP) in C2C12 cells after treatment with exosomes. ALP is one of the early osteogenic differentiation marker. The results show that the ALP upregulation in C2C12s treated with BMP2-Exo and BMP2-Exo-POEOMA were not significantly different from each other (FIG. 22G), indicating polymer grafting does not affect the biological activity of BMP2-exosomes.

Anti-inflammatory properties. Exosomes isolated from J774A.1 cells were loaded with bovine serum albumin followed by curcumin and their potential to downregulate NFkB expression in RAW-Blue™ macrophage cell line was evaluated with or without polymer grafting to compare their biological activities. This reporter cell line stably expresses a secreted embryonic alkaline phosphatase (SEAP) gene inducible by NF-kB activation that can be detected calorimetrically. The results indicate that both native J774A.1-derived curcumin-exosomes and J774A.1-derived curcumin-Exo-POEOMA counteracted\NF-kB activation by bacterial lipopolysaccharide (LPS) as shown in FIG. 22H, which was significantly lower compared to control (no treatment). Moreover, there was no significant difference in NF-kB activation between native J774A.1-derived curcumin-exosomes and J774A.1-derived curcumin-Exo-POEOMA treatments, suggesting that the biological activity of curcumin-loaded exosomes can be preserved after polymer grafting.

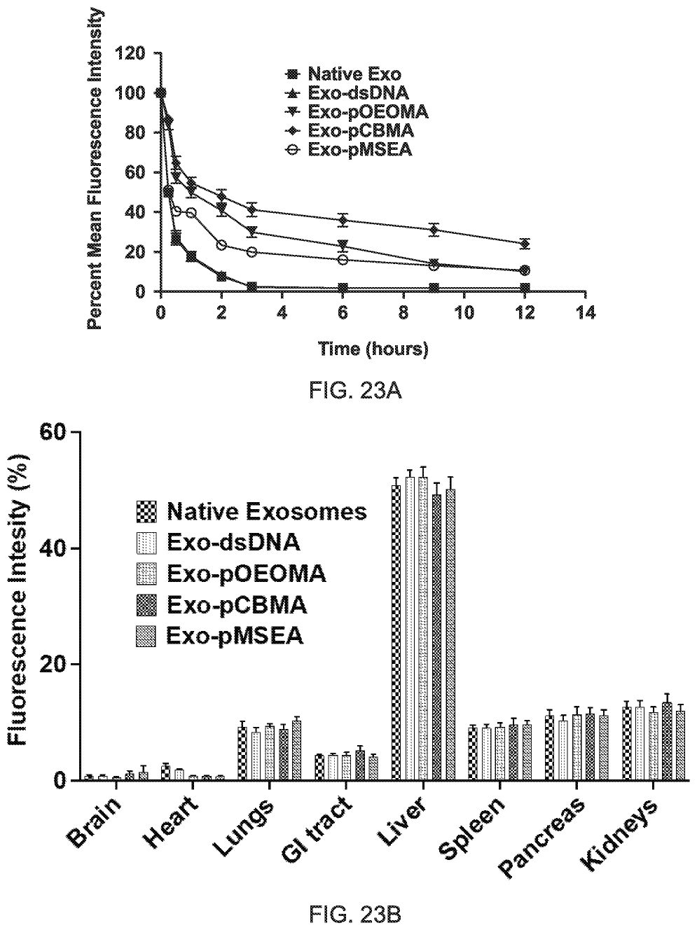

Pharmacokinetics, blood circulation and biodistribution: Exosome serum clearance kinetics was assessed by quantifying the ExoGlow signal in the mouse bloodstream as a function of time (FIG. 23A). Exosomes were labeled with ExoGlow according to manufacturer's instruction prior to polymer grafting. Three different types of polymers were evaluated: pOEOMA, pCBMA, and pDMSO at loading of 1 μM. Time zero draw was done around 15-30 sec (as quick as possible) post injection though it should be noted that maximum signal could have been there before that. Almost half of the fluorescence signal from native exosomes detected at time zero (15-30 sec) was distributed into tissues around 30 minutes. Half of the native exosomes initially detected at time zero had distributed to tissues by 15 min. At 120 min post injection, 7.5% of native exosome signal remained whereas at 180 min, only 2.4% signal remained. By contrast all the three polymer conjugated exosomes showed higher blood circulation time. Approximately 50 of the initially injected exosomes signal was detected at approximately 1 hour for Exo-POEOMA, at 2 hour for Exo-pCBMA and at 15 min for Exo-pDMSO. At 3 hours, almost 29.9% of Exo-POEOMA, 41% of Exo-pCBMA and 19.9% of pDMSO remained in blood circulation. Even after 12 hours, 10.2% of Exo-POEOMA, 24.1% of Exo-pCBMA and 10.7% of pDMSO remained in blood circulation.

For biodistribution experiments, exosomes were labeled with ExoGlow. Whole-organ IVIS images showed that native exosomes accumulated in lungs (9.28%), liver (50.9%), pancreas (11.2%) and kidney (12.8%), while that in brain (0.9%) and heart (2.5%) were low as shown in FIG. 23B. The tissue distribution profile of exosomes conjugated with different polymers was similar to that of native exosomes suggesting that although polymer-conjugation improves exosome blood circulation time, they do not change tissue distribution profile of exosomes.

Analysis of Surface Accessibility of EPHs by Flow Cytometry:

Surface accessibility of EPHs was indirectly analyzed in terms of the accessibility of a surface protein (CD63) by flow cytometry experiment. The experiment was designed to study the binding of exosomes on the anti-CD63-bound magnetic beads, which only binds when the surface protein, CD63, is accessible to the anti-CD63 present on the beads. The presence of POEOMA on the exosome membrane will decrease the binding to the CD63-modified magnetic beads suggesting lower surface accessibility of EPHs.

Different EPHs samples were prepared using the pre-annealing approach with constant concentration of Cy5 dye-labeled anchor strand, but with varying concentration of polymer strand (DNABCp), hence varying the overall polymer loading on EPHs. DNABCps of three different polymer lengths were used for the samples to determine the effect of the polymer length on the properties of the polymer hybrid exosome.

Flow cytometry studies were performed using Cy5 channel (649 nm) and results were compared to exosomes with no polymer strand, hence with complete surface accessibility. A linear decrease in the surface accessibility of EPHs was observed with increasing polymer loading. Additionally, the presence of a higher molecular weight polymer, 30 k, provided more surface coverage as compared to lower polymer length; as expected.

### Example 3: Exosome Gels by Atom Transfer Radical Polymerization

Previous examples have shown the preparation of exosome polymer hybrids by grafting polymers directly from the surface of exosomes using DNA tethers. Here, the preparation of exosome tethered gels using atom transfer radical polymerization (ATRP) is provided. It is demonstrated that exosome tethering in the gel network by noncovalent interactions provides a slower release profile as compared to directly trapped exosomes. Furthermore, osteogenic differentiation is demonstrated using this approach by highly controlled delivery of BMP2-EVs in vitro. BMP2 was selected as a paradigm growth factor to investigate because of its biological and clinical relevance.

### Materials and Methods

Cell Culture: Mouse J774A.1 cells (ATTC® TIB-67™, Manassas) were grown and maintained in Roswell Park Memorial Institute medium (RPMI, Gibco, Gaithersburgh, Md.) supplemented with 10% heat-inactivated fetal bovine serum (FBS; Invitrogen, Carlsbad, Calif.) and 1% penicillin-streptomycin (Invitrogen, Carlsbad, Calif.). Mouse C2C12 cells (ATCC® CRL-1772™, Manassas, Va.) were grown in Dulbecco's Modified Eagle's Media (DMEM; Invitrogen, Carlsbad, Calif.) containing 10% FBS and 1% penicillin-streptomycin. MC3T3-E1 subclone 4 cells (ATCC® CRL-2593™, Manassas, Va.) were grown in ascorbic acid-free α-minimum essential media (αMEM, Gibco, Gaithersburgh, Md.) media supplemented with 10% FBS and 1% PS. In all cell culture experiments, exosome-depleted FBS obtained by centrifugation at 100,000×g for 2 hr was utilized. Conditioned media was collected every 72 hr and stored at −80° C. if not used immediately for exosome isolation.

Exosome Isolation: Exosomes were isolated as described in Example 1.

Exosome Characterization: Exosomes were characterized using the methods described in Example 1, including DLS, TRPS, TEM, and Western Blotting.

DNA synthesis: DNA was synthesis as described in Example 1. The complementary 23-mer DNA macroinitiator (DNA′-iBBr) was synthesized by coupling isobromobutyrate initiator phosphoramidite on the 5′-end as previously reported (Averick et al.).

BMP2-Exo preparation and characterization: 10 μg of exosomes and 1 μg BMP2 mixture was sonicated (Tekmar sonic disruptor) on ice using a 0.25 inch tip at 20% amplitude, 6 cycles of 30 s on/off for three minutes with a 2 min cooling period between each cycle. The unloaded BMP2 was removed using a 100,000 kDa MWCO membrane filter (Vivaspin® columns, Sartorius AG, Göttingen, Germany). Exosome surface-bound BMP2 was removed by pH 3.0 acid-incubation followed by separation of exosomes from BMP2 using mini-SEC. To confirm the loading of BMP2 in exosomes, western blotting analysis was performed.

### Exosome Macroinitiator Preparation (Non-Cleavable and Photocleavable):

Exosome Macroinitiator: Chol-dsDNA-iBBr was prepared by annealing Chol-DNA and DNA′-iBBr using sequential incubation at 37° C., 0° C. and RT for 15 min, 10 min and 30 min respectively. 1004 of Exosomes or BMP2-Exosomes (0.4 μg/μL exosome concentration) were then gently vortexed with 100 μL of preannealed Chol-dsDNA-iBBr tether (2 μM dsDNA tether concentration), followed by three washes with Amicon Ultra Centrifugal Filters (100 k MWCO). The filters were reverse spun to prepare 100 μL of Exo-dsDNA-iBBr (0.4 μg/μL exosome stock concentration, 1 μM initiator concentration).

Photocleavable (pc) Exosome Macroinitiator: Chol-pc-dsDNA-iBBr was prepared by annealing Chol-pc-DNA and DNA′-iBBr using sequential incubation at 37° C., 0° C. and RT for 15 min, 10 min and 30 min respectively. 100 μL of Exosomes or BMP2-Exosomes (0.4 μg/μL exosome concentration) were then gently vortexed with 100 μL of preannealed Chol-pc-dsDNA-iBBr tether (2 μM dsDNA tether concentration), followed by three washes with Amicon Ultra Centrifugal Filters (100 k MWCO). The filters were reverse spun to prepare 100 μL of Exo-pc-dsDNA-iBBr (0.4 μg/μL exosome stock concentration, 1 μM initiator concentration).

### Gel Synthesis by Atom Transfer Radical Polymerization:

Plain Gel: 400 μL of PEGMA₃₀₀ monomer, 87.5 μL of PEO₂₀₀₀-iBBr (2 mM stock concentration), 23.6 μL of PEGDMA₇₅₀, (14.8 mM stock concentration), 14.6 μL of CuBr₂/TPMA (12 mM stock concentration; CuBr₂/TPMA=1:6), 20 μL of Glucose Oxidase (100 μM stock concentration), 50 μL of Sodium Pyruvate (2M stock concentration), 100 μL of 10×PBS buffer were thoroughly mixed with 204 μL of H₂O in 4 mL glass vial. Finally, 100 μL of glucose (1 M stock concentration) was added to the vial, followed by irradiation under blue light (450 nm, 3 mW/cm²) for 70 mins at room temperature.

Exosome-tethered Gel: 400 μL of PEGMA₃₀₀ monomer, 87.5 μL of PEO₂₀₀₀-iBBr (2 mM stock concentration), 23.6 μL of PEGDMA₇₅₀, (14.8 mM stock concentration), 14.6 μL of CuBr₂/TPMA (12 mM stock concentration; CuBr₂/TPMA=1:6), 20 μL of Glucose Oxidase (100 μM stock concentration), 504 of Sodium Pyruvate (2M stock concentration), 1004 of 10×PBS buffer were thoroughly mixed with 104 μL of H₂O in 4 mL glass vial. 100 μL of Exo-dsDNA-iBBr (0.4 μg/μL exosome stock concentration, 1 μM initiator concentration) was added and mixed thoroughly. Finally, 1004 of glucose (1 M stock concentration) was added to the vial, followed by irradiation under blue light (450 nm, 3 mW/cm²) for 70 mins at room temperature.

BMP2-Exosome-tethered Gel: BMP2-Exo-dsDNA-iBBr (40 μg exosome, 4 μg BMP2, 1 μM initiator concentration) were used instead of non-labeled exosomes. Gels were prepared using method as described above.

Photocleavable Exosome-tethered Gel: 400 μL of PEGMA₃₀₀ monomer, 87.5 μL of PEO₂₀₀₀-iBBr (2 mM stock concentration), 23.6 μL of PEGDMA₇₅₀, (14.8 mM stock concentration), 14.6 μL of CuBr₂/TPMA (12 mM stock concentration; CuBr₂/TPMA=1:6), 204 of Glucose Oxidase (100 μM stock concentration), 50 μL of Sodium Pyruvate (2M stock concentration), 100 μL of 10×PBS buffer were thoroughly mixed with 104 μL of H₂O in 4 mL glass vial. 100 μL of Exo-pc-dsDNA-iBBr (0.4 μg/μL exosome stock concentration, 1 μM initiator concentration) was added and mixed thoroughly. Finally, 100 μL of glucose (1 M stock concentration) was added to the vial, followed by irradiation under blue light (450 nm, 3 mW/cm²) for 70 mins at room temperature.

Photocleavable BMP2-Exosome-tethered Gel: BMP2-Exo-pc-dsDNA-iBBr (40 μg exosome, 4 μg BMP2, 1 μM initiator concentration) were used instead of non-labeled exosomes. Gels were prepared using method as described above.

Exosome-trapped Gel: 400 μL of PEGMA₃₀₀ monomer, 87.5 μL of PEO₂₀₀₀-iBBr (2 mM stock concentration), 23.6 μL of PEGDMA₇₅₀, (14.8 mM stock concentration), 14.6 μL of CuBr₂/TPMA (12 mM stock concentration; CuBr₂/TPMA=1:6), 20 μL of Glucose Oxidase (100 μM stock concentration), 504 of Sodium Pyruvate (2M stock concentration), 1004 of 10×PBS buffer were thoroughly mixed with 1044 of H₂O in 4 mL glass vial. 100 μL of native exosomes (0.4 μg/μL exosome stock concentration) was added and mixed thoroughly. 100 μL of glucose (1 M stock concentration) was added to the vial, followed by irradiation under blue light (450 nm, 3 mW/cm²) for 70 mins at room temperature.

BMP2-Exosome-trapped Gel: BMP2-Exo (40 μg exosome, 4 μg BMP2) were used instead of non-labeled exosomes. Gels were prepared using method as described above.

BMP2-trapped gels: 400 μL of PEGMA₃₀₀ monomer, 87.5 μL of PEO₂₀₀₀-iBBr (2 mM stock concentration), 23.6 μL of PEGDMA₇₅₀, (14.8 mM stock concentration), 14.6 μL of CuBr₂/TPMA (12 mM stock concentration; CuBr₂/TPMA=1:6), 20 μL of Glucose Oxidase (100 μM stock concentration), 504 of Sodium Pyruvate (2M stock concentration), 1004 of 10×PBS buffer were thoroughly mixed with 1044 of H₂O in 4 mL glass vial. 1004 of BMP2 (0.04 μg/μL BMP2 stock concentration) was added and mixed thoroughly. 100 μL of glucose (1 M stock concentration) was added to the vial, followed by irradiation under blue light (450 nm, 3 mW/cm²) for 70 mins at room temperature.

### Gel Characterization:

Polymerization Kinetics: A plain gel was prepared using the procedure as described above. Samples were collected after different time points of 10, 20, 30, and 70 minutes and were analyzed by NMR for conversion.

Con focal Microscopy: Gels were prepared using dye-labeled Exosomes. Exosome gels were incubated for 24 hours in PBS following which imaging was performed. Imaging was performed using a Carl Zeiss LSM 880 confocal microscope with fixed settings across all of the experimental time points and the images were analyzed using ZEN Black software (Carl Zeiss Microscopy, Thornwood, N.Y.).

Release Kinetics: Gels were prepared using radio-labeled Exosomes. BMP2 was iodinated via chloramine T method (Campbell et al. “Insulin-Like growth factor binding protein (IGFBP) inhibits igf action on human osteosarcoma cells”, Journal of Cellular Physiology, 1991, 149(2):293-300). BMP2 (10 μg) was reacted with 500 μCi ¹²⁵I—Na at 25° C. with stepwise addition of 3 aliquots of dilute chloramine T solution (100 μg/ml). Resulting ¹²⁵I-BMP2 was >97% trichloroacetic acid perceptible with minimal protein aggregate formation. Specific activity of ¹²⁵I-BMP2 was from 55-80 μCi/μg. Exosomes were loaded with ¹²⁵I-BMP2 as described above. To label exosomes, a modified Chloramine T method was employed (manuscript under preparation). Gels were prepared as described above incorporating 10 μg of exosomes with or without ¹²⁵I-BMP2. Release kinetics was assessed in simulated body fluid (SBF; 10% FBS, 0.02% sodium azide, 25 mM HEPES in DMEM) as described previously (manuscript under review). Briefly, gels were put in 12×75 mm polypropylene tubes containing a total volume of 1 ml of SBF. Tubes were incubated at 37° C. and at indicated time-points, SFB was replaced and the retained ¹²⁵I-BMP2/¹²⁵I-exosomes were detected using a Wizard2 2-Detector Gamma Counter (PerkinElmer, Waltham, Mass.). On 25^{th} day, gels with photocleavable tethers were irradiated using 365 nm LEDs (100 mW/cm²) for 2 mins.

### Osteogenic Differentiation:

Alkaline Phosphatase Assay: Circular Exo-gels containing a total of 40 ug exosomes were cut into four quadrants and rinsed thoroughly (four times) with 0.02% EDTA in PBS, the first wash being immediate followed by three more rinses (4-6 hours each rinse). To treat the cells with the gel, cells were plated in a 12-well plate and allowed to adhere overnight. C2C12 cells were incubated with indicated treatments, washed with PBS to remove culture medium, and fixed for 20 min with 10% neutral buffered formalin (Millipore-Sigma, St. Louis, Mo.). Alkaline phosphatase activity was detected using a leukocyte alkaline phosphatase assay kit according to the manufacturer's instructions (Millipore-Sigma, St. Louis, Mo.). Where required, ALP-stained images were converted to CMYK format since this color format is representative of reflected light colors as opposed to emitted light colors (RGB). Since the combination of cyan and magenta form the color blue, these channels were added together and inverted. The average pixel intensity was determined using the image histogram tool in Adobe® Photoshop 7.0 (Adobe® Systems, San Jose, Calif.).

Mineralization: Exo-gels were cut similar to the ones used for ALP assay. MC3T3-E1 (subclone 4) cells were seeded in growth media (ascorbic acid-free α-MEM, 10% FBS, 1% PS). For the treatment with gels, the gels were placed on the plated cells and the growth media supplemented with was 50 μg/ml ascorbic acid and 10 mM β-glycerophosphate changed every 72 hours. As a positive control 100 ng/ml of BMP2 was added along with every change in media. On day 21, cells were fixed in 10% neutral buffered formalin, washed with distilled water three times, and alizarin red stain (Millipore-Sigma, St. Louis, Mo.) was added to the wells and incubated for 1 hr at room temperature. After imaging the cells, quantification of mineralization was performed using an osteogenesis quantitation kit (Millipore-Sigma, St. Louis, Mo.) according to manufacturer's instructions. Briefly, alizarin red stained cells were treated with 10% acetic acid solution for 30 min with shaking, cells were scraped, centrifuged and the dissolved alizarin stain was quantified by measuring the OD at 405 nm (TECAN plate reader, Männedorf, Switzerland) using alizarin red reference standards.

### Results

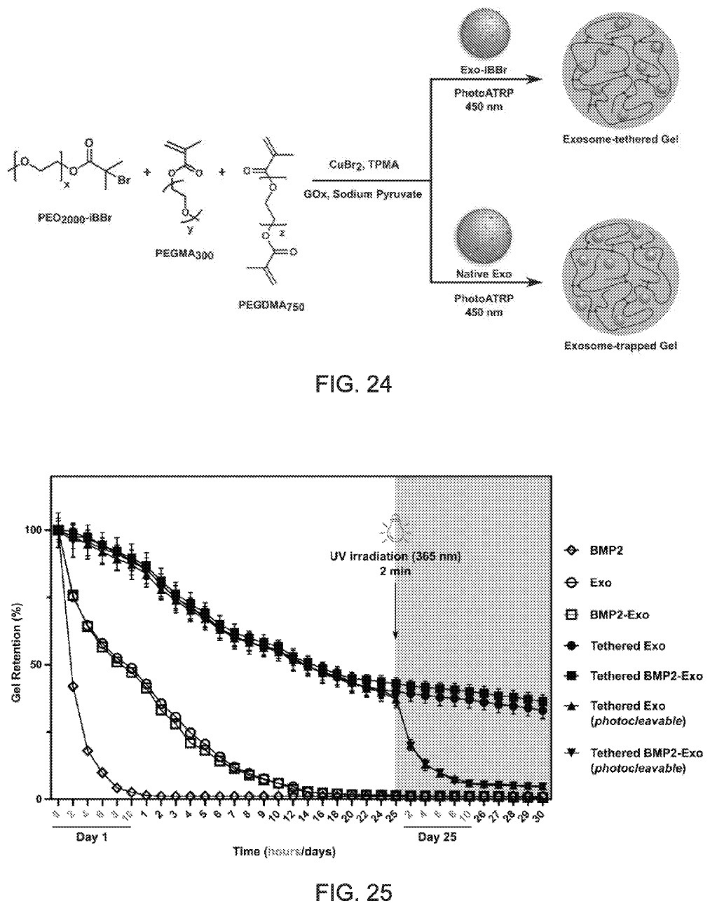

Preparation of exosome-tethered Gels: In order to tether exosomes in the gel network, we prepared exosome macroinitiators (Exo-iBBr) by tethering cholesterol modified DNA macroinitiators (Chol-dsDNA-iBBr) in the exosome membrane as previously reported. Using poly(ethylene glycol) methacrylate (PEGMA, Mₙ=300) as monomer and poly(ethylene glycol) dimethacrylate (PEDGMA, Mₙ=750) as crosslinker, we prepared exosome gels using blue light mediated 02-tolerant ATRP. In addition to the PEO-based initiator, exosome macroinitiator was also used as an additional 5% initiator to tethers the exosomes in the gel network (FIG. 24). Alternatively, exosome can be trapped in the gel network by simply adding them during the gel synthesis (FIG. 24).

In order to confirm the presence of exosomes in the gels, we prepared gels with dye-labeled Exosomes. Exosomes labeled with lipophilic dye (PKH26) were anchored with Cy5-labeled Chol-dsDNA-iBBr. These dual-dye labeled exosomes were used to prepare the gels. Plain (control) gels were prepared using just the Cy5-labeled Chol-dsDNA-iBBr.

Release Kinetics Studies: The release of free BMP2 growth factor and BMP2-labeled exosomes from gels was monitored over a period of 30 days (FIG. 25). To enable detection, exosomes and BMP2 were radiolabeled using I¹²⁵. Gels were prepared with either trapped exosomes or tethered exosomes using exosome macroinitiator. Additionally, we prepared exosome-tethered gels with a photocleavable Cholesterol-modified DNA macroinitiator as previously reported. Table 3 shows the different components that were used to prepare the gels for release kinetics studies.

TABLE 3
Table showing the components of gels
prepared for release kinetics.

| S. No | Gel components | Comments |
|---|---|---|
| 1. | Native Exosomes* | Trapped exosomes |
| 2. | Exo*-iBBr | Tethered exosomes |
| 3. | Exo*-pc-iBBr | Tethered exosomes with photo-cleavable |
| | | group |
| 4. | Exo(BMP2)* | Trapped bmp2-labeled exosomes |
| 5. | Exo(BMP2)*-iBBr | Tethered-Bmp2-labeled exosomes |
| 6. | Exo(BMP2)*-pc- | Tethered-Bmp2-labeled exosomes with |
| | iBBr | photo-cleavable group |
| 7. | BMP2* | Trapped Bmp2 |

*Labeled with radioactive |¹²⁵

The trapped BMP2 completely cleared out of gel network in 24 hours, while the trapped exosomes and BMP2-labeled exosomes after 12 days (FIG. 25). Interestingly, around 30% of tethered exosomes and BMP2-labeled exosomes were observed to be retained in the gel after 30 days. In the case of gels with photocleavable groups, we irradiated the gels for 2 mins using UV light on day 30 and observed a burst release of exosomes within the following 24 hours.

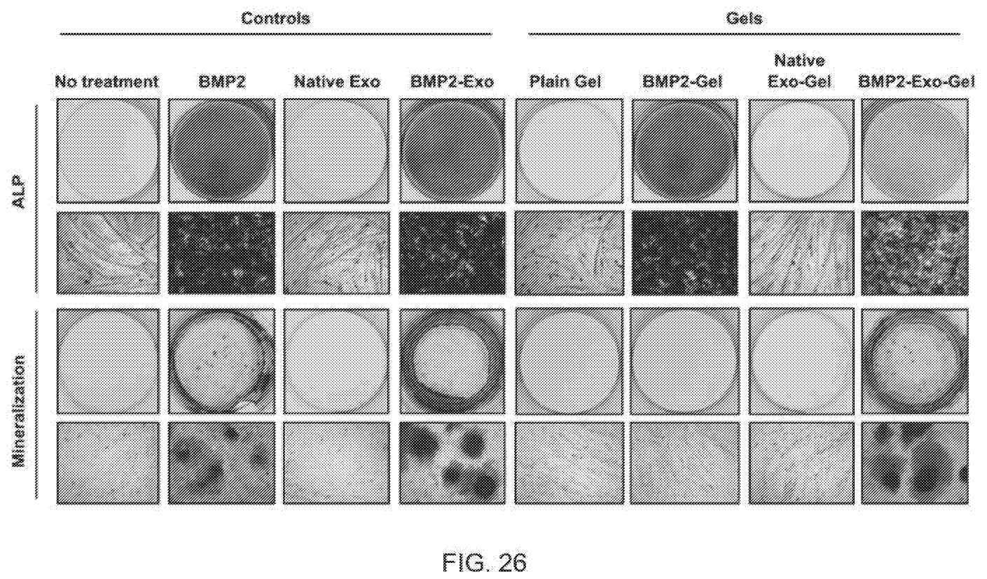

Osteogenesis Differentiation Studies: In order to evaluate the biological activity of BMP2 loaded gels, we used BMP2-loaded exosomes to prepare the gels. We assessed two bone formation markers—alkaline phosphatase (ALP) and mineral deposits. ALP is an early bone differentiation marker and mineral deposits are late bone differentiation marker. ALP assay was concluded in 72 hours without any media change. The cells treated with BMP2, BMP2-Exosomes, both in liquid (control) and solid-phase (gel) resulted in osteogenic differentiation of C2C12 cells as evidenced by upregulation in ALP expression (FIG. 26).

The mineralization assay was performed over a period of 28 days with media change every 72 hours using MC3T3 cells. 100 nanograms per milliliter (ng/ml) BMP2 was supplemented during every media change for experiments involving liquid phase assays. On the contrary, cells incubated with gels did not receive any BMP2 with the fresh media. Cells receiving only liquid-phase BMP2 and solid phase BMP2-Exosomes resulted in mineral deposits suggesting when tethered to gels, BMP2-EVs are slowly released into the media that effect cell differentiation (FIG. 26). While, trapped BMP2 and BMP2-exosomes did not result formation of mineral deposits.

Exosome, 30-150 nm vesicles, secreted by typically every cell in the body are of growing importance. However, rapid clearance of exosomes from the blood pos-injections limits their therapeutic potential. Alternatively, hydrogels-based delivery systems can be used for localized delivery of exosomes for therapeutic applications. Here, a well-defined exosome-tethered PEO-based hydrogel for controlled and sustained release of therapeutic exosomes is reported. Using cholesterol-modified DNA tethers, outer membrane of exosomes was functionalized with initiator to graft polymers in the presence of crosslinkers using atom transfer radical polymerization. It was observed that the strategy of exosome tethering in the gel network allows a sustained release of exosomes over a period of one month as compared to exosomes directly trapped in the gels. Further, use of photocleavable linker between the exosomes and the gel network allowed temporal control over the release profile of exosomes. Further, it was confirmed the therapeutic potential of the gels through the delivery of BMP2 growth factor for osteogenic differentiation.

While several examples and embodiments are shown in the accompanying figures and described hereinabove in detail, other examples and embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A tethered extracellular vesicle comprising:
an extracellular vesicle, such as a microvesicle or an exosome, obtained from a living cell, tissue, organ, or organism;
a hydrophobically-modified first oligonucleotide anchored to the extracellular vesicle; and
a second oligonucleotide hybridized to the first oligonucleotide linked to a member of a binding pair, a therapeutic agent, a surface, or a polymer.

2. The tethered extracellular vesicle of claim 1, wherein the second oligonucleotide is linked to a polymer, such as a polyacrylate, a polymethacrylate, a polyacrylamide, a polypeptide, a polymethacrylamide, a polypeptide, a polystyrene, a polyethylene oxide, a poly(organo)phosphazene, a poly-L-lysine, a polyethyleneimine, a poly-d,l-lactide-co-glycolide, or a poly(alkylcyanoacrylate).

3. The tethered extracellular vesicle of claim 2, wherein the polymer has a saturated carbon backbone, is prepared from one or more ethylenically unsaturated monomers, and/or has a PDI of less than 2.0.

4. The tethered extracellular vesicle of claim 2, wherein the polymer is an acrylic polymer and wherein the acrylic polymer optionally comprises pendant poly(ethylene oxide) groups comprising the structure —(O—CH₂—CH₂—)ₙ, where n is 100 or less, 20 or less or 10 or less, or has an Mₙ of 200 or less; zwitterionic groups; or methylsulfinyl terminated alkyl groups.

5. The tethered extracellular vesicle of claim 1, wherein the second oligonucleotide is linked to a biologically active agent, such as a therapeutic agent, or a binding reagent, such as an antibody, an antibody fragment, or an aptamer.

6. The tethered extracellular vesicle of claim 5, wherein the second oligonucleotide is linked to a binding reagent complexed with a biologically active agent, such as a therapeutic agent.

7. The tethered extracellular vesicle of claim 1, wherein the extracellular vesicle comprises a therapeutic agent, such as a therapeutic loaded inside the lumen of the extracellular vesicle or on the membrane surface.

8. A composition comprising the tethered extracellular vesicle of claim 1, and a pharmaceutically-acceptable excipient.

9. A hydrogel comprising two or more of the tethered extracellular vesicles of claim 2, wherein the polymer of the two or more tethered extracellular vesicles is cross-linked with a cross-linker, wherein the polymer optionally comprises a saturated carbon backbone and/or is functionalized with poly(ethylene oxide)-containing groups, and the cross-linker comprises poly(ethylene oxide)

10. The hydrogel of claim 9, comprising a biologically active agent, such as a therapeutic agent, such as a therapeutic loaded inside the lumen of the extracellular vesicle or on the membrane surface and/or a biologically active agent, such as a therapeutic agent, tethered to the extracellular vesicle by attachment to, or complexing with the hydrophobically-modified oligonucleotide.

11. A tethered extracellular vesicle comprising:
an extracellular vesicle, such as a microvesicle or an exosome, obtained from a living cell, tissue, organ, or organism; and
a hydrophobically-modified oligonucleotide anchored to the extracellular vesicle and linked to a polymer.

12. The tethered extracellular vesicle of claim 11, wherein the polymer is a polyacrylate, a polymethacrylate, a polyacrylamide, a polymethacrylamide, a polypeptide, a polystyrene, a polyethylene oxide, a poly(organo)phosphazene, a poly-L-lysine, a polyethyleneimine, a poly-d,l-lactide-co-glycolide, or a poly(alkylcyanoacrylate).

13. The tethered extracellular vesicle of claim 11, wherein the polymer has a saturated carbon backbone, is prepared from one or more ethylenically unsaturated monomers, and/or PDI of less than 2.0.

14. The tethered extracellular vesicle of claim 11, wherein the polymer is an acrylic polymer and wherein the acrylic polymer optionally comprises pendant poly(ethylene oxide) groups having the structure —(O—CH₂—CH₂—)ₙ, where n is 100 or less, 20 or less or 10 or less, or has an Mₙ of 200 or less; zwitterionic groups; or methylsulfinyl terminated alkyl groups.

15. A composition comprising the tethered extracellular vesicle of claim 11, and a pharmaceutically-acceptable excipient.

16. A hydrogel comprising two or more of the tethered extracellular vesicles of claim 11, wherein the polymer of the two or more tethered extracellular vesicles is cross-linked with a cross-linker, wherein the polymer optionally comprises a saturated carbon backbone and/or is functionalized with poly(ethylene oxide)-containing groups, and the cross-linker comprises poly(ethylene oxide).

17. The hydrogel of claim 16, comprising a biologically active agent, such as a therapeutic agent, such as a therapeutic loaded inside the lumen of the extracellular vesicle or on the membrane surface and/or a biologically active agent, such as a therapeutic agent, tethered to the extracellular vesicle by attachment to, or complexing with the hydrophobically-modified oligonucleotide.

18. A method of making a tethered extracellular vesicle, comprising:
anchoring a hydrophobically-modified oligonucleotide to an extracellular vesicle, such as a microvesicle or an exosome, obtained from a living cell, tissue, organ, or organism;
hybridizing to the hydrophobically-modified oligonucleotide a second oligonucleotide complementary to the hydrophobically-modified oligonucleotide and linked to a member of a binding pair, a therapeutic agent, a surface, a polymer initiator group, or a polymer; or
anchoring a hydrophobically-modified oligonucleotide comprising a polymer initiator group to the extracellular vesicle, such as a microvesicle or an exosome, obtained from a living cell, tissue, organ, or organism; and
polymerizing a polymer in a polymerization reaction from the polymer initiator group.

19. (canceled)

20. The method of claim 18, wherein the polymer is prepared from one or more ethylenically unsaturated monomers and/or has a PDI of less than 2.0.

21. (canceled)

22. (canceled)

23. (canceled)

24. The method of claim 18, wherein the polymerization reaction is conducted using controlled radical polymerization, such as by atom transfer radical polymerization (ATRP), such as Activators ReGenerated by Electron Transfer (ARGET) ATRP, Initiators for Continuous Activator Regeneration (ICAR) ATRP, supplemental activator and reducing agent atom transfer radical polymerization (SARA) ATRP, electrochemically-controlled ATRP (e-ATRP), or photoinduced ATRP.

25-35. (canceled)


SEQUENCE LISTING (16 sequences)

SEQ ID NO: 1 - protein, 5 aa, Artificial Sequence
  cell adhesion peptide
IKVAV

SEQ ID NO: 2 - protein, 4 aa, Artificial Sequence
  cell adhesion peptide
RGDS

SEQ ID NO: 3 - protein, 6 aa, Artificial Sequence
  cell adhesion peptide
KQAGDV

SEQ ID NO: 4 - protein, 6 aa, Artificial Sequence
  cell adhesion peptide
VAPGVG

SEQ ID NO: 5 - protein, 6 aa, Artificial Sequence
  cell adhesion peptide
APGVGV

SEQ ID NO: 6 - protein, 6 aa, Artificial Sequence
  cell adhesion peptide
PGVGVA

SEQ ID NO: 7 - protein, 5 aa, Artificial Sequence
  cell adhesion peptide
GVGVA

SEQ ID NO: 8 - protein, 4 aa, Artificial Sequence
  cell adhesion peptide
VAPG

SEQ ID NO: 9 - protein, 6 aa, Artificial Sequence
  cell adhesion peptide
VGVAPG

SEQ ID NO: 10 - protein, 4 aa, Artificial Sequence
  cell adhesion peptide
VGVA

SEQ ID NO: 11 - protein, 5 aa, Artificial Sequence
  cell adhesion peptide
VAPGV

SEQ ID NO: 12 - protein, 6 aa, Artificial Sequence
  cell adhesion peptide
GVAPGV

SEQ ID NO: 13 - DNA, 50 bp, Artificial Sequence
  AS1411 with complementary 18-mer DNA tether
ggtggtggtggttgtggtggtggtggttagctatgggatccaactgcagt

SEQ ID NO: 14 - DNA, 20 bp, Artificial Sequence
  aminated DNA 18-mer tether (NH2-C12-alphaFC)
ttatgggatccaactgcagt

SEQ ID NO: 15 - DNA, 18 bp, Artificial Sequence
  18-mer DNA tether
actgcagttggatcccat

SEQ ID NO: 16 - DNA, 18 bp, Artificial Sequence
  complementary 18-mer DNA tether
atgggatccaactgcagt